SEQUENCE LISTING (135 sequences)

SEQ ID NO: 1 - protein, 736 aa, synthetic construct
  REGION 1..736: note = Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 2211 bp, synthetic construct
  misc_feature 1..2211: note = Synthetic
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3 - protein, 743 aa, synthetic construct
  REGION 1..743: note = Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - DNA, 2229 bp, synthetic construct
  misc_feature 1..2229: note = Synthetic
  source 1..2229: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctctagaactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggtaccggcagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggttgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 5 - protein, 735 aa, synthetic construct
  REGION 1..735: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 6 - DNA, 2210 bp, synthetic construct
  misc_feature 1..2210: note = Synthetic
  source 1..2210: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcacggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 7 - protein, 735 aa, synthetic construct [sequence omitted]

SEQ ID NO: 8 - DNA, 2207 bp, synthetic construct
  misc_feature 1..2207: note = Synthetic
  source 1..2207: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 9 - protein, 735 aa, synthetic construct
  REGION 1..735: note = Synthetic
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 10 - DNA, 2211 bp, synthetic construct
  misc_feature 1..2211: note = Synthetic
  source 1..2211: mol_type = other DNA
atggctgctgacggttatcttccagattggctcgaggacaacctttctgaaggcattcgtgagtggtgggctctgaaacctggagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 11 - protein, 734 aa, synthetic construct
  REGION 1..734: note = Synthetic
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 12 - DNA, 2202 bp, synthetic construct
  misc_feature 1..2202: note = Synthetic
  source 1..2202: mol_type = other DNA
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctg

SEQ ID NO: 13 - protein, 724 aa, synthetic construct
  REGION 1..724: note = Synthetic
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - DNA, 2175 bp, synthetic construct
  misc_feature 1..2175: note = Synthetic
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 15 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - DNA, 2210 bp, synthetic construct
  misc_feature 1..2210: note = Synthetic
  source 1..2210: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacacctcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 17 - protein, 737 aa, synthetic construct
  REGION 1..737: note = Synthetic
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 18 - DNA, 2213 bp, synthetic construct
  misc_feature 1..2213: note = Synthetic
  source 1..2213: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaaatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 19 - protein, 738 aa, synthetic construct
  REGION 1..738: note = Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 20 - DNA, 2216 bp, synthetic construct
  misc_feature 1..2216: note = Synthetic
  source 1..2216: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataacacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 21 - protein, 737 aa, synthetic construct
  REGION 1..737: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 22 - DNA, 2217 bp, synthetic construct
  misc_feature 1..2217: note = Synthetic
  source 1..2217: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 23 - protein, 736 aa, synthetic construct
  REGION 1..736: note = Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 24 - DNA, 3401 bp, synthetic construct
  misc_feature 1..3401: note = Synthetic
  source 1..3401: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaa

SEQ ID NO: 25 - protein, 15 aa, synthetic construct
  REGION 1..15: note = Synthetic
GGGGGGGGGGGGGGG

SEQ ID NO: 26 - protein, 15 aa, synthetic construct
  REGION 1..15: note = Synthetic
GGGGSGGGGSGGGGS

SEQ ID NO: 27 - DNA, 81 bp, synthetic construct
  misc_feature 1..81: note = Synthetic
  misc_feature 42..62: note = n is a, c, g, or t
  source 1..81: mol_type = other DNA
gtattccttggttttgaacccaaccggtctgcgcctgtgctnnnnnnnnnnnnnnnnnnnnntgggcactctggtggtttg

SEQ ID NO: 28 - DNA, 30 bp, synthetic construct
  misc_feature 1..30: note = Synthetic
  source 1..30: mol_type = other DNA
cactcatcgaccaatacttgtactatctct

SEQ ID NO: 29 - DNA, 26 bp, synthetic construct
  misc_feature 1..26: note = Synthetic
  source 1..26: mol_type = other DNA
gtattccttggttttgaacccaaccg

SEQ ID NO: 30 - DNA, 23 bp, synthetic construct
  misc_feature 1..23: note = Synthetic
  source 1..23: mol_type = other DNA
tgttcccatagtaacgccaatag

SEQ ID NO: 31 - DNA, 26 bp, synthetic construct
  misc_feature 1..26: note = Synthetic
  source 1..26: mol_type = other DNA
gtacttggcatatgatacacttgatg

SEQ ID NO: 32 - DNA, 21 bp, synthetic construct
  misc_feature 1..21: note = Synthetic
  source 1..21: mol_type = other DNA
ggaacccctagtgatggagtt

SEQ ID NO: 33 - DNA, 16 bp, synthetic construct
  misc_feature 1..16: note = Synthetic
  source 1..16: mol_type = other DNA
cggcctcagtgagcga

SEQ ID NO: 34 - DNA, 17 bp, synthetic construct
  misc_feature 1..17: note = Synthetic
  source 1..17: mol_type = other DNA
cggacccagcaacatgg

SEQ ID NO: 35 - DNA, 17 bp, synthetic construct
  misc_feature 1..17: note = Synthetic
  source 1..17: mol_type = other DNA
cggacccagcaacatgg

SEQ ID NO: 36 - DNA, 21 bp, synthetic construct
  misc_feature 1..21: note = Synthetic
  source 1..21: mol_type = other DNA
tggttgagacacgttgttgtc

SEQ ID NO: 37 - DNA, 21 bp, synthetic construct
  misc_feature 1..21: note = Synthetic
  source 1..21: mol_type = other DNA
tggttgagacacgttgttgtc

SEQ ID NO: 38 - DNA, 24 bp, synthetic construct
  misc_feature 1..24: note = Synthetic
  source 1..24: mol_type = other DNA
ttacggtaaactgcccacttggca

SEQ ID NO: 39 - DNA, 21 bp, synthetic construct
  misc_feature 1..21: note = Synthetic
  source 1..21: mol_type = other DNA
cactccctctctgcgcgctcg

SEQ ID NO: 40 - DNA, 27 bp, synthetic construct
  misc_feature 1..27: note = Synthetic
  source 1..27: mol_type = other DNA
agggaagaaactacatacctggaccca

SEQ ID NO: 41 - DNA, 24 bp, synthetic construct
  misc_feature 1..24: note = Synthetic
  source 1..24: mol_type = other DNA
aagaaactggctacctggaccctg

SEQ ID NO: 42 - DNA, 52 bp, synthetic construct
  misc_feature 1..52: note = Synthetic
  source 1..52: mol_type = other DNA
acgacgctcttccgatctgacagaatcaacaaacgctaaaattcagtgtggc

SEQ ID NO: 43 - DNA, 53 bp, synthetic construct
  misc_feature 1..53: note = Synthetic
  misc_feature 19: note = n is a, c, g, or t
  source 1..53: mol_type = other DNA
acgacgctcttccgatctngacagaatcaacaaacgctaaaattcagtgtggc

SEQ ID NO: 44 - DNA, 54 bp, synthetic construct
  misc_feature 1..54: note = Synthetic
  misc_feature 19..20: note = n is a, c, g, or t
  source 1..54: mol_type = other DNA
acgacgctcttccgatctnngacagaatcaacaaacgctaaaattcagtgtggc

SEQ ID NO: 45 - DNA, 55 bp, synthetic construct
  misc_feature 1..55: note = Synthetic
  misc_feature 19..21: note = n is a, c, g, or t
  source 1..55: mol_type = other DNA
acgacgctcttccgatctnnngacagaatcaacaaacgctaaaattcagtgtggc

SEQ ID NO: 46 - DNA, 56 bp, synthetic construct
  misc_feature 1..56: note = Synthetic
  misc_feature 19..22: note = n is a, c, g, or t
  source 1..56: mol_type = other DNA
acgacgctcttccgatctnnnngacagaatcaacaaacgctaaaattcagtgtggc

SEQ ID NO: 47 - DNA, 57 bp, synthetic construct
  misc_feature 1..57: note = Synthetic
  misc_feature 19..23: note = n is a, c, g, or t
  source 1..57: mol_type = other DNA
acgacgctcttccgatctnnnnngacagaatcaacaaacgctaaaattcagtgtggc

SEQ ID NO: 48 - DNA, 58 bp, synthetic construct
  misc_feature 1..58: note = Synthetic
  misc_feature 19..24: note = n is a, c, g, or t
  source 1..58: mol_type = other DNA
acgacgctcttccgatctnnnnnngacagaatcaacaaacgctaaaattcagtgtggc

SEQ ID NO: 49 - DNA, 59 bp, synthetic construct
  misc_feature 1..59: note = Synthetic
  misc_feature 19..25: note = n is a, c, g, or t
  source 1..59: mol_type = other DNA
acgacgctcttccgatctnnnnnnngacagaatcaacaaacgctaaaattcagtgtggc

SEQ ID NO: 50 - DNA, 53 bp, synthetic construct
  misc_feature 1..53: note = Synthetic
  source 1..53: mol_type = other DNA
ggagttcagacgtgtgctcttccgatctcatctctgtcctgccaaaccatacc

SEQ ID NO: 51 - DNA, 54 bp, synthetic construct
  misc_feature 1..54: note = Synthetic
  misc_feature 29: note = n is a, c, g, or t
  source 1..54: mol_type = other DNA
ggagttcagacgtgtgctcttccgatctncatctctgtcctgccaaaccatacc

SEQ ID NO: 52 - DNA, 55 bp, synthetic construct
  misc_feature 1..55: note = Synthetic
  misc_feature 29..30: note = n is a, c, g, or t
  source 1..55: mol_type = other DNA
ggagttcagacgtgtgctcttccgatctnncatctctgtcctgccaaaccatacc

SEQ ID NO: 53 - DNA, 56 bp, synthetic construct
  misc_feature 1..56: note = Synthetic
  misc_feature 29..31: note = n is a, c, g, or t
  source 1..56: mol_type = other DNA
ggagttcagacgtgtgctcttccgatctnnncatctctgtcctgccaaaccatacc

SEQ ID NO: 54 - DNA, 57 bp, synthetic construct
  misc_feature 1..57: note = Synthetic
  misc_feature 29..32: note = n is a, c, g, or t
  source 1..57: mol_type = other DNA
ggagttcagacgtgtgctcttccgatctnnnncatctctgtcctgccaaaccatacc

SEQ ID NO: 55 - DNA, 58 bp, synthetic construct
  misc_feature 1..58: note = Synthetic
  misc_feature 29..33: note = n is a, c, g, or t
  source 1..58: mol_type = other DNA
ggagttcagacgtgtgctcttccgatctnnnnncatctctgtcctgccaaaccatacc

SEQ ID NO: 56 - DNA, 59 bp, synthetic construct
  misc_feature 1..59: note = Synthetic
  misc_feature 29..34: note = n is a, c, g, or t
  source 1..59: mol_type = other DNA
ggagttcagacgtgtgctcttccgatctnnnnnncatctctgtcctgccaaaccatacc

SEQ ID NO: 57 - DNA, 60 bp, synthetic construct
  misc_feature 1..60: note = Synthetic
  misc_feature 29..35: note = n is a, c, g, or t
  source 1..60: mol_type = other DNA
ggagttcagacgtgtgctcttccgatctnnnnnnncatctctgtcctgccaaaccatacc

SEQ ID NO: 58 - protein, 492 aa, synthetic construct
  REGION 1..492: note = Synthetic
GACAGAATCAACAAACGCTAAAATTCAGTGTGGCCGGACCCAGCAACATGGCTGTCCAGGGAAGAAACNNNNNNCCTGGACCCNNNTACCGACAACAACGTGTCTCAACCACTGTGACTCAAAACAACAACAGCGAATTTGCTTGGCCTGGAGCTTCTTCTTGGGCTCTCAATGGACGTAATAGCTTGATGAATCCTGGACCTGCTATGGCCAGCCACAAAGAAGGAGAGGACCGTTTCTTTCCTTTGTCTGGATCTTTAATTTTTGGCAAACAAGGAACTGGAAGAGACAACGTGGATGCGGACAAAGTCATGATAACCAACGAAGAAGAAATTAAAACTACTAACCCGGTAGCAACGGAGTCCTATGGACAAGTGGCCACAAACCACCAGAGTGCCCAANNNNNNNNNNNNNNNNNNNNNGCACAGGCGCAGACCGGTTGGGTTCAAAACCAAGGAATACTTCCGGGTATGGTTTGGCAGGACAGAGATG

SEQ ID NO: 59 - protein, 83 aa, synthetic construct
  REGION 1..83: note = Synthetic
MAADGYLPDWLEDNLSEGIREWWDLKPGAPKPKANQQKQDDGRGLVLPGYKYLGPFNGLDKGEPVNAADAAALEHDKAYDQQL

SEQ ID NO: 60 - protein, 83 aa, synthetic construct
  REGION 1..83: note = Synthetic
MAADGYLPDWLEDNLSEGIREWWALKPGAPQPKANQQHQDNARGLVLPGYKYLGPGNGLDKGEPVNAADAAALEHDKAYDQQL

SEQ ID NO: 61 - protein, 83 aa, synthetic construct
  REGION 1..83: note = Synthetic
MAADGYLPDWLEDNLSEGIREWWALKPGAPKPKANQQKQDDGRGLVLPGYKYLGPFNGLDKGEPVNAADAAALEHDKAYDQQL

SEQ ID NO: 62 - protein, 83 aa, synthetic construct; identical to SEQ ID NO: 59
  REGION 1..83: note = Synthetic

SEQ ID NO: 63 - protein, 83 aa, synthetic construct
  REGION 1..83: note = Synthetic
MAADGYLPDWLEDNLSEGIREWWDLKPGAPKLKANQQKQDDGRGLVLPGYKYLGPFHGLDKGEPVNAADAAALEHDKAYDQQL

SEQ ID NO: 64 - protein, 83 aa, synthetic construct
  REGION 1..83: note = Synthetic
MAADGYLPDWLEDNLSEGIREWWDLKPGAPKPKANQQKQDDGRGLVLPGYEYLGPFNGLDKGEPVNAADAAALEHDKAYDQQL

SEQ ID NO: 65 - protein, 82 aa, synthetic construct
  REGION 1..82: note = Synthetic
MTDGYLPDWLEDNLSEGIREWWALQPGAPKPKANQQHQDNARGLVLPGYKYLGPGNGLDKGEPVNAADAAALEHDKAYDQQL

SEQ ID NO: 66 - protein, 83 aa, synthetic construct
  REGION 1..83: note = Synthetic
MAADGYLPDWLEDNLSEGIREWWDLKPGAPKPKANQQKQDNGRGLVLPGYKYLGPFNGLDKGEPVNAADAAALEHDKAYDQQL

SEQ ID NO: 67 - protein, 83 aa, synthetic construct; identical to SEQ ID NO: 66
  REGION 1..83: note = Synthetic

SEQ ID NO: 68 - protein, 83 aa, synthetic construct; identical to SEQ ID NO: 59
  REGION 1..83: note = Synthetic

SEQ ID NO: 69 - protein, 83 aa, synthetic construct; identical to SEQ ID NO: 59
  REGION 1..83: note = Synthetic

SEQ ID NO: 70 - protein, 83 aa, synthetic construct; identical to SEQ ID NO: 59
  REGION 1..83: note = Synthetic

SEQ ID NO: 71 - protein, 83 aa, synthetic construct; identical to SEQ ID NO: 59
  REGION 1..83: note = Synthetic

SEQ ID NO: 72 - protein, 83 aa, synthetic construct
  REGION 1..83: note = Synthetic
MAADGYLPDWLEGNLSEGIREWWDLKPGAPKPKANQQKQDDGRGLVLPGYRYLGPFNGLDKGEPVNEADAAALEHDKAYDKQL

SEQ ID NO: 73 - protein, 83 aa, synthetic construct
  REGION 1..83: note = Synthetic
MAADGYLPDWLEDNLSEGIREWWDLKPGAPKPKANQQKQDDGRGLVLPGYKYLGPFNGLDKGEPVNEADAAALEHDKAYDKQL

SEQ ID NO: 74 - protein, 83 aa, synthetic construct; identical to SEQ ID NO: 73
  REGION 1..83: note = Synthetic

SEQ ID NO: 75 - protein, 83 aa, synthetic construct; identical to SEQ ID NO: 73
  REGION 1..83: note = Synthetic

SEQ ID NO: 76 - protein, 83 aa, synthetic construct; identical to SEQ ID NO: 73
  REGION 1..83: note = Synthetic

SEQ ID NO: 77 - protein, 83 aa, synthetic construct; identical to SEQ ID NO: 59
  REGION 1..83: note = Synthetic

SEQ ID NO: 78 - protein, 83 aa, synthetic construct; identical to SEQ ID NO: 59
  REGION 1..83: note = Synthetic

SEQ ID NO: 79 - protein, 83 aa, synthetic construct; identical to SEQ ID NO: 59
  REGION 1..83: note = Synthetic

SEQ ID NO: 80 - protein, 83 aa, synthetic construct; identical to SEQ ID NO: 61
  REGION 1..83: note = Synthetic

SEQ ID NO: 81 - protein, 83 aa, synthetic construct; identical to SEQ ID NO: 61
  REGION 1..83: note = Synthetic

SEQ ID NO: 82 - protein, 83 aa, synthetic construct; identical to SEQ ID NO: 59
  REGION 1..83: note = Synthetic

SEQ ID NO: 83 - protein, 83 aa, synthetic construct; identical to SEQ ID NO: 59
  REGION 1..83: note = Synthetic

SEQ ID NO: 84 - protein, 83 aa, synthetic construct; identical to SEQ ID NO: 59
  REGION 1..83: note = Synthetic

SEQ ID NO: 85 - protein, 83 aa, synthetic construct; identical to SEQ ID NO: 59
  REGION 1..83: note = Synthetic

SEQ ID NO: 86 - protein, 83 aa, synthetic construct; identical to SEQ ID NO: 59
  REGION 1..83: note = Synthetic

SEQ ID NO: 87 - protein, 83 aa, synthetic construct; identical to SEQ ID NO: 59
  REGION 1..83: note = Synthetic

SEQ ID NO: 88 - protein, 83 aa, synthetic construct; identical to SEQ ID NO: 59
  REGION 1..83: note = Synthetic

SEQ ID NO: 89 - protein, 83 aa, synthetic construct; identical to SEQ ID NO: 59
  REGION 1..83: note = Synthetic

SEQ ID NO: 90 - protein, 83 aa, synthetic construct; identical to SEQ ID NO: 59
  REGION 1..83: note = Synthetic

SEQ ID NO: 91 - protein, 83 aa, synthetic construct; identical to SEQ ID NO: 59
  REGION 1..83: note = Synthetic

SEQ ID NO: 92 - protein, 83 aa, synthetic construct; identical to SEQ ID NO: 59
  REGION 1..83: note = Synthetic

SEQ ID NO: 93 - protein, 83 aa, synthetic construct
  REGION 1..83: note = Synthetic
MAADGYLPDWLEDNLSEGIREWWALKPGAPQPKANQQKQDDGRGLVLPGYKYLGPFNGLDKGEPVNEADAAALEHDKAYDQQL

SEQ ID NO: 94 - protein, 83 aa, synthetic construct; identical to SEQ ID NO: 93
  REGION 1..83: note = Synthetic

SEQ ID NO: 95 - protein, 83 aa, synthetic construct; identical to SEQ ID NO: 93
  REGION 1..83: note = Synthetic

SEQ ID NO: 96 - protein, 83 aa, synthetic construct; identical to SEQ ID NO: 59
  REGION 1..83: note = Synthetic

SEQ ID NO: 97 - protein, 83 aa, synthetic construct; identical to SEQ ID NO: 59
  REGION 1..83: note = Synthetic

SEQ ID NO: 98 - protein, 83 aa, synthetic construct
  REGION 1..83: note = Synthetic
MVADGYLPDWLEDNLSEGIREWWDLKPGAPKPKANQQKQDDGRGLVLPGYKYLGPFNGLDKGEPVNAADAAALEHDKAYDQQL

SEQ ID NO: 99 - protein, 83 aa, synthetic construct; identical to SEQ ID NO: 59
  REGION 1..83: note = Synthetic

SEQ ID NO: 100 - protein, 83 aa, synthetic construct; identical to SEQ ID NO: 59
  REGION 1..83: note = Synthetic

SEQ ID NO: 101 - protein, 83 aa, synthetic construct; identical to SEQ ID NO: 59
  REGION 1..83: note = Synthetic

SEQ ID NO: 102 - protein, 83 aa, synthetic construct; identical to SEQ ID NO: 59
  REGION 1..83: note = Synthetic

SEQ ID NO: 103 - protein, 83 aa, synthetic construct; identical to SEQ ID NO: 59
  REGION 1..83: note = Synthetic

SEQ ID NO: 104 - protein, 83 aa, synthetic construct; identical to SEQ ID NO: 59
  REGION 1..83: note = Synthetic

SEQ ID NO: 105 - protein, 83 aa, synthetic construct; identical to SEQ ID NO: 61
  REGION 1..83: note = Synthetic

SEQ ID NO: 106 - protein, 83 aa, synthetic construct; identical to SEQ ID NO: 59
  REGION 1..83: note = Synthetic

SEQ ID NO: 107 - protein, 83 aa, synthetic construct; identical to SEQ ID NO: 59
  REGION 1..83: note = Synthetic

SEQ ID NO: 108 - protein, 83 aa, synthetic construct; identical to SEQ ID NO: 59
  REGION 1..83: note = Synthetic

SEQ ID NO: 109 - protein, 83 aa, synthetic construct
  REGION 1..83: note = Synthetic
MAADGYLPDWLEDTLSEGIRQWWKLKPGPPPPKPAERHKDDSRGLVLPGYKYLGPGNGLDKGEPVNAADAAALEHDKAYDQQL

SEQ ID NO: 110 - protein, 83 aa, synthetic construct; identical to SEQ ID NO: 109
  REGION 1..83: note = Synthetic

SEQ ID NO: 111 - protein, 83 aa, synthetic construct
  REGION 1..83: note = Synthetic
MAADGYLPDWLEDTLSEGIRQRWKLKPGPPPPEPAERHKDDSRGLVLPGYKYLGPFNGLDKGEPVNEADAAALEHDKAYDRQL

SEQ ID NO: 112 - protein, 83 aa, synthetic construct; identical to SEQ ID NO: 111
  REGION 1..83: note = Synthetic

SEQ ID NO: 113 - protein, 83 aa, synthetic construct
  REGION 1..83: note = Synthetic
MAADGYLPDWLEDTLSEGIRQWWKLKPGPPPPKPAERHKDDSRGLVLPGYKYLGPFNGLDKGEPVNEADAAALEHDKAYDRQL

SEQ ID NO: 114 - protein, 83 aa, synthetic construct
  REGION 1..83: note = Synthetic
MAADGYLPDWLEDTLSEGIRQWWKLKPGPPPPKPAERHRDDSRGLVLPGYKYLGPFNGLDKGEPVNEADAAALEHDKAYDRQL

SEQ ID NO: 115 - protein, 83 aa, synthetic construct; identical to SEQ ID NO: 114
  REGION 1..83: note = Synthetic

SEQ ID NO: 116 - protein, 83 aa, synthetic construct; identical to SEQ ID NO: 113
  REGION 1..83: note = Synthetic

SEQ ID NO: 117 - protein, 83 aa, synthetic construct; identical to SEQ ID NO: 113
  REGION 1..83: note = Synthetic

SEQ ID NO: 118 - protein, 83 aa, synthetic construct; identical to SEQ ID NO: 113
  REGION 1..83: note = Synthetic

SEQ ID NO: 119 - protein, 83 aa, synthetic construct; identical to SEQ ID NO: 113
  REGION 1..83: note = Synthetic

SEQ ID NO: 120 - protein, 83 aa, synthetic construct; identical to SEQ ID NO: 113
  REGION 1..83: note = Synthetic

SEQ ID NO: 121 - protein, 83 aa, synthetic construct; identical to SEQ ID NO: 113
  REGION 1..83: note = Synthetic

SEQ ID NO: 122 - protein, 83 aa, synthetic construct; identical to SEQ ID NO: 113
  REGION 1..83: note = Synthetic

SEQ ID NO: 123 - protein, 83 aa, synthetic construct; identical to SEQ ID NO: 113
  REGION 1..83: note = Synthetic

SEQ ID NO: 124 - protein, 83 aa, synthetic construct; identical to SEQ ID NO: 113
  REGION 1..83: note = Synthetic

SEQ ID NO: 125 - protein, 83 aa, synthetic construct; identical to SEQ ID NO: 113
  REGION 1..83: note = Synthetic

SEQ ID NO: 126 - protein, 83 aa, synthetic construct
  REGION 1..83: note = Synthetic
MAADGYLPDWLEDTLSEGIRQWWKLKPGPPPPKPNQQHRDDSRGLVLPGYKYLGPFNGLDKGEPVNEADAAALEHDKAYDHQL

SEQ ID NO: 127 - protein, 83 aa, synthetic construct
  REGION 1..83: note = Synthetic
MAADGYLPDWLEDNLSEGIREWWALKPGVPQPKANQQHQDNRRGLVLPGYKYLGPGNGLDKGEPVNEADAAALEHDKAYDQQL

SEQ ID NO: 128 - protein, 83 aa, synthetic construct; identical to SEQ ID NO: 127
  REGION 1..83: note = Synthetic

SEQ ID NO: 129 - protein, 83 aa, synthetic construct; identical to SEQ ID NO: 59
  REGION 1..83: note = Synthetic

SEQ ID NO: 130 - protein, 83 aa, synthetic construct; identical to SEQ ID NO: 59
  REGION 1..83: note = Synthetic

SEQ ID NO: 131 - protein, 83 aa, synthetic construct; identical to SEQ ID NO: 59
  REGION 1..83: note = Synthetic

SEQ ID NO: 132 - protein, 83 aa, synthetic construct
  REGION 1..83: note = Synthetic
MAADGYLPDWLEDTLSEGIRQWWKLRPGPPPPKPAERHKDDSRGLVLPGYKYLGPFNGLDKGEPVNEADAAALEHDKAYDRQL

SEQ ID NO: 133 - protein, 83 aa, synthetic construct; identical to SEQ ID NO: 113
  REGION 1..83: note = Synthetic

SEQ ID NO: 134 - protein, 83 aa, synthetic construct; identical to SEQ ID NO: 59
  REGION 1..83: note = Synthetic

SEQ ID NO: 135 - protein, 82 aa, synthetic construct
  REGION 1..82: note = Synthetic
MSFVDHPPDWLEEVGEGLREFLGLEAGPPKPKPNQQHQDQARGLVLPGYMYLGPGNGLDRGEPVNRADEVAREHDISYNEQL

(19) United States
(12) Patent Application Publication
(10) Pub. No.: US 2025/0066815 A1
(43) Pub. Date: Feb. 27, 2025

(54) ENGINEERED VIRAL CAPSIDS WITH INCREASED STABILITY AND METHODS OF USE THEREOF

(71) Applicant: The Broad Institute, Inc., Cambridge, MA (US)

(72) Inventors: Benjamin Deverman, Cambridge, MA (US); Simon Pacouret, Cambridge, MA (US); Fatmaelzahraa Sobhy Abdelmouty Eid, Cambridge, MA (US)

(21) Appl. No.: 18/950,128

(22) Filed: Nov. 17, 2024

Related U.S. Application Data
(63) Continuation of application No. PCT/US2023/067130, filed on May 17, 2023.
(60) Provisional application No. 63/343,376, filed on May 18, 2022.

(51) Int. Cl. C12N 15/86; C07K 14/005

(52) U.S. Cl. CPC C12N 15/86; C07K 14/005; C12N 2750/14122; C12N 2750/14143

(57) ABSTRACT

Engineered capsid scaffolds comprising one or more modified capsid proteins are described exhibiting properties of improved thermostability while producing at similar levels to the naturally occurring capsid serotype. Embodiments include use and delivery of the engineered capsid scaffolds to allow for increased tolerance for manipulation and mutagenesis.

### CROSS-REFERENCE TO RELATED APPLICATIONS

This application is a continuation application of PCT/US2023/067130, filed May 17, 2023, which claims the benefit of priority to U.S. Provisional Application No. 63/343,376, filed May 18, 2022; the contents of which are incorporated by reference in their entireties herein.

### STATEMENT REGARDING FEDERALLY SPONSORED RESEARCH

This invention was made with government support under Grant No. NS111689 awarded by the National Institutes of Health. The government has certain rights in the invention.

### SEQUENCE LISTING

This application contains a sequence listing filed in electronic form as an xml file entitled BROD-5440US, created on Nov. 15, 2024, and having a size of 217,514 bytes. The contents of the sequence listing are incorporated herein in their entirety.

### TECHNICAL FIELD

The subject matter disclosed herein is generally directed to stabilized capsid scaffold compositions and methods of use.

### BACKGROUND

AAV capsids are widely used in biological applications. For example, AAV9 is widely used as a gene therapy vector. It is currently the only FDA and EMA approved AAV capsid used in a systemically administered gene therapy. Due to the versatility and manufacturability of the AAV9 capsid, it is also being a used as a scaffold for engineering AAV capsids with new properties such as efficient blood-brain-barrier (BBB) crossing and muscle transduction (Deverman et al. CRE-dependent selection yields AAV variants for widespread gene transfer to the adult brain Nat Biotechnol. 2016 34(2):204-9; Chan et al. Engineered AAVs for efficient noninvasive gene delivery to the central and peripheral nervous systems Nat Neurosci. 2017 20(8):1172-1179; Hanlon et al. Selection of an efficient AAV vector for robust CNS transgene expression. Mol Ther Methods Clin Dev. 2019 15:320-332; Nonnenmacher et al. Rapid evolution of blood-brain-barrier-penetrating AAV capsids by RNA-driven biopanning Mol Ther Methods Clin Dev. 2020 20:366-378; Weinmann et al Identification of a myotropic AAV by massively parallel in vivo evaluation of barcoded capsid variants Nat Commun. 2020; 11(1):5432). However, mutations introduced while engineering AAV capsids to retarget their tropism or to introduce resistance to neutralizing antibodies can destabilize the capsid, making manufacturing challenging and may limit the ability of the capsid engineer to introduce multiple beneficial modifications. Improvements to the stability of AAV capsids that allow for engineering of further modifications would be a desirable improvement in the art.

Citation or identification of any document in this application is not an admission that such a document is available as prior art to the present invention.

### SUMMARY

In certain example embodiments, an engineered AAV capsid scaffold is provided comprising one or more modified capsid proteins that improve capsid stability to accept further modifications including insertions that increase the capsid's thermal stability, retarget the capsid's tropism, increase resistance to neutralizing antibodies confer greater mechanical stability, in increase viral titer and/or increase production fitness relative to a reference AAV capsid.

The engineered AAV capsid may comprise one or more modifications that increase thermal stability of the AAV capsid by 1, 2, 3, 4, 5, 6, 7, 8, 9, 10, 11, 12, 13, 14, 15, 16, 17, 18, 19 or 20° C. relative to a reference AAV capsid that does not comprise the one or more modified capsid proteins.

The engineered AAV capsid can be a AAV9 capsid comprising one or more modifications selected from the group consisting of N41D, G56F, V211M, Q233T, D349E, E361Q, D384N, E416T, N419D, K449R, Q456T, V465Q, Y478W, I479L, S483C, P504T, S507T, S508K, W509Y, A510H, E529D, G530D, H584L, Q597N, M640L, D665A, N668A, and E712D of AAV9.

In an embodiment, at least one modification is selected from the group consisting of D384N, S483C, P504T, S508K, and W509Y of AAV9, and at least one modification is selected from the group consisting of E529D, G530D, Y478W, and I479L of AAV9.

In an embodiment, the one or more modifications comprise D384N, S483C, P504T, S508K, and W509Y of AAV9.

In an embodiment, the one or more modifications comprise D384N, S483C, P504T, S508K, W509Y, E529D, and G530D of AAV9.

In an embodiment, the one or more modifications comprise D384N, E529D and G530D of AAV9.

In an embodiment, the one or more modifications comprise N41D, G56F, V211M, Q233T, D384N, Y478W, I479L, S483C, P504T, S508K, W509Y, E529D, G530D, and M640L of AAV9.

In an embodiment, the one or more modifications comprise D384N, S483C, P504T, S508K, W509Y, E529D, and G530D of AAV9.

In an embodiment, the one or more modifications comprise D384N, S483C, P504T, S508K, W509Y, E529D, G530D, and Q597N of AAV9.

In an embodiment, the one or more modifications comprise S483C, P504T, S508K, W509Y, E529D, G530D, and Q597N of AAV9.

In an embodiment, the one or more modifications comprise D384N, S483C, P504T, S508K, W509Y, and Q597N of AAV9.

In an embodiment, the one or more modifications comprise S483C, P504T, S508K, W509Y, and Q597N of AAV9.

In an embodiment, the one or more modifications comprise D384N, S483C, S508K, W509Y, E529D, G530D, and Q597N of AAV9.

In an embodiment, the one or more modifications comprise D384N, S483C, P504T, E529D, G530D, and Q597N of AAV9.

In an embodiment, the one or more modifications comprise D384N, S483C, P504T, W509Y, E529D, G530D, and Q597N of AAV9.

In an embodiment, the one or more modifications comprise Q597N of AAV9.

In an embodiment, the one or more modifications comprise D384N of AAV9.

In an embodiment, the one or more modifications comprise S483C of AAV9.

In an embodiment, the one or more modifications comprise P504T of AAV9.

In an embodiment, the one or more modifications comprise S507T and S508K of AAV9.

In an embodiment, the one or more modifications comprise S507T, S508K, W509Y, and A510H of AAV9.

In an embodiment, the one or more modifications comprise S508K and W509Y of AAV9.

In an embodiment, the one or more modifications comprise Y478W, I479L, E529D, and G530D. In an embodiment, the one or more modifications consist of D384N, S483C, P504T, S508K, W509Y, E529D, and G530D.

In an embodiment, the AAV capsid is an AAV1 capsid and the one or more modifications are selected from the group consisting of E418D, R465Q, S467G, S507T, N510H, I517L, M541L, A568T, F577Y, F584L, H597N, M599Q, N642H, T665A, V699I, and P735N.

In an embodiment, the AAV capsid is a AAV2 capsid and the one or more modifications are selected from the group consisting of S207G, M235L, V372I, N449Q, A467P, I470M, E499N, Y500F, S547A, A590P, V600A, S658P, and T713A.

In an embodiment, the AAV capsid is a AAV3B capsid and the one or more modifications are selected from the group consisting of S205A, Q233T, K310R, V372I, L489V, A505G, S507T, H538S, N540V, E546Q, T548A, T549G, M648L, and T714A.

In an embodiment, the AAV capsid is a AAV6 capsid and the one or more modifications are selected from the group consisting of R465Q, S467G, S507T, N510H, I517L, K531E, M541L, A568T, F577Y, H597N, M599Q, T665A, V699I, and P735N.

In an embodiment, the AAV capsid is a AAV7 capsid and the one or more modifications are selected from the group consisting of V204M, T265S, S388A, S416T, Y466S, G468A, F486Y, K553N, L560M, P569T, F706Y, Q709S, and G711N.

In an embodiment, the AAV capsid is a AAV8 capsid and the one or more modifications are selected from the group consisting of S224N, T415S, G468A, A507G, G508A, A520V, N540S, I542V, N549G, A551G, A555V, S667A, N670A, and S712N.

In an embodiment, the AAV capsid is a AAVrh10 capsid and the one or more modifications are selected from the group consisting of S224N, N263S, E330D, K333T, I343V, Q417T, T493K, S559N, A591T, V595T, D659N, L669F, and T722V.

In an embodiment, the AAV capsid is a AAVrh8 capsid and the one or more modifications are selected from the group consisting of L389V, V479L, A507T, F509Y, K510H, D531E, G552N, L559M, A568T, N584L, H597N, I602L, and N668A.

In an embodiment, the engineered AAV capsid comprises at least one n-mer insertion that re-targets or improves transducibility of the engineered AAV capsid. In one example embodiment, the n-mer consists of a 7-mer. In one example embodiment, the n-mer is inserted at amino acid 588 of AAV9 VP1 or an analogous position of AAV1, AAV2, AAV3B, AAV6, AAV7, AAV8, AAVrh10, AAVrh8, or AAV-PHP.eB

In another aspect, example embodiments disclosed herein include vector systems comprising one or more vectors encoding an engineered AAV capsid of the present invention. Compositions comprising the engineered AAV capsid disclosed herein are also provided.

Methods of delivering cargos to cells are provided, comprising the steps of administering an engineered AVV particle as disclosed herein to a population of cells, the engineered AAV particle further loaded with a cargo (including a recombinant AAV genome encoding a payload such as a therapeutic polypeptide or nucleic acid) and optionally further comprising on or more n-mer motifs inserted into the capsid that determine a tropism of the engineered AAV particle.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1F: Impact of peptide display on capsid thermal stability. 7-mer insertion at VP3 position 588 strongly destabilizes the AAV9 capsid. (A-D) DSF derivative profiles obtained for (A) two AAV9 preparations, (B) three AAVPHP.eB preparations, (C) three AAV.BI28 preparations or (D) all eight AAV preparations. (E) Summary of the melting temperatures measured for the eight AAV preparations. (F) Summary of the melting temperatures measured by Bennett and colleagues for 10 naturally-occurring AAV serotypes (Bennett, A. et al. (2017), Mol. Ther.—Methods Clin. Dev. 6, 171-182.

FIG. 2A-2C: Selection of target residues for consensus mutagenesis. (A) Multiple sequence alignment of the VP1 protein of 75 naturally occurring AAV serotypes (Zinn, E. et al. (2015), Cell. Rep. 12, 1056-1058) generated with T-coffee (Notredame, C. (2000), J. Mol. Biol. 302, 205-217) (SEQ ID NO: 59-135). The top, bolded consensus sequence was calculated from this MSA, using a conservation threshold of 85%. The MSA was truncated for visualization purposes. (B) List of the 15 divergent residues identified between AAV9 VP1 and the consensus template. (C) Target residues, represented at the outer surface of AAV9 capsid, in the region of the 3-fold symmetry axis.

FIG. 3A-3C: Consensus Mutagenesis can be used to increase the thermal stability of AAV9. (A) Summary of the 14 divergent residues between AAV9 and AAVTS. (B) Derivative DSF profiles obtained for AAV9 (blue) and AAVTS (pink). (C) Melting temperatures measured from the DSF experiments for AAV9 and AAVTS.

FIG. 4A-4D: Consensus mutagenesis increases the thermal stability of AAV9 and AAV-PHP.eB without affecting production titers and VP stoichiometry. (A) Production titers measured from clarified the cell lysates of AAV9, AAVTS9, AAV-PHP.eB and AAVTS-PHP.eB (n=3). (B) SDS-PAGE analysis of AAV9, AAVTS9, AAV-PHP.eB and AAVTS-PHP.eB. (C) Derivative DSF profiles obtained for AAV9 (dark gray) and AAVTS (gray), AAV-PHP.eB (cyan) and AAVTS-PHP.eB (green) (D) Melting temperatures measured from the DSF experiments for AAV9, AAVTS9, AAV-PHP.eB and AAVTS-PHP.eB.

FIG. 5A-5C: Thermal stability enhancements are independent of transgene and purification method. (A) Derivative DSF profiles obtained for AAV9 (blue), AAV-PHP.eB (cyan), AAVTS9 (pink), and AAVTS-PHP.eB (green) preparations purified by POROS AAV9 capture affinity chromatography (B) Derivative DSF profiles obtained for AAV9 (blue), AAV-PHP.eB (cyan), AAVTS9 (pink), and AAVTS-PHP.eB (green) preparations purified by Iodixanol gradient ultracentrifugation. (C) Melting temperatures measured from the DSF experiments run with POROS and Iodixanol-purified AAV preparations.

FIG. 6A-6B: The AAVTS9 and AAVTS-PHP.eB mutations retain or improve in vitro transduction. (A) eGFP fluorescence images of HEK293 cells, taken 48 h post infection with AAV9, AAVTS9, AAV-PHP.eB or AAVTS-PHP.eB (MOI=5E4). (B) Luminescence levels, measured from HEK293 cells, 48 h post infection with the same four AAV serotypes, at a MOI of either 5E4, 5E3 or 5E2.

FIG. 7: Impact of stabilizing mutations on in vivo transduction. EGFP fluorescence images of C57 mouse liver slices, harvested 4 weeks post retro-orbital administration of AAV9, AAVTS9, AAV-PHP.eB or AAVTS-PHP.eB vectors, packaging a CAG-eGFP-P2A-Luciferase-SV40-WPRE transgenes (1E11 vg/animal).

FIG. 8A-8B: Impact of stabilizing mutations on in vivo transduction of the brain. EGFP fluorescence images of C57 mouse brain slices, harvested 4 weeks post retro-orbital administration of AAV9, AAVTS9, AAV-PHP.eB or AAVTS-PHP.eB vectors, packaging a CAG-eGFP-P2A-Luciferase SV40-WPRE transgenes (1E11 vg/animal). (A) exposure time=80 ms. (B) exposure time=1000 ms.

FIG. 9A-9B: Impact of stabilizing mutations on in vivo transduction. EGFP fluorescence images of C57 mouse brain slices, harvested 4 weeks post retro-orbital administration of AAV9, AAVTS9, AAV-PHP.eB or AAVTS-PHP.eB vectors, packaging a CAG-NLS-GFP-WPRE transgenes (1E11 vg/animal). (A) exposure time=80 ms. (B) exposure time=1000 ms.

FIG. 10A-10E: Impact of individual stabilizing mutations on viral titers and thermal stability. (A) Small scale production titers, measured by ddPCR from the clarified lysates of individual mutant producing cells. (B-C) Derivative DSF profiles and resulting melting temperatures obtained for individual mutant and control preparations. (D) SDS-PAGE analysis of variant preparation purity. Each purified sample was loaded at 5 uL per well, and the gel was stained with SYPRO Ruby. (E) Summary of the viral titers and melting temperatures obtained for all variants.

FIG. 11A-11E: Generation of minimally altered AAV9 scaffolds with enhanced thermal stability. (A)) Mutated residues in AAVTS9.2.1, AAVTS9.2.2 and AAVTS9.2.3, represented at the outer surface of the AAV9 capsid. (B) Viral titers measured in the cell lysate by ddPCR. (C) SDS-PAGE analysis of variant preparation purity. Each purified sample was loaded at 1E10 vg per well, and the gel was stained with SYPRO Ruby. (D) Derivative DSF profiles obtained for AAV9, AAVTS9, AAVTS9.2.1, AAVTS9.2.2 and AAVTS9.2.3. (E) Luminescence levels, measured from HEK293 cells, 48 h post infection with the same five AAV serotypes, at a MOI of either 5E4, 5E3 or 5E2.

FIG. 12A-12F: Comparative analysis of AAV9 and AAVTS9 evolvability. (A) Overview on experimental procedure. A library of 150 k 7-mer peptides was inserted into the cap gene of AAV9 and AAVTS9, at amino acid position 588. The resulting AAV libraries were produced, purified and subjected to NGS. (B) SDS-PAGE analysis of library sample purity. (C) Total turbonuclease-resistant viral genome (vg) yields, quantified by ddPCR in the cell lysates and purified library preparations. (D) Derivative DSF profiles obtained for individual vector (top) and library (bottom) preparations. (E) Distribution of the log 2enrichment scores obtained at the nucleotide level for AAV9 and AAVTS9. (F) Pairwise density plot of the log 2enrichment scores of both libraries, at the nucleotide level.

FIG. 13A-13B: Other serotypes can be subjected to consensus mutagenesis. (A-B) Number of consensus mutations present in additional AAV serotypes, plotted as a function of the conservation threshold used for residue identification. (A) Consensus thresholds ranging from 0-100. (B) Zoom on consensus thresholds ranging between 50-100.

FIG. 14A-14D: Most engineered AAV capsid variants are destabilized relatively to AAV9. (A) DSF profile obtained for four AAV9 and four AAV-PHP.eB vector preparations. (B) Melting temperatures measured for the eight preparations. (C) Capsid Melting Temperature of naturally occurring AAV serotypes (Bennett et al, 2017). (D) Capsid melting temperatures of 153 AAV9-derived capsid variants, ranked by decreasing Tm. Each Tm assay was run with an AAV control prep.

FIG. 15: List of consensus residues identified from multiple sequence alignment of 75 serotypes shown in FIG. 2A.

FIG. 16A-16D: Consensus mutagenesis of AAV9 yields 7 functional, stabilized AAV variants producing near AAV9 levels. (A) Mutants selected for DSF, ddPCR and in vitro transduction assays. (B) Capsid melting temperature of selected mutants, measured by DSF. (C) Crude lysate viral titers of selected mutants, measured by ddPCR and normalized to AAV9 titers (n=3). (D) Luminescence levels measured from HEK293T cell lysates, 2 days following transduction, and normalized to AAV9 levels.

FIG. 17A-17E: Combining stabilizing mutations yields highly stable capsids compatible with 7mer peptide insertion in VR VIII. (A) Mutants selected for thermal stability and production assays. Each mutant was produced and purified in the absence or presence of the PHP.eB peptide. (B) SDS-PAGE analysis of purified mutant preparations (1E9 vg/well). (C) Viral titers of selected mutants, measured in crude lysates by ddPCR and normalized to AAV9 titers (n=3). (D) Capsid melting temperatures of selected mutants, measured by DSF. (E) Correlation between capsid melting temperatures measured with or without the PHP.eB peptide.

FIG. 18A-18F: Study of the impact of consensus residue interactions on the thermal stability and function of AAV9. (A-C) Variable residues of AAV9TS11 (A), AAV9TS2 (B) and AAV9TS12 (C) represented at the surface of the AAV9 capsid. (D) DSF signal obtained for the different (de)stabilized variants produced and characterized in this experiment. (E) Melting temperatures measured from the DSF experiment.

FIG. 19A-19B: TS mutations also stabilize other low Tm engineered variants such as AAV-BI28. (A) Derivative DSF profiles obtained for AAV9, AAV-BI28, AAV9TS1-BI28, and AAV9TS2-BI28 preparations, packaging a mScarlet-P2A-Luciferase dual reporter transgene cassette. (B) Melting temperatures measured from the DSF experiment.

FIG. 20A-20C: Impact of transgene and purification method on thermal stability data. (A) Derivative DSF profiles obtained for AAV9, AAV-PHP.eB, AAV9TS1, and AAV9TS1-PHP.eB preparations purified by POROS AAV9 capture affinity chromatography. (B) Derivative DSF profiles obtained for AAV9, AAV-PHP.eB, AAV9TS1, and AAV9TS1-PHP.eB preparations purified by Iodixanol gradient ultracentrifugation. (C) Melting temperatures measured from the DSF experiments run with POROS and Iodixanol-purified AAV preparations.

FIG. 21: TS mutations stabilize low Tm capsid variants at various acidic pH. Melting temperatures of AAV9, AAV-PHP.eB, AAV9TS2, AAV9TS2-PHP.eB plotted as a function of the pH in 0.1M sodium acetate.

FIG. 22A-22F: In vitro and in vivo function of TS mutants. (A-C) Luminescence levels measured from HEK293T (A), hCMEC (B), or CHO (C) cell lysates, 2 days post transduction (MOI=5000, 6667 and 5000, respectively) with AAVs packaging a dual reporter CAG-GFP-P2A-Luciferase. (D) Images of native GFP fluorescence from sagittal brain sections of animals injected with 1E11 vg of AAV9 or AAV9TS1-AAV9TS10, packaging a dual reporter GFP-P2A-Luciferase. (E) Images of native GFP fluorescence from liver sections of C57 mice injected with 1e11 vg of AAV9, AAV-PHP.eB, AAV9TS1-AAV9TS10 or AAV9TS1-PHP.eB to AAV9T10-PHP.eB, packaging a dual reporter CAG-GFP-P2A-Luciferase. (F) Images of native GFP fluorescence from sagittal brain sections of C57 mice injected with 1e11 vg of AAV9, AAV-PHP.eB, AAV9TS1-AAV9TS10 or AAV9TS1-PHP.eB to AAV9T10-PHP.eB, packaging a dual reporter CAG-GFP-P2A-Luciferase.

FIG. 23A-23B: Impact of TS mutations on in vivo transduction. (A-B) GFP fluorescence images of C57 mouse brain slices, harvested 4 weeks post retro-orbital administration of AAV9, AAV9TS1, AAV-PHP.eB or AAV9TS1-PHP.eB vectors, packaging a CAG-NLS-GFP-WPRE transgene (1E11 vg/animal). (A) exposure time=80 ms. (B) exposure time=1000 ms.

FIG. 24A-24I: The AAV9TS1 capsid is amenable to directed evolution with 7-mer library peptide insertion in loop VIII. (A) Schematic of library cloning, production, purification, and characterization. (B) SDS-PAGE analysis of viral library purity. 1E10 vg of virus library and virus controls 1-3 were loaded into each well. The gel was stained with SYPRO Ruby. (C) Library viral titers, measured by duplex ddPCR with cap-specific primers and probes (AAV9-FAM and AAV9TS1-HEX). (D) DSF profile of viral library. (E) Genome release profile of viral library, measured by duplex ddPCR following incubation at increasing temperatures. (F) Distribution of the log 2 enrichment production fitness scores obtained at the nucleotide level for AAV9 and AAV9TS1 variants. (G) Pairwise density plot of the log 2 enrichment production fitness scores of both libraries, at the nucleotide level. (H) Distribution of the log 2 enrichment HEK293Ttransduction scores obtained at the nucleotide level for AAV9 and AAV9TS1 variants. (I) Pairwise density plot of the log 2 enrichment HEK293Ttransduction scores of both libraries, at the nucleotide level.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide an engineered AAV capsid comprising one or more modified capsid proteins that improve capsid stability. The present disclosure provides novel engineered AAV capsids with improved stability that allow for further modifications without diminishing desirable properties of the capsid. Consensus mutagenesis across AAV serotypes was used to generate improved variant capsid proteins. Advantageously, the variant capsid proteins have increased thermal stability while maintaining or improving transduction efficiency and packaging, and without affecting VP1-3 stoichiometry and viral titers. Additionally, the improvements in thermal stability are independent of cargo. Improved stability allows for further modifications such as insertion of motifs that modify capsid tropism and other multiple beneficial modifications.

Methods of identifying and making the variant capsids with improved thermal stability are also provided. In an embodiment, the engineered AAV capsid provides improved capsid stability that allows the engineered AAV capsid to accept further modifications including insertions, substitutions, or deletions that retarget the capsid's tropism. In an embodiment, engineering AAV capsids with one or more of the mutations described below can improve the thermostability of the capsid, make the capsid more tolerant of additional mutagenesis, or both. In one aspect, the increased stability of the capsid can provide the engineered AAV capsid protein with increased tolerance of higher processing or storage that can improve methods of use, including in the delivery of cargos to cells using the engineered AAV particle loaded with a cargo.

### Engineered Viral Capsids Having Increased Stability

Described herein are various embodiments of engineered viral capsids. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle). The engineered viral capsids described herein can include one or more engineered viral capsid proteins described herein.

The engineered viral capsids can be variants of a wild-type viral capsid. For example, the engineered AAV capsids can be variants of wild-type AAV capsids as provided in Table 1. The wild-type AAV capsids can be composed of VP1, VP2, VP3 capsid proteins or a combination thereof (including in a 1:1:10 ratio). In other words, the engineered AAV capsids may comprise a modified capsid protein, for example, a modified VP1, modified VP2, and/or modified VP3 capsid proteins. The serotype of the reference wild-type (naturally-occurring) AAV capsid can be AAV1, AAV2, AAV3, AAV4, AAV5, AAV6, AAV7, AAV8, AAV9, AAV-PHP.eB AAVrh.74, AAVrh 8, AAVrh10 or any combination thereof. In one embodiment, the serotype of the wild-type AAV capsid can be AAV9. In one embodiment, the AAV capsid to be modified can be AAV-PHP.eB. The engineered AAV capsids can have a different tropism than that of the reference wild-type AAV capsid.

TABLE 1

| AAV | Accession | |
|---|---|---|
| Serotype | No. | Sequence |
| AAV9 | | MAADGYLPDWLEDNLSEGIREWWALKPGAPQPKANQQHQDNARGLVLPGYKYLGPG |
| | | NGLDKGEPVNAADAAALEHDKAYDQQLKAGDNPYLKYNHADAEFQERLKEDTSFGGN |
| | | LGRAVFQAKKRLLEPLGLVEEAAKTAPGKKRPVEQSPQEPDSSAGIGKSGAQPAKKRLN |
| | | FGQTGDTESVPDPQPIGEPPAAPSGVGSLTMASGGGAPVADNNEGADGVGSSSGNWHC |
| | | DSQWLGDRVITTSTRTWALPTYNNHLYKQISNSTSGGSSNDNAYFGYSTPWGYFDFNRF |
| | | HCHFSPRDWQRLINNNWGFRPKRLNFKLFNIQVKEVTDNNGVKTIANNLTSTVQVFTDS |
| | | DYQLPYVLGSAHEGCLPPFPADVFMIPQYGYLTLNDGSQAVGRSSFYCLEYFPSQMLRT |
| | | GNNFQFSYEFENVPFHSSYAHSQSLDRLMNPLIDQYLYYLSKTINGSGQNQQTLKFSVAG |
| | | PSNMAVQGRNYIPGPSYRQQRVSTTVTQNNNSEFAWPGASSWALNGRNSLMNPGPAM |
| | | ASHKEGEDRFFPLSGSLIFGKQGTGRDNVDADKVMITNEEEIKTTNPVATESYGQVATN |
| | | HQSAQAQAQTGWVQNQGILPGMVWQDRDVYLQGPIWAKIPHTDGNFHPSPLMGGFGM |
| | | KHPPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL* (SEQ ID NO: 1) |
| AAV9 | | ATGGCTGCCGATGGTTATCTTCCAGATTGGCTCGAGGACAACCTTAGTGAAGGAATT |
| | | CGCGAGTGGTGGGCTTTGAAACCTGGAGCCCCTCAACCCAAGGCAAATCAACAACA |
| | | TCAAGACAACGCTCGAGGTCTTGTGCTTCCGGGTTACAAATACCTTGGACCCGGCAA |
| | | CGGACTCGACAAGGGGGAGCCGGTCAACGCAGCAGACGCGGCGGCCCTCGAGCACG |
| | | ACAAGGCCTACGACCAGCAGCTCAAGGCCGGAGACAACCCGTACCTCAAGTACAAC |
| | | CACGCCGACGCCGAGTTCCAGGAGCGGCTCAAAGAAGATACGTCTTTTGGGGGCAA |
| | | CCTCGGGCGAGCAGTCTTCCAGGCCAAAAAGAGGCTTCTTGAACCTCTTGGTCTGGT |
| | | TGAGGAAGCGGCTAAGACGGCTCCTGGAAAGAAGAGGCCTGTAGAGCAGTCTCCTC |
| | | AGGAACCGGACTCCTCCGCGGGTATTGGCAAATCGGGTGCACAGCCCGCTAAAAAG |
| | | AGACTCAATTTCGGTCAGACTGGCGACACAGAGTCAGTCCCAGACCCTCAACCAATC |
| | | GGAGAACCTCCCGCAGCCCCCTCAGGTGTGGGATCTCTTACAATGGCTTCAGGTGGT |
| | | GGCGCACCAGTGGCAGACAATAACGAAGGTGCCGATGGAGTGGGTAGTTCCTCGGG |
| | | AAATTGGCATTGCGATTCCCAATGGCTGGGGGACAGAGTCATCACCACCAGCACCCG |
| | | AACCTGGGCCCTGCCCACCTACAACAATCACCTCTACAAGCAAATCTCCAACAGCAC |
| | | ATCTGGAGGATCTTCAAATGACAACGCCTACTTCGGCTACAGCACCCCCTGGGGGTA |
| | | TTTTGACTTCAACAGATTCCACTGCCACTTCTCACCACGTGACTGGCAGCGACTCATC |
| | | AACAACAACTGGGGATTCCGGCCTAAGCGACTCAACTTCAAGCTCTTCAACATTCAG |
| | | GTCAAAGAGGTTACGGACAACAATGGAGTCAAGACCATCGCCAATAACCTTACCAG |
| | | CACGGTCCAGGTCTTCACGGACTCAGACTATCAGCTCCCGTACGTGCTCGGGTCGGC |
| | | TCACGAGGGCTGCCTCCCGCCGTTCCCAGCGGACGTTTTCATGATTCCTCAGTACGG |
| | | GTATCTGACGCTTAATGATGGAAGCCAGGCCGTGGGTCGTTCGTCCTTTTACTGCCTG |
| | | GAATATTTCCCGTCGCAAATGCTAAGAACGGGTAACAACTTCCAGTTCAGCTACGAG |
| | | TTTGAGAACGTACCTTTCCATAGCAGCTACGCTCACAGCCAAAGCCTGGACCGACTA |
| | | ATGAATCCACTCATCGACCAATACTTGTACTATCTCTCAAAGACTATTAACGGTTCTG |
| | | GACAGAATCAACAAACGCTAAAATTCAGTGTGGCCGGACCCAGCAACATGGCTGTC |
| | | CAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCT |
| | | CAATGGACGTAATAGCTTGATGAATCCTGGACCTGCTATGGCCAGCCACAAAGAAG |
| | | GAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAA |
| | | ACTACTAACCCGGTAGCAACGGAGTCCTATGGACAAGTGGCCACAAACCACCAGAG |
| | | TGCCCAAGCACAGGCGCAGACCGGCTGGGTTCAAAACCAAGGAATACTTCCGGGTA |
| | | TGGTTTGGCAGGACAGAGATGTGTACCTGCAAGGACCCATTTGGGCCAAAATTCCTC |
| | | ACACGGACGGCAACTTTCACCCTTCTCCGCTGATGGGAGGGTTTGGAATGAAGCACC |
| | | CGCCTCCTCAGATCCTCATCAAAAACACACCTGTACCTGCGGATCCTCCAACGGCCT |
| | | TCAACAAGGACAAGCTGAACTCTTTCATCACCCAGTATTCTACTGGCCAAGTCAGCG |
| | | TGGAGATCGAGTGGGAGCTGCAGAAGGAAAACAGCAAGCGCTGGAACCCGGAGAT |
| | | CCAGTACACTTCCAACTATTACAAGTCTAATAATGTTGAATTTGCTGTTAATACTGAA |
| | | GGTGTATATAGTGAACCCCGCCCCATTGGCACCAGATACCTGACTCGTAATCTGTAA |
| | | (SEQ ID NO: 2) |
| AAV-PHP.eB | | MAADGYLPDWLEDNLSEGIREWWALKPGAPQPKANQQHQDNARGLVLPGYKYLGPG |
| | | NGLDKGEPVNAADAAALEHDKAYDQQLKAGDNPYLKYNHADAEFQERLKEDTSFGGN |
| | | LGRAVFQAKKRLLEPLGLVEEAAKTAPGKKRPVEQSPQEPDSSAGIGKSGAQPAKKRLN |
| | | FGQTGDTESVPDPQPIGEPPAAPSGVGSLTMASGGGAPVADNNEGADGVGSSSGNWHC |
| | | DSQWLGDRVITTSTRTWALPTYNNHLYKQISNSTSGGSSNDNAYFGYSTPWGYFDFNRF |
| | | HCHFSPRDWQRLINNNWGFRPKRLNFKLFNIQVKEVTDNNGVKTIANNLTSTVQVFTDS |
| | | DYQLPYVLGSAHEGCLPPFPADVFMIPQYGYLTLNDGSQAVGRSSFYCLEYFPSQMLRT |
| | | GNNFQFSYEFENVPFHSSYAHSQSLDRLMNPLIDQYLYYLSRTINGSGQNQQTLKFSVAG |
| | | PSNMAVQGRNYIPGPSYRQQRVSTTVTQNNNSEFAWPGASSWALNGRNSLMNPGPAM |
| | | ASHKEGEDRFFPLSGSLIFGKQGTGRDNVDADKVMITNEEEIKTTNPVATESYGQVATN |
| | | HQSDGTLAVPFKAQAQTGWVQNQGILPGMVWQDRDVYLQGPIWAKIPHTDGNFHPSPL |
| | | MGGFGMKHPPPQILIKNTPVPADPPTAFNKDKLNSFITQYSTGQVSVEIEWELQKENSKR |
| | | WNPEIQYTSNYYKSNNVEFAVNTEGVYSEPRPIGTRYLTRNL* (SEQ ID NO: 3) |
| AAV-PHP.eB | | ATGGCTGCCGATGGTTATCTTCCAGATTGGCTCGAGGACAACCTTAGTGAAGGAATT |
| | | CGCGAGTGGTGGGCTTTGAAACCTGGAGCCCCTCAACCCAAGGCAAATCAACAACA |
| | | TCAAGACAACGCTCGAGGTCTTGTGCTTCCGGGTTACAAATACCTTGGACCCGGCAA |
| | | CGGACTCGACAAGGGGGAGCCGGTCAACGCAGCAGACGCGGCGGCCCTCGAGCACG |
| | | ACAAGGCCTACGACCAGCAGCTCAAGGCCGGAGACAACCCGTACCTCAAGTACAAC |
| | | CACGCCGACGCCGAGTTCCAGGAGCGGCTCAAAGAAGATACGTCTTTTGGGGGCAA |
| | | CCTCGGGCGAGCAGTCTTCCAGGCCAAAAAGAGGCTTCTTGAACCTCTTGGTCTGGT |
| | | TGAGGAAGCGGCTAAGACGGCTCCTGGAAAGAAGAGGCCTGTAGAGCAGTCTCCTC |
| | | AGGAACCGGACTCCTCCGCGGGTATTGGCAAATCGGGTGCACAGCCCGCTAAAAAG |
| | | AGACTCAATTTCGGTCAGACTGGCGACACAGAGTCAGTCCCAGACCCTCAACCAATC |
| | | GGAGAACCTCCCGCAGCCCCCTCAGGTGTGGGATCTCTTACAATGGCTTCAGGTGGT |
| | | GGCGCACCAGTGGCAGACAATAACGAAGGTGCCGATGGAGTGGGTAGTTCCTCGGG |
| | | AAATTGGCATTGCGATTCCCAATGGCTGGGGGACAGAGTCATCACCACCAGCACCCG |
| | | AACCTGGGCCCTGCCCACCTACAACAATCACCTCTACAAGCAAATCTCCAACAGCAC |
| | | ATCTGGAGGATCTTCAAATGACAACGCCTACTTCGGCTACAGCACCCCCTGGGGGTA |
| | | TTTTGACTTCAACAGATTCCACTGCCACTTCTCACCACGTGACTGGCAGCGACTCATC |
| | | AACAACAACTGGGGATTCCGGCCTAAGCGACTCAACTTCAAGCTCTTCAACATTCAG |
| | | GTCAAAGAGGTTACGGACAACAATGGAGTCAAGACCATCGCCAATAACCTTACCAG |
| | | CACGGTCCAGGTCTTCACGGACTCAGACTATCAGCTCCCGTACGTGCTCGGGTCGGC |
| | | TCACGAGGGCTGCCTCCCGCCGTTCCCAGCGGACGTTTTCATGATTCCTCAGTACGG |
| | | GTATCTGACGCTTAATGATGGAAGCCAGGCCGTGGGTCGTTCGTCCTTTTACTGCCTG |
| | | GAATATTTCCCGTCGCAAATGCTAAGAACGGGTAACAACTTCCAGTTCAGCTACGAG |
| | | TTTGAGAACGTACCTTTCCATAGCAGCTACGCTCACAGCCAAAGCCTGGACCGACTA |
| | | ATGAATCCACTCATCGACCAATACTTGTACTATCTCTCTAGAACTATTAACGGTTCTG |
| | | GACAGAATCAACAAACGCTAAAATTCAGTGTGGCCGGACCCAGCAACATGGCTGTC |
| | | CAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCT |
| | | CAATGGACGTAATAGCTTGATGAATCCTGGACCTGCTATGGCCAGCCACAAAGAAG |
| | | GAGAGGACCGTTTCTTTCCTTTGTCTGGATCTTTAATTTTTGGCAAACAAGGTACCGG |
| | | CAGAGACAACGTGGATGCGGACAAAGTCATGATAACCAACGAAGAAGAAATTAAA |
| | | ACTACTAACCCGGTAGCAACGGAGTCCTATGGACAAGTGGCCACAAACCACCAGAG |
| | | TGATGGGACTTTGGCGGTGCCTTTTAAGGCACAGGCGCAGACCGGTTGGGTTCAAAA |
| | | CCAAGGAATACTTCCGGGTATGGTTTGGCAGGACAGAGATGTGTACCTGCAAGGAC |
| | | CCATTTGGGCCAAAATTCCTCACACGGACGGCAACTTTCACCCTTCTCCGCTGATGG |
| | | GAGGGTTTGGAATGAAGCACCCGCCTCCTCAGATCCTCATCAAAAACACACCTGTAC |
| | | CTGCGGATCCTCCAACGGCCTTCAACAAGGACAAGCTGAACTCTTTCATCACCCAGT |
| | | ATTCTACTGGCCAAGTCAGCGTGGAGATCGAGTGGGAGCTGCAGAAGGAAAACAGC |
| | | AAGCGCTGGAACCCGGAGATCCAGTACACTTCCAACTATTACAAGTCTAATAATGTT |
| | | GAATTTGCTGTTAATACTGAAGGTGTATATAGTGAACCCCGCCCCATTGGCACCAGA |
| | | TACCTGACTCGTAATCTG (SEQ ID NO: 4) |
| AAV1 | AAD27757.1 | MAADGYLPDWLEDNLSEGIREWWDLKPGAPKPKANQQKQDDGRGLVLPGYKYLGPFN |
| | | GLDKGEPVNAADAAALEHDKAYDQQLKAGDNPYLRYNHADAEFQERLQEDTSFGGNL |
| | | GRAVFQAKKRVLEPLGLVEEGAKTAPGKKRPVEQSPQEPDSSSGIGKTGQQPAKKRLNF |
| | | GQTGDSESVPDPQPLGEPPATPAAVGPTTMASGGGAPMADNNEGADGVGNASGNWHC |
| | | DSTWLGDRVITTSTRTWALPTYNNHLYKQISSASTGASNDNHYFGYSTPWGYFDFNRFH |
| | | CHFSPRDWQRLINNNWGFRPKRLNFKLFNIQVKEVTTNDGVTTIANNLTSTVQVFSDSE |
| | | YQLPYVLGSAHQGCLPPFPADVFMIPQYGYLTLNNGSQAVGRSSFYCLEYFPSQMLRTG |
| | | NNFTFSYTFEEVPFHSSYAHSQSLDRLMNPLIDQYLYYLNRTQNQSGSAQNKDLLFSRGS |
| | | PAGMSVQPKNWLPGPCYRQQRVSKTKTDNNNSNFTWTGASKYNLNGRESIINPGTAMA |
| | | SHKDDEDKFFPMSGVMIFGKESAGASNTALDNVMITDEEEIKATNPVATERFGTVAVNF |
| | | QSSSTDPATGDVHAMGALPGMVWQDRDVYLQGPIWAKIPHTDGHFHPSPLMGGFGLNP |
| | | PPQILIKNTPVPANPPAEFSATKFASFITQYSTGQVSVEIEWELQKENSKRWNPEVQYTSN |
| | | YAKSANVDFTVDNNGLYTEPRPIGTRYLTRPL* (SEQ ID NO: 5) |
| AAV1 | AAD27757.1 | ATGGCTGCCGATGGTTATCTTCCAGATTGGCTCGAGGACAACCTCTCTGAGGGCATT |
| | | CGCGAGTGGTGGGACTTGAAACCTGGAGCCCCGAAGCCCAAAGCCAACCAGCAAAA |
| | | GCAGGACGACGGCCGGGGTCTGGTGCTTCCTGGCTACAAGTACCTCGGACCCTTCAA |
| | | CGGACTCGACAAGGGGGAGCCCGTCAACGCGGCGGACGCAGCGGCCCTCGAGCACG |
| | | ACAAGGCCTACGACCAGCAGCTCAAAGCGGGTGACAATCCGTACCTGCGGTATAAC |
| | | CACGCCGACGCCGAGTTTCAGGAGCGTCTGCAAGAAGATACGTCTTTTGGGGGCAAC |
| | | CTCGGGCGAGCAGTCTTCCAGGCCAAGAAGCGGGTTCTCGAACCTCTCGGTCTGGTT |
| | | GAGGAAGGCGCTAAGACGGCTCCTGGAAAGAAACGTCCGGTAGAGCAGTCGCCACA |
| | | AGAGCCAGACTCCTCCTCGGGCACGGCAAGACAGGCCAGCAGCCCGCTAAAAAGAG |
| | | ACTCAATTTTGGTCAGACTGGCGACTCAGAGTCAGTCCCCGATCCACAACCTCTCGG |
| | | AGAACCTCCAGCAACCCCCGCTGCTGTGGGACCTACTACAATGGCTTCAGGCGGTGG |
| | | CGCACCAATGGCAGACAATAACGAAGGCGCCGACGGAGTGGGTAATGCCTCAGGAA |
| | | ATTGGCATTGCGATTCCACATGGCTGGGCGACAGAGTCATCACCACCAGCACCCGCA |
| | | CCTGGGCCTTGCCCACCTACAATAACCACCTCTACAAGCAAATCTCCAGTGCTTCAA |
| | | CGGGGGCCAGCAACGACAACCACTACTTCGGCTACAGCACCCCCTGGGGGTATTTTG |
| | | ATTTCAACAGATTCCACTGCCACTTTTCACCACGTGACTGGCAGCGACTCATCAACA |
| | | ACAATTGGGGATTCCGGCCCAAGAGACTCAACTTCAAACTCTTCAACATCCAAGTCA |
| | | AGGAGGTCACGACGAATGATGGCGTCACAACCATCGCTAATAACCTTACCAGCACG |
| | | GTTCAAGTCTTCTCGGACTCGGAGTACCAGCTTCCGTACGTCCTCGGCTCTGCGCACC |
| | | AGGGCTGCCTCCCTCCGTTCCCGGCGGACGTGTTCATGATTCCGCAATACGGCTACC |
| | | TGACGCTCAACAATGGCAGCCAAGCCGTGGGACGTTCATCCTTTTACTGCCTGGAAT |
| | | ATTTCCCTTCTCAGATGCTGAGAACGGGCAACAACTTTACCTTCAGCTACACCTTTGA |
| | | GGAAGTGCCTTTCCACAGCAGCTACGCGCACAGCCAGAGCCTGGACCGGCTGATGA |
| | | ATCCTCTCATCGACCAATACCTGTATTACCTGAACAGAACTCAAAATCAGTCCGGAA |
| | | GTGCCCAAAACAAGGACTTGCTGTTTAGCCGTGGGTCTCCAGCTGGCATGTCTGTTC |
| | | AGCCCAAAAACTGGCTACCTGGACCCTGTTATCGGCAGCAGCGCGTTTCTAAAACAA |
| | | AAACAGACAACAACAACAGCAATTTTACCTGGACTGGTGCTTCAAAATATAACCTCA |
| | | ATGGGCGTGAATCCATCATCAACCCTGGCACTGCTATGGCCTCACACAAAGACGACG |
| | | AAGACAAGTTCTTTCCCATGAGCGGTGTCATGATTTTTGGAAAAGAGAGCGCCGGAG |
| | | CTTCAAACACTGCATTGGACAATGTCATGATTACAGACGAAGAGGAAATTAAAGCC |
| | | ACTAACCCTGTGGCCACCGAAAGATTTGGGACCGTGGCAGTCAATTTCCAGAGCAGC |
| | | AGCACAGACCCTGCGACCGGAGATGTGCATGCTATGGGAGCATTACCTGGCATGGT |
| | | GTGGCAAGATAGAGACGTGTACCTGCAGGGTCCCATTTGGGCCAAAATTCCTCACAC |
| | | AGATGGACACTTTCACCCGTCTCCTCTTATGGGCGGCTTTGGACTCAAGAACCCGCC |
| | | TCCTCAGATCCTCATCAAAAACACGCCTGTTCCTGCGAATCCTCCGGCGGAGTTTTCA |
| | | GCTACAAAGTTTGCTTCATTCATCACCCAATACTCCACAGGACAAGTGAGTGTGGAA |
| | | ATTGAATGGGAGCTGCAGAAAGAAAACAGCAAGCGCTGGAATCCCGAAGTGCAGTA |
| | | CACATCCAATTATGCAAAATCTGCCAACGTTGATTTTACTGTGGACAACAATGGACT |
| | | TTATACTGAGCCTCGCCCCATTGGCACCCGTTACCTTACCCGTCCCCTGTAA (SEQ ID |
| | | NO: 6) |
| AAV2 | AAC03780.1 | MAADGYLPDWLEDTLSEGIRQWWKLKPGPPPPKPAERHKDDSRGLVLPGYKYLGPFNG |
| | | LDKGEPVNEADAAALEHDKAYDRQLDSGDNPYLKYNHADAEFQERLKEDTSFGGNLG |
| | | RAVFQAKKRVLEPLGLVEEPVKTAPGKKRPVEHSPVEPDSSSGTGKAGQQPARKRLNFG |
| | | QTGDADSVPDPQPLGQPPAAPSGLGTNTMATGSGAPMADNNEGADGVGNSSGNWHCD |
| | | STWMGDRVITTSTRTWALPTYNNHLYKQISSQSGASNDNHYFGYSTPWGYFDFNRFHC |
| | | HFSPRDWQRLINNNWGFRPKRLNFKLFNIQVKEVTQNDGTTTIANNLTSTVQVFTDSEY |
| | | QLPYVLGSAHQGCLPPFPADVFMVPQYGYLTLNNGSQAVGRSSFYCLEYFPSQMLRTGN |
| | | NFTFSYTFEDVPFHSSYAHSQSLDRLMNPLIDQYLYYLSRTNTPSGTTTQSRLQFSQAGAS |
| | | DIRDQSRNWLPGPCYRQQRVSKTSADNNNSEYSWTGATKYHLNGRDSLVNPGPAMAS |
| | | HKDDEEKFFPQSGVLIFGKQGSEKTNVDIEKVMITDEEEIRTTNPVATEQYGSVSTNLQR |
| | | GNRQAATADVNTQGVLPGMVWQDRDVYLQGPIWAKIPHTDGHFHPSPLMGGFGLKHP |
| | | PPQILIKNTPVPANPSTTFSAAKFASFITQYSTGQVSVEIEWELQKENSKRWNPEIQYTSNY |
| | | NKSVNVDFTVDTNGVYSEPRPIGTRYLTRNL* (SEQ ID NO: 7) |
| AAV2 | AAC03780.1 | ATGGCTGCCGATGGTTATCTTCCAGATTGGCTCGAGGACACTCTCTCTGAAGGAATA |
| | | AGACAGTGGTGGAAGCTCAAACCTGGCCCACCACCACCAAAGCCCGCAGAGCGGCA |
| | | TAAGGACGACAGCAGGGGTCTTGTGCTTCCTGGGTACAAGTACCTCGGACCCTTCAA |
| | | CGGACTCGACAAGGGAGAGCCGGTCAACGAGGCAGACGCCGCGGCCCTCGAGCACG |
| | | ACAAAGCCTACGACCGGCAGCTCGACAGCGGAGACAACCCGTACCTCAAGTACAAC |
| | | CACGCCGACGCGGAGTTTCAGGAGCGCCTTAAAGAAGATACGTCTTTTGGGGGCAA |
| | | CCTCGGACGAGCAGTCTTCCAGGCGAAAAAGAGGGTTCTTGAACCTCTGGGCCTGGT |
| | | TGAGGAACCTGTTAAGACGGCTCCGGGAAAAAAGAGGCCGGTAGAGCACTCTCCTG |
| | | TGGAGCCAGACTCCTCCTCGGGAACCGGAAAGGCGGGCCAGCAGCCTGCAAGAAAA |
| | | AGATTGAATTTTGGTCAGACTGGAGACGCAGACTCAGTACCTGACCCCCAGCCTCTC |
| | | GGACAGCCACCAGCAGCCCCCTCTGGTCTGGGAACTAATACGATGGCTACAGGCAG |
| | | TGGCGCACCAATGGCAGACAATAACGAGGGCGCCGACGGAGTGGGTAATTCCTCGG |
| | | GAAATTGGCATTGCGATTCCACATGGATGGGCGACAGAGTCATCACCACCAGCACCC |
| | | GAACCTGGGCCCTGCCCACCTACAACAACCACCTCTACAAACAAATTTCCAGCCAAT |
| | | CAGGAGCCTCGAACGACAATCACTACTTTGGCTACAGCACCCCTTGGGGGTATTTTG |
| | | ACTTCAACAGATTCCACTGCCACTTTTCACCACGTGACTGGCAAAGACTCATCAACA |
| | | ACAACTGGGGATTCCGACCCAAGAGACTCAACTTCAAGCTCTTTAACATTCAAGTCA |
| | | AAGAGGTCACGCAGAATGACGGTACGACGACGATTGCCAATAACCTTACCAGCACG |
| | | GTTCAGGTGTTTACTGACTCGGAGTACCAGCTCCCGTACGTCCTCGGCTCGGCGCAT |
| | | CAAGGATGCCTCCCGCCGTTCCCAGCAGACGTCTTCATGGTGCCACAGTATGGATAC |
| | | CTCACCCTGAACAACGGGAGTCAGGCAGTAGGACGCTCTTCATTTTACTGCCTGGAG |
| | | TACTTTCCTTCTCAGATGCTGCGTACCGGAAACAACTTTACCTTCAGCTACACTTTTG |
| | | AGGACGTTCCTTTCCACAGCAGCTACGCTCACAGCCAGAGTCTGGACCGTCTCATGA |
| | | ATCCTCTCATCGACCAGTACCTGTATTACTTGAGCAGAACAAACACTCCAAGTGGAA |
| | | CCACCACGCAGTCAAGGCTTCAGTTTTCTCAGGCCGGAGCGAGTGACATTCGGGACC |
| | | AGTCTAGGAACTGGCTTCCTGGACCCTGTTACCGCCAGCAGCGAGTATCAAAGACAT |
| | | CTGCGGATAACAACAACAGTGAATACTCGTGGACTGGAGCTACCAAGTACCACCTA |
| | | ATGGCAGAGACTCTCTGGTGAATCCGGGCCCGGCCATGGCAAGCCACAAGGACGAT |
| | | GAAGAAAAGTTTTTTCCTCAGAGCGGGGTTCTCATCTTTGGGAAGCAAGGCTCAGAG |
| | | AAAACAAATGTGGACATTGAAAAGGTCATGATTACAGACGAAGAGGAAATCAGGAC |
| | | AACCAATCCCGTGGCTACGGAGCAGTATGGTTCTGTATCTACCAACCTCCAGAGAGG |
| | | CAACAGACAAGCAGCTACCGCAGATGTCAACACACAAGGCGTTCTTCCAGGCATGG |
| | | TCTGGCAGGACAGAGATGTGTACCTTCAGGGGCCCATCTGGGCAAAGATTCCACACA |
| | | CGGACGGACATTTTCACCCCTCTCCCCTCATGGGTGGATTCGGACTTAAACACCCTCC |
| | | TCCACAGATTCTCATCAAGAACACCCCGGTACCTGCGAATCCTTCGACCACCTTCAG |
| | | TGCGGCAAAGTTTGCTTCCTTCATCACACAGTACTCCACGGGACAGGTCAGCGTGGA |
| | | GATCGAGTGGGAGCTGCAGAAGGAAAACAGCAAACGCTGGAATCCCGAAATTCAGT |
| | | ACACTTCCAACTACAACAAGTCTGTTAATGTGGACTTTACTGTGGACACTAATGGCG |
| | | TGTATTCAGAGCCTCGCCCCATTGGCACCAGATACCTGACTCGTAATCTGTAA (SEQ |
| | | ID NO: 8) |
| AAV3B | AAB95452.1 | MAADGYLPDWLEDNLSEGIREWWALKPGVPQPKANQQHQDNRRGLVLPGYKYLGPG |
| | | NGLDKGEPVNEADAAALEHDKAYDQQLKAGDNPYLKYNHADAEFQERLQEDTSFGGN |
| | | LGRAVFQAKKRILEPLGLVEEAAKTAPGKKRPVDQSPQEPDSSSGVGKSGKQPARKRLN |
| | | FGQTGDSESVPDPQPLGEPPAAPTSLGSNTMASGGGAPMADNNEGADGVGNSSGNWHC |
| | | DSQWLGDRVITTSTRTWALPTYNNHLYKQISSQSGASNDNHYFGYSTPWGYFDFNRFHC |
| | | HFSPRDWQRLINNNWGFRPKKLSFKLFNIQVKEVTQNDGTTTIANNLTSTVQVFTDSEQL |
| | | PYVLGSAHQGCLPPFPADVFMVPQYGYLTLNNGSQAVGRSSFYCLEYFPSQMLRTGNNF |
| | | QFSYTFEDVPFHSSYAHSQSLDRLMNPLIDQYLYYLNRTQGTTSGTTNQSRLLFSQAGPQ |
| | | SMSLQARNWLPGPCYRQQRLSKTANDNNNSNFPWTAASKYHLNGRDSLVNPGPAMAS |
| | | HKDDEEKFFPMHGNLIFGKEGTTASNAELDNVMITDEEEIRTTNPVATEQYGTVANNLQ |
| | | SSNTAPTTRTVNDQGALPGMVWQDRDVYLQGPIWAKIPHTDGHFHPSPLMGGFGLKHP |
| | | PPQIMIKNTPVPANPPTTFSPAKFASFITQYSTGQVSVEIEWELQKENSKRWNPEIQYTSN |
| | | YNKSVNVDFTVDTNGVYSEPRPIGTRYLTRNL* (SEQ ID NO: 9) |
| AAV3B | AAB95452.1 | ATGGCTGCTGACGGTTATCTTCCAGATTGGCTCGAGGACAACCTTTCTGAAGGCATT |
| | | CGTGAGTGGTGGGCTCTGAAACCTGGAGTCCCTCAACCCAAAGCGAACCAACAACA |
| | | CCAGGACAACCGTCGGGGTCTTGTGCTTCCGGGTTACAAATACCTCGGACCCGGTAA |
| | | CGGACTCGACAAAGGAGAGCCGGTCAACGAGGCGGACGCGGCAGCCCTCGAACACG |
| | | ACAAAGCTTACGACCAGCAGCTCAAGGCCGGTGACAACCCGTACCTCAAGTACAAC |
| | | CACGCCGACGCCGAGTTTCAGGAGCGTCTTCAAGAAGATACGTCTTTTGGGGGCAAC |
| | | CTTGGCAGAGCAGTCTTCCAGGCCAAAAAGAGGATCCTTGAGCCTCTTGGTCTGGTT |
| | | GAGGAAGCAGCTAAAACGGCTCCTGGAAAGAAGAGGCCTGTAGATCAGTCTCCTCA |
| | | GGAACCGGACTCATCATCTGGTGTTGGCAAATCGGGCAAACAGCCTGCCAGAAAAA |
| | | GACTAAATTTCGGTCAGACTGGCGACTCAGAGTCAGTCCCAGACCCTCAACCTCTCG |
| | | GAGAACCACCAGCAGCCCCCACAAGTTTGGGATCTAATACAATGGCTTCAGGCGGT |
| | | GGCGCACCAATGGCAGACAATAACGAGGGTGCCGATGGAGTGGGTAATTCCTCAGG |
| | | AAATTGGCATTGCGATTCCCAATGGCTGGGCGACAGAGTCATCACCACCAGCACCAG |
| | | AACCTGGGCCCTGCCCACTTACAACAACCATCTCTACAAGCAAATCTCCAGCCAATC |
| | | AGGAGCTTCAAACGACAACCACTACTTTGGCTACAGCACCCCTTGGGGGTATTTTGA |
| | | CTTTAACAGATTCCACTGCCACTTCTCACCACGTGACTGGCAGCGACTCATTAACAA |
| | | CAACTGGGGATTCCGGCCCAAGAAACTCAGCTTCAAGCTCTTCAACATCCAAGTTAA |
| | | AGAGGTCACGCAGAACGATGGCACGACGACTATTGCCAATAACCTTACCAGCACGG |
| | | TTCAAGTGTTTACGGACTCGGAGTATCAGCTCCCGTACGTGCTCGGGTCGGCGCACC |
| | | AAGGCTGTCTCCCGCCGTTTCCAGCGGACGTCTTCATGGTCCCTCAGTATGGATACCT |
| | | CACCCTGAACAACGGAAGTCAAGCGGTGGGACGCTCATCCTTTTACTGCCTGGAGTA |
| | | CTTCCCTTCGCAGATGCTAAGGACTGGAAATAACTTCCAATTCAGCTATACCTTCGA |
| | | GGATGTACCTTTTCACAGCAGCTACGCTCACAGCCAGAGTTTGGATCGCTTGATGAA |
| | | TCCTCTTATTGATCAGTATCTGTACTACCTGAACAGAACGCAAGGAACAACCTCTGG |
| | | AACAACCAACCAATCACGGCTGCTTTTTAGCCAGGCTGGGCCTCAGTCTATGTCTTT |
| | | GCAGGCCAGAAATTGGCTACCTGGGCCCTGCTACCGGCAACAGAGACTTTCAAAGA |
| | | CTGCTAACGACAACAACAACAGTAACTTTCCTTGGACAGCGGCCAGCAAATATCATC |
| | | TCAATGGCCGCGACTCGCTGGTGAATCCAGGACCAGCTATGGCCAGTCACAAGGAC |
| | | GATGAAGAAAAATTTTTCCCTATGCACGGCAATCTAATATTTGGCAAAGAAGGGACA |
| | | ACGGCAAGTAACGCAGAATTAGATAATGTAATGATTACGGATGAAGAAGAGATTCG |
| | | TACCACCAATCCTGTGGCAACAGAGCAGTATGGAACTGTGGCAAATAACTTGCAGA |
| | | GCTCAAATACAGCTCCCACGACTAGAACTGTCAATGATCAGGGGGCCTTACCTGGCA |
| | | TGGTGTGGCAAGATCGTGACGTGTACCTTCAAGGACCTATCTGGGCAAAGATTCCTC |
| | | ACACGGATGGACACTTTCATCCTTCTCCTCTGATGGGAGGCTTTGGACTGAAACATC |
| | | CGCCTCCTCAAATCATGATCAAAAATACTCCGGTACCGGCAAATCCTCCGACGACTT |
| | | TCAGCCCGGCCAAGTTTGCTTCATTTATCACTCAGTACTCCACTGGACAGGTCAGCGT |
| | | GGAAATTGAGTGGGAGCTACAGAAAGAAAACAGCAAACGTTGGAATCCAGAGATTC |
| | | AGTACACTTCCAACTACAACAAGTCTGTTAATGTGGACTTTACTGTAGACACTAATG |
| | | GTGTTTATAGTGAACCTCGCCCTATTGGAACCCGGTATCTCACACGAAACTTGTAA |
| | | (SEQ ID NO: 10) |
| AAV4 | AAC58045 | MTDGYLPDWLEDNLSEGVREWWALQPGAPKPKANQQHQDNARGLVLPGYKYLGPGN |
| | | GLDKGEPVNAADAAALEHDKAYDQQLKAGDNPYLKYNHADAEFQQRLQGDTSFGGN |
| | | LGRAVFQAKKRVLEPLGLVEQAGETAPGKKRPLIESPQQPDSSTGIGKKGKQPAKKKLV |
| | | FEDETGAGDGPPEGSTSGAMSDDSEMRAAAGGAAVEGGQGADGVGNASGDWHCDST |
| | | WSEGHVTTTSTRTWVLPTYNNHLYKRLGESLQSNTYNGFSTPWGYFDFNRFHCHFSPRD |
| | | WQRLINNNWGMRPKAMRVKIFNIQVKEVTTSNGETTVANNLTSTVQIFADSSYELPYV |
| | | MDAGQEGSLPPFPNDVFMVPQYGYCGLVTGNTSQQQTDRNAFYCLEYFPSQMLRTGNN |
| | | FEITYSFEKVPFHSMYAHSQSLDRLMNPLIDQYLWGLQSTTTGTTLNAGTATTNFTKLRP |
| | | TNFSNFKKNWLPGPSIKQQGFSKTANQNYKIPATGSDSLIKYETHSTLDGRWSALTPGPP |
| | | MATAGPADSKFSNSQLIFAGPKQNGNTATVPGTLIFTSEEELAATNATDTDMWGNLPGG |
| | | DQSNSNLPTVDRLTALGAVPGMVWQNRDIYYQGPIWAKIPHTDGHFHPSPLIGGFGLKH |
| | | PPPQIFIKNTPVPANPATTFSSTPVNSFITQYSTGQVSVQIDWEIQKERSKRWNPEVQFTSN |
| | | YGQQNSLLWAPDAAGKYTEPRAIGTRYLTHHL* (SEQ ID NO: 11) |
| AAV4 | AAC58045 | ATGACTGACGGTTACCTTCCAGATTGGCTAGAGGACAACCTCTCTGAAGGCGTTCGA |
| | | GAGTGGTGGGCGCTGCAACCTGGAGCCCCTAAACCCAAGGCAAATCAACAACATCA |
| | | GGACAACGCTCGGGGTCTTGTGCTTCCGGGTTACAAATACCTCGGACCCGGCAACGG |
| | | ACTCGACAAGGGGGAACCCGTCAACGCAGCGGACGCGGCAGCCCTCGAGCACGACA |
| | | AGGCCTACGACCAGCAGCTCAAGGCCGGTGACAACCCCTACCTCAAGTACAACCAC |
| | | GCCGACGCGGAGTTCCAGCAGCGGCTTCAGGGCGACACATCGTTTGGGGGCAACCT |
| | | CGGCAGAGCAGTCTTCCAGGCCAAAAAGAGGGTTCTTGAACCTCTTGGTCTGGTTGA |
| | | GCAAGCGGGTGAGACGGCTCCTGGAAAGAAGAGACCGTTGATTGAATCCCCCCAGC |
| | | AGCCCGACTCCTCCACGGGTATCGGCAAAAAAGGCAAGCAGCCGGCTAAAAAGAAG |
| | | CTCGTTTTCGAAGACGAAACTGGAGCAGGCGACGGACCCCCTGAGGGATCAACTTCC |
| | | GGAGCCATGTCTGATGACAGTGAGATGCGTGCAGCAGCTGGCGGAGCTGCAGTCGA |
| | | GGGCGGACAAGGTGCCGATGGAGTGGGTAATGCCTCGGGTGATTGGCATTGCGATT |
| | | CCACCTGGTCTGAGGGCCACGTCACGACCACCAGCACCAGAACCTGGGTCTTGCCCA |
| | | CCTACAACAACCACCTCTACAAGCGACTCGGAGAGAGCCTGCAGTCCAACACCTAC |
| | | AACGGATTCTCCACCCCCTGGGGATACTTTGACTTCAACCGCTTCCACTGCCACTTCT |
| | | CACCACGTGACTGGCAGCGACTCATCAACAACAACTGGGGCATGCGACCCAAAGCC |
| | | ATGCGGGTCAAAATCTTCAACATCCAGGTCAAGGAGGTCACGACGTCGAACGGCGA |
| | | GACAACGGTGGCTAATAACCTTACCAGCACGGTTCAGATCTTTGCGGACTCGTCGTA |
| | | CGAACTGCCGTACGTGATGGATGCGGGTCAAGAGGGCAGCCTGCCTCCTTTTCCCAA |
| | | CGACGTCTTTATGGTGCCCCAGTACGGCTACTGTGGACTGGTGACCGGCAACACTTC |
| | | GCAGCAACAGACTGACAGAAATGCCTTCTACTGCCTGGAGTACTTTCCTTCGCAGAT |
| | | GCTGCGGACTGGCAACAACTTTGAAATTACGTACAGTTTTGAGAAGGTGCCTTTCCA |
| | | CTCGATGTACGCGCACAGCCAGAGCCTGGACCGGCTGATGAACCCTCTCATCGACCA |
| | | GTACCTGTGGGGACTGCAATCGACCACCACCGGAACCACCCTGAATGCCGGGACTG |
| | | CCACCACCAACTTTACCAAGCTGCGGCCTACCAACTTTTCCAACTTTAAAAAGAACT |
| | | GGCTGCCCGGGCCTTCAATCAAGCAGCAGGGCTTCTCAAAGACTGCCAATCAAAACT |
| | | ACAAGATCCCTGCCACCGGGTCAGACAGTCTCATCAAATACGAGACGCACAGCACT |
| | | CTGGACGGAAGATGGAGTGCCCTGACCCCCGGACCTCCAATGGCCACGGCTGGACC |
| | | TGCGGACAGCAAGTTCAGCAACAGCCAGCTCATCTTTGCGGGGCCTAAACAGAACG |
| | | GCAACACGGCCACCGTACCCGGGACTCTGATCTTCACCTCTGAGGAGGAGCTGGCAG |
| | | CCACCAACGCCACCGATACGGACATGTGGGGCAACCTACCTGGCGGTGACCAGAGC |
| | | AACAGCAACCTGCCGACCGTGGACAGACTGACAGCCTTGGGAGCCGTGCCTGGAAT |
| | | GGTCTGGCAAAACAGAGACATTTACTACCAGGGTCCCATTTGGGCCAAGATTCCTCA |
| | | TACCGATGGACACTTTCACCCCTCACCGCTGATTGGTGGGTTTGGGCTGAAACACCC |
| | | GCCTCCTCAAATTTTTATCAAGAACACCCCGGTACCTGCGAATCCTGCAACGACCTT |
| | | CAGCTCTACTCCGGTAAACTCCTTCATTACTCAGTACAGCACTGGCCAGGTGTCGGT |
| | | GCAGATTGACTGGGAGATCCAGAAGGAGCGGTCCAAACGCTGGAACCCCGAGGTCC |
| | | AGTTTACCTCCAACTACGGACAGCAAAACTCTCTGTTGTGGGCTCCCGATGCGGCTG |
| | | GGAAATACACTGAGCCTAGGGCTATCGGTACCCGCTACCTCACCCACCACCTG (SEQ |
| | | ID NO: 12) |
| AAV5 | AAD13756.1 | MSFVDHPPDWLEEVGEGLREFLGLEAGPPKPKPNQQHQDQARGLVLPGYNYLGPGNGL |
| | | DRGEPVNRADEVAREHDISYNEQLEAGDNPYLKYNHADAEFQEKLADDTSFGGNLGKA |
| | | VFQAKKRVLEPFGLVEEGAKTAPTGKRIDDHFPKRKKARTEEDSKPSTSSDAEAGPSGSQ |
| | | QLQIPAQPASSLGADTMSAGGGGPLGDNNQGADGVGNASGDWHCDSTWMGDRVVTK |
| | | STRTWVLPSYNNHQYREIKSGSVDGSNANAYFGYSTPWGYFDFNRFHSHWSPRDWQRL |
| | | INNYWGFRPRSLRVKIFNIQVKEVTVQDSTTTIANNLTSTVQVFTDDDYQLPYVVGNGTE |
| | | GCLPAFPPQVFTLPQYGYATLNRDNTENPTERSSFFCLEYFPSKMLRTGNNFEFTYNFEE |
| | | VPFHSSFAPSQNLFKLANPLVDQYLYRFVSTNNTGGVQFNKNLAGRYANTYKNWFPGP |
| | | MGRTQGWNLGSGVNRASVSAFATTNRMELEGASYQVPPQPNGMTNNLQGSNTYALEN |
| | | TMIFNSQPANPGTTATYLEGNMLITSESETQPVNRVAYNVGGQMATNNQSSTTAPATGT |
| | | YNLQEIVPGSVWMERDVYLQGPIWAKIPETGAHFHPSPAMGGFGLKHPPPMMLIKNTPV |
| | | PGNITSFSDVPVSSFITQYSTGQVTVEMEWELKKENSKRWNPEIQYTNNYNDPQFVDFAP |
| | | DSTGEYRTTRPIGTRYLTRPL* (SEQ ID NO: 13) |
| AAV5 | AAD13756.1 | ATGTCTTTTGTTGATCACCCTCCAGATTGGTTGGAAGAAGTTGGTGAAGGTCTTCGCG |
| | | AGTTTTTGGGCCTTGAAGCGGGCCCACCGAAACCAAAACCCAATCAGCAGCATCAA |
| | | GATCAAGCCCGTGGTCTTGTGCTGCCTGGTTATAACTATCTCGGACCCGGAAACGGT |
| | | CTCGATCGAGGAGAGCCTGTCAACAGGGCAGACGAGGTCGCGCGAGAGCACGACAT |
| | | CTCGTACAACGAGCAGCTTGAGGCGGGAGACAACCCCTACCTCAAGTACAACCACG |
| | | CGGACGCCGAGTTTCAGGAGAAGCTCGCCGACGACACATCCTTCGGGGGAAACCTC |
| | | GGAAAGGCAGTCTTTCAGGCCAAGAAAAGGGTTCTCGAACCTTTTGGCCTGGTTGAA |
| | | GAGGGTGCTAAGACGGCCCCTACCGGAAAGCGGATAGACGACCACTTTCCAAAAAG |
| | | AAAGAAGGCTCGGACCGAAGAGGACTCCAAGCCTTCCACCTCGTCAGACGCCGAAG |
| | | CTGGACCCAGCGGATCCCAGCAGCTGCAAATCCCAGCCCAACCAGCCTCAAGTTTGG |
| | | GAGCTGATACAATGTCTGCGGGAGGTGGCGGCCCATTGGGCGACAATAACCAAGGT |
| | | GCCGATGGAGTGGGCAATGCCTCGGGAGATTGGCATTGCGATTCCACGTGGATGGG |
| | | GGACAGAGTCGTCACCAAGTCCACCCGAACCTGGGTGCTGCCCAGCTACAACAACC |
| | | ACCAGTACCGAGAGATCAAAAGCGGCTCCGTCGACGGAAGCAACGCCAACGCCTAC |
| | | TTTGGATACAGCACCCCCTGGGGGTACTTTGACTTTAACCGCTTCCACAGCCACTGG |
| | | AGCCCCCGAGACTGGCAAAGACTCATCAACAACTACTGGGGCTTCAGACCCCGGTCC |
| | | CTCAGAGTCAAAATCTTCAACATTCAAGTCAAAGAGGTCACGGTGCAGGACTCCACC |
| | | ACCACCATCGCCAACAACCTCACCTCCACCGTCCAAGTGTTTACGGACGACGACTAC |
| | | CAGCTGCCCTACGTCGTCGGCAACGGGACCGAGGGATGCCTGCCGGCCTTCCCTCCG |
| | | CAGGTCTTTACGCTGCCGCAGTACGGTTACGCGACGCTGAACCGCGACAACACAGA |
| | | AAATCCCACCGAGAGGAGCAGCTTCTTCTGCCTAGAGTACTTTCCCAGCAAGATGCT |
| | | GAGAACGGGCAACAACTTTGAGTTTACCTACAACTTTGAGGAGGTGCCCTTCCACTC |
| | | CAGCTTCGCTCCCAGTCAGAACCTGTTCAAGCTGGCCAACCCGCTGGTGGACCAGTA |
| | | CTTGTACCGCTTCGTGAGCACAAATAACACTGGCGGAGTCCAGTTCAACAAGAACCT |
| | | GGCCGGGAGATACGCCAACACCTACAAAAACTGGTTCCCGGGGCCCATGGGCCGAA |
| | | CCCAGGGCTGGAACCTGGGCTCCGGGGTCAACCGCGCCAGTGTCAGCGCCTTCGCCA |
| | | CGACCAATAGGATGGAGCTCGAGGGCGCGAGTTACCAGGTGCCCCCGCAGCCGAAC |
| | | GGCATGACCAACAACCTCCAGGGCAGCAACACCTATGCCCTGGAGAACACTATGAT |
| | | CTTCAACAGCCAGCCGGCGAACCCGGGCACCACCGCCACGTACCTCGAGGGCAACA |
| | | TGCTCATCACCAGCGAGAGCGAGACGCAGCCGGTGAACCGCGTGGCGTACAACGTC |
| | | GGCGGGCAGATGGCCACCAACAACCAGAGCTCCACCACTGCCCCCGCGACCGGCAC |
| | | GTACAACCTCCAGGAAATCGTGCCCGGCAGCGTGTGGATGGAGAGGGACGTGTACC |
| | | TCCAAGGACCCATCTGGGCCAAGATCCCAGAGACGGGGGCGCACTTTCACCCCTCTC |
| | | CGGCCATGGGCGGATTCGGACTCAAACACCCACCGCCCATGATGCTCATCAAGAAC |
| | | ACGCCTGTGCCCGGAAATATCACCAGCTTCTCGGACGTGCCCGTCAGCAGCTTCATC |
| | | ACCCAGTACAGCACCGGGCAGGTCACCGTGGAGATGGAGTGGGAGCTCAAGAAGGA |
| | | AAACTCCAAGAGGTGGAACCCAGAGATCCAGTACACAAACAACTACAACGACCCCC |
| | | AGTTTGTGGACTTTGCCCCGGACAGCACCGGGGAATACAGAACCACCAGACCTATCG |
| | | GAACCCGATACCTTACCCGACCCCTTTAA (SEQ ID NO: 14) |
| AAV6 | AAB95450.1 | MAADGYLPDWLEDNLSEGIREWWDLKPGAPKPKANQQKQDDGRGLVLPGYKYLGPFN |
| | | GLDKGEPVNAADAAALEHDKAYDQQLKAGDNPYLRYNHADAEFQERLQEDTSFGGNL |
| | | GRAVFQAKKRVLEPFGLVEEGAKTAPGKKRPVEQSPQEPDSSSGIGKTGQQPAKKRLNF |
| | | GQTGDSESVPDPQPLGEPPATPAAVGPTTMASGGGAPMADNNEGADGVGNASGNWHC |
| | | DSTWLGDRVITTSTRTWALPTYNNHLYKQISSASTGASNDNHYFGYSTPWGYFDFNRFH |
| | | CHFSPRDWQRLINNNWGFRPKRLNFKLFNIQVKEVTTNDGVTTIANNLTSTVQVFSDSE |
| | | YQLPYVLGSAHQGCLPPFPADVFMIPQYGYLTLNNGSQAVGRSSFYCLEYFPSQMLRTG |
| | | NNFTFSYTFEDVPFHSSYAHSQSLDRLMNPLIDQYLYYLNRTQNQSGSAQNKDLLFSRGS |
| | | PAGMSVQPKNWLPGPCYRQQRVSKTKTDNNNSNFTWTGASKYNLNGRESIINPGTAMA |
| | | SHKDDKDKFFPMSGVMIFGKESAGASNTALDNVMITDEEEIKATNPVATERFGTVAVNL |
| | | QSSSTDPATGDVHVMGALPGMVWQDRDVYLQGPIWAKIPHTDGHFHPSPLMGGFGLK |
| | | HPPPQILIKNTPVPANPPAEFSATKFASFITQYSTGQVSVEIEWELQKENSKRWNPEVQYT |
| | | SNYAKSANVDFTVDNNGLYTEPRPIGTRYLTRPL* (SEQ ID NO: 15) |
| AAV6 | AAB95450.1 | ATGGCTGCCGATGGTTATCTTCCAGATTGGCTCGAGGACAACCTCTCTGAGGGCATT |
| | | CGCGAGTGGTGGGACTTGAAACCTGGAGCCCCGAAACCCAAAGCCAACCAGCAAAA |
| | | GCAGGACGACGGCCGGGGTCTGGTGCTTCCTGGCTACAAGTACCTCGGACCCTTCAA |
| | | CGGACTCGACAAGGGGGAGCCCGTCAACGCGGCGGATGCAGCGGCCCTCGAGCACG |
| | | ACAAGGCCTACGACCAGCAGCTCAAAGCGGGTGACAATCCGTACCTGCGGTATAAC |
| | | CACGCCGACGCCGAGTTTCAGGAGCGTCTGCAAGAAGATACGTCTTTTGGGGGCAAC |
| | | CTCGGGCGAGCAGTCTTCCAGGCCAAGAAGAGGGTTCTCGAACCTTTTGGTCTGGTT |
| | | GAGGAAGGTGCTAAGACGGCTCCTGGAAAGAAACGTCCGGTAGAGCAGTCGCCACA |
| | | AGAGCCAGACTCCTCCTCGGGCATTGGCAAGACAGGCCAGCAGCCCGCTAAAAAGA |
| | | GACTCAATTTTGGTCAGACTGGCGACTCAGAGTCAGTCCCCGACCCACAACCTCTCG |
| | | GAGAACCTCCAGCAACCCCCGCTGCTGTGGGACCTACTACAATGGCTTCAGGCGGTG |
| | | GCGCACCAATGGCAGACAATAACGAAGGCGCCGACGGAGTGGGTAATGCCTCAGGA |
| | | AATTGGCATTGCGATTCCACATGGCTGGGCGACAGAGTCATCACCACCAGCACCCGA |
| | | ACATGGGCCTTGCCCACCTATAACAACCACCTCTACAAGCAAATCTCCAGTGCTTCA |
| | | ACGGGGGCCAGCAACGACAACCACTACTTCGGCTACAGCACCCCCTGGGGGTATTTT |
| | | GATTTCAACAGATTCCACTGCCATTTCTCACCACGTGACTGGCAGCGACTCATCAAC |
| | | AACAATTGGGGATTCCGGCCCAAGAGACTCAACTTCAAGCTCTTCAACATCCAAGTC |
| | | AAGGAGGTCACGACGAATGATGGCGTCACGACCATCGCTAATAACCTTACCAGCAC |
| | | GGTTCAAGTCTTCTCGGACTCGGAGTACCAGTTGCCGTACGTCCTCGGCTCTGCGCA |
| | | CCAGGGCTGCCTCCCTCCGTTCCCGGCGGACGTGTTCATGATTCCGCAGTACGGCTA |
| | | CCTAACGCTCAACAATGGCAGCCAGGCAGTGGGACGGTCATCCTTTTACTGCCTGGA |
| | | ATATTTCCCATCGCAGATGCTGAGAACGGGCAATAACTTTACCTTCAGCTACACCTC |
| | | GAGGACGTGCCTTTCCACAGCAGCTACGCGCACAGCCAGAGCCTGGACCGGCTGAT |
| | | GAATCCTCTCATCGACCAGTACCTGTATTACCTGAACAGAACTCAGAATCAGTCCGG |
| | | AAGTGCCCAAAACAAGGACTTGCTGTTTAGCCGGGGGTCTCCAGCTGGCATGTCTGT |
| | | TCAGCCCAAAAACTGGCTACCTGGACCCTGTTACCGGCAGCAGCGCGTTTCTAAAAC |
| | | AAAAACAGACAACAACAACAGCAACTTTACCTGGACTGGTGCTTCAAAATATAACC |
| | | TTAATGGGCGTGAATCTATAATCAACCCTGGCACTGCTATGGCCTCACACAAAGACG |
| | | ACAAAGACAAGTTCTTTCCCATGAGCGGTGTCATGATTTTTGGAAAGGAGAGCGCCG |
| | | GAGCTTCAAACACTGCATTGGACAATGTCATGATCACAGACGAAGAGGAAATCAAA |
| | | GCCACTAACCCCGTGGCCACCGAAAGATTTGGGACTGTGGCAGTCAATCTCCAGAGC |
| | | AGCAGCACAGACCCTGCGACCGGAGATGTGCATGTTATGGGAGCCTTACCTGGAAT |
| | | GGTGTGGCAAGACAGAGACGTATACCTGCAGGGTCCTATTTGGGCCAAAATTCCTCA |
| | | CACGGATGGACACTTTCACCCGTCTCCTCTCATGGGCGGCTTTGGACTTAAGCACCC |
| | | GCCTCCTCAGATCCTCATCAAAAACACGCCTGTTCCTGCGAATCCTCCGGCAGAGTT |
| | | TTCGGCTACAAAGTTTGCTTCATTCATCACCCAGTATTCCACAGGACAAGTGAGCGT |
| | | GGAGATTGAATGGGAGCTGCAGAAAGAAAACAGCAAACGCTGGAATCCCGAAGTGC |
| | | AGTATACATCTAACTATGCAAAATCTGCCAACGTTGATTTCACTGTGGACAACAATG |
| | | GACTTTATACTGAGCCTCGCCCCATTGGCACCCGTTACCTCACCCGTCCCCTGTAA |
| | | (SEQ ID NO: 16) |
| AAV7 | AAN03855.1 | MAADGYLPDWLEDNLSEGIREWWDLKPGAPKPKANQQKQDNGRGLVLPGYKYLGPFN |
| | | GLDKGEPVNAADAAALEHDKAYDQQLKAGDNPYLRYNHADAEFQERLQEDTSFGGNL |
| | | GRAVFQAKKRVLEPLGLVEEGAKTAPAKKRPVEPSPQRSPDSSTGIGKKGQQPARKRLN |
| | | FGQTGDSESVPDPQPLGEPPAAPSSVGSGTVAAGGGAPMADNNEGADGVGNASGNWH |
| | | CDSTWLGDRVITTSTRTWALPTYNNHLYKQISSETAGSTNDNTYFGYSTPWGYFDFNRF |
| | | HCHFSPRDWQRLINNNWGFRPKKLRFKLFNIQVKEVTTNDGVTTIANNLTSTIQVFSDSE |
| | | YQLPYVLGSAHQGCLPPFPADVFMIPQYGYLTLNNGSQSVGRSSFYCLEYFPSQMLRTG |
| | | NNFEFSYSFEDVPFHSSYAHSQSLDRLMNPLIDQYLYYLARTQSNPGGTAGNRELQFYQ |
| | | GGPSTMAEQAKNWLPGPCFRQQRVSKTLDQNNNSNFAWTGATKYHLNGRNSLVNPGV |
| | | AMATHKDDEDRFFPSSGVLIFGKTGATNKTTLENVLMTNEEEIRPTNPVATEEYGIVSSN |
| | | LQAANTAAQTQVVNNQGALPGMVWQNRDVYLQGPIWAKIPHTDGNFHPSPLMGGFGL |
| | | KHPPPQILIKNTPVPANPPEVFTPAKFASFITQYSTGQVSVEIEWELQKENSKRWNPEIQYT |
| | | SNFEKQTGVDFAVDSQGVYSEPRPIGTRYLTRNL* (SEQ ID NO: 17) |
| AAV7 | AAN03855.1 | ATGGCTGCCGATGGTTATCTTCCAGATTGGCTCGAGGACAACCTCTCTGAGGGCATT |
| | | CGCGAGTGGTGGGACCTGAAACCTGGAGCCCCGAAACCCAAAGCCAACCAGCAAAA |
| | | GCAGGACAACGGCCGGGGTCTGGTGCTTCCTGGCTACAAGTACCTCGGACCCTTCAA |
| | | CGGACTCGACAAGGGGGAGCCCGTCAACGCGGCGGACGCAGCGGCCCTCGAGCACG |
| | | ACAAGGCCTACGACCAGCAGCTCAAAGCGGGTGACAATCCGTACCTGCGGTATAAC |
| | | CACGCCGACGCCGAGTTTCAGGAGCGTCTGCAAGAAGATACGTCATTTGGGGGCAA |
| | | CCTCGGGCGAGCAGTCTTCCAGGCCAAGAAGCGGGTTCTCGAACCTCTCGGTCTGGT |
| | | TGAGGAAGGCGCTAAGACGGCTCCTGCAAAGAAGAGACCGGTAGAGCCGTCACCTC |
| | | AGCGTTCCCCCGACTCCTCCACGGGCATCGGCAAGAAAGGCCAGCAGCCCGCCAGA |
| | | AAGAGACTCAATTTCGGTCAGACTGGCGACTCAGAGTCAGTCCCCGACCCTCAACCT |
| | | CTCGGAGAACCTCCAGCAGCGCCCTCTAGTGTGGGATCTGGTACAGTGGCTGCAGGC |
| | | GGTGGCGCACCAATGGCAGACAATAACGAAGGTGCCGACGGAGTGGGTAATGCCTC |
| | | AGGAAATTGGCATTGCGATTCCACATGGCTGGGCGACAGAGTCATTACCACCAGCAC |
| | | CCGAACCTGGGCCCTGCCCACCTACAACAACCACCTCTACAAGCAAATCTCCAGTGA |
| | | AACTGCAGGTAGTACCAACGACAACACCTACTTCGGCTACAGCACCCCCTGGGGGTA |
| | | TTTTGACTTTAACAGATTCCACTGCCACTTCTCACCACGTGACTGGCAGCGACTCATC |
| | | AACAACAACTGGGGATTCCGGCCCAAGAAGCTGCGGTTCAAGCTCTTCAACATCCAG |
| | | GTCAAGGAGGTCACGACGAATGACGGCGTTACGACCATCGCTAATAACCTTACCAG |
| | | CACGATTCAGGTATTCTCGGACTCGGAATACCAGCTGCCGTACGTCCTCGGCTCTGC |
| | | GCACCAGGGCTGCCTGCCTCCGTTCCCGGCGGACGTCTTCATGATTCCTCAGTACGG |
| | | CTACCTGACTCTCAACAATGGCAGTCAGTCTGTGGGACGTTCCTCCTTCTACTGCCTG |
| | | GAGTACTTCCCCTCTCAGATGCTGAGAACGGGCAACAACTTTGAGTTCAGCTACAGC |
| | | TTCGAGGACGTGCCTTTCCACAGCAGCTACGCACACAGCCAGAGCCTGGACCGGCTG |
| | | ATGAATCCCCTCATCGACCAGTACTTGTACTACCTGGCCAGAACACAGAGTAACCCA |
| | | GGAGGCACAGCTGGCAATCGGGAACTGCAGTTTTACCAGGGCGGGCCTTCAACTAT |
| | | GGCCGAACAAGCCAAGAATTGGTTACCTGGACCTTGCTTCCGGCAACAAAGAGTCTC |
| | | CAAAACGCTGGATCAAAACAACAACAGCAACTTTGCTTGGACTGGTGCCACCAAAA |
| | | TCACCTGAACGGCAGAAACTCGTTGGTTAATCCCGGCGTCGCCATGGCAACTCACAA |
| | | GGACGACGAGGACCGCTTTTTCCCATCCAGCGGAGTCCTGATTTTTGGAAAAACTGG |
| | | AGCAACTAACAAAACTACATTGGAAAATGTGTTAATGACAAATGAAGAAGAAATTC |
| | | GTCCTACTAATCCTGTAGCCACGGAAGAATACGGGATAGTCAGCAGCAACTTACAA |
| | | GCGGCTAATACTGCAGCCCAGACACAAGTTGTCAACAACCAGGGAGCCTTACCTGG |
| | | CATGGTCTGGCAGAACCGGGACGTGTACCTGCAGGGTCCCATCTGGGCCAAGATTCC |
| | | TCACACGGATGGCAACTTTCACCCGTCTCCTTTGATGGGCGGCTTTGGACTTAAACAT |
| | | CCGCCTCCTCAGATCCTGATCAAGAACACTCCCGTTCCCGCTAATCCTCCGGAGGTG |
| | | TTTACTCCTGCCAAGTTTGCTTCGTTCATCACACAGTACAGCACCGGACAAGTCAGC |
| | | GTGGAAATCGAGTGGGAGCTGCAGAAGGAAAACAGCAAGCGCTGGAACCCGGAGA |
| | | TTCAGTACACCTCCAACTTTGAAAAGCAGACTGGTGTGGACTTTGCCGTTGACAGCC |
| | | AGGGTGTTTACTCTGAGCCTCGCCCTATTGGCACTCGTTACCTCACCCGTAATCTGTA |
| | | A (SEQ ID NO: 18) |
| AAV8 | AAN03857.1 | MAADGYLPDWLEDNLSEGIREWWALKPGAPKPKANQQKQDDGRGLVLPGYKYLGPFN |
| | | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKRLN |
| | | FGQTGDSESVPDPQPLGEPPAAPSGVGPNTMAAGGGAPMADNNEGADGVGSSSGNWH |
| | | CDSTWLGDRVITTSTRTWALPTYNNHLYKQISNGTSGGATNDNTYFGYSTPWGYFDFN |
| | | RFHCHFSPRDWQRLINNNWGFRPKRLSFKLFNIQVKEVTQNEGTKTIANNLTSTIQVFTD |
| | | SEYQLPYVLGSAHQGCLPPFPADVFMIPQYGYLTLNNGSQAVGRSSFYCLEYFPSQMLR |
| | | TGNNFQFTYTFEDVPFHSSYAHSQSLDRLMNPLIDQYLYYLSRTQTTGGTANTQTLGFSQ |
| | | GGPNTMANQAKNWLPGPCYRQQRVSTTTGQNNNSNFAWTAGTKYHLNGRNSLANPGI |
| | | AMATHKDDEERFFPSNGILIFGKQNAARDNADYSDVMLTSEEEIKTTNPVATEEYGIVAD |
| | | NLQQQNTAPQIGTVNSQGALPGMVWQNRDVYLQGPIWAKIPHTDGNFHPSPLMGGFGL |
| | | KHPPPQILIKNTPVPADPPTTFNQSKLNSFITQYSTGQVSVEIEWELQKENSKRWNPEIQY |
| | | TSNYYKSTSVDFAVNTEGVYSEPRPIGTRYLTRNL* (SEQ ID NO: 19) |
| AAV8 | AAN03857.1 | ATGGCTGCCGATGGTTATCTTCCAGATTGGCTCGAGGACAACCTCTCTGAGGGCATT |
| | | CGCGAGTGGTGGGCGCTGAAACCTGGAGCCCCGAAGCCCAAAGCCAACCAGCAAAA |
| | | GCAGGACGACGGCCGGGGTCTGGTGCTTCCTGGCTACAAGTACCTCGGACCCTTCAA |
| | | CGGACTCGACAAGGGGGAGCCCGTCAACGCGGCGGACGCAGCGGCCCTCGAGCACG |
| | | ACAAGGCCTACGACCAGCAGCTGCAGGCGGGTGACAATCCGTACCTGCGGTATAAC |
| | | ACGCCGACGCCGAGTTTCAGGAGCGTCTGCAAGAAGATACGTCTTTTGGGGGCAACC |
| | | TCGGGCGAGCAGTCTTCCAGGCCAAGAAGCGGGTTCTCGAACCTCTCGGTCTGGTTG |
| | | AGGAAGGCGCTAAGACGGCTCCTGGAAAGAAGAGACCGGTAGAGCCATCACCCCAG |
| | | CGTTCTCCAGACTCCTCTACGGGCATCGGCAAGAAAGGCCAACAGCCCGCCAGAAA |
| | | AAGACTCAATTTTGGTCAGACTGGCGACTCAGAGTCAGTTCCAGACCCTCAACCTCT |
| | | CGGAGAACCTCCAGCAGCGCCCTCTGGTGTGGGACCTAATACAATGGCTGCAGGCG |
| | | GTGGCGCACCAATGGCAGACAATAACGAAGGCGCCGACGGAGTGGGTAGTTCCTCG |
| | | GGAAATTGGCATTGCGATTCCACATGGCTGGGCGACAGAGTCATCACCACCAGCACC |
| | | CGAACCTGGGCCCTGCCCACCTACAACAACCACCTCTACAAGCAAATCTCCAACGGG |
| | | ACATCGGGAGGAGCCACCAACGACAACACCTACTTCGGCTACAGCACCCCCTGGGG |
| | | GTATTTTGACTTTAACAGATTCCACTGCCACTTTTCACCACGTGACTGGCAGCGACTC |
| | | ATCAACAACAACTGGGGATTCCGGCCCAAGAGACTCAGCTTCAAGCTCTTCAACATC |
| | | CAGGTCAAGGAGGTCACGCAGAATGAAGGCACCAAGACCATCGCCAATAACCTCAC |
| | | CAGCACCATCCAGGTGTTTACGGACTCGGAGTACCAGCTGCCGTACGTTCTCGGCTC |
| | | TGCCCACCAGGGCTGCCTGCCTCCGTTCCCGGCGGACGTGTTCATGATTCCCCAGTA |
| | | CGGCTACCTAACACTCAACAACGGTAGTCAGGCCGTGGGACGCTCCTCCTTCTACTG |
| | | CCTGGAATACTTTCCTTCGCAGATGCTGAGAACCGGCAACAACTTCCAGTTTACTTA |
| | | CACCTTCGAGGACGTGCCTTTCCACAGCAGCTACGCCCACAGCCAGAGCTTGGACCG |
| | | GCTGATGAATCCTCTGATTGACCAGTACCTGTACTACTTGTCTCGGACTCAAACAAC |
| | | AGGAGGCACGGCAAATACGCAGACTCTGGGCTTCAGCCAAGGTGGGCCTAATACAA |
| | | TGGCCAATCAGGCAAAGAACTGGCTGCCAGGACCCTGTTACCGCCAACAACGCGTCT |
| | | CAACGACAACCGGGCAAAACAACAATAGCAACTTTGCCTGGACTGCTGGGACCAAA |
| | | TACCATCTGAATGGAAGAAATTCATTGGCTAATCCTGGCATCGCTATGGCAACACAC |
| | | AAAGACGACGAGGAGCGTTTTTTTCCCAGTAACGGGATCCTGATTTTTGGCAAACAA |
| | | AATGCTGCCAGAGACAATGCGGATTACAGCGATGTCATGCTCACCAGCGAGGAAGA |
| | | AATCAAAACCACTAACCCTGTGGCTACAGAGGAATACGGTATCGTGGCAGATAACTT |
| | | GCAGCAGCAAAACACGGCTCCTCAAATTGGAACTGTCAACAGCCAGGGGGCCTTAC |
| | | CCGGTATGGTCTGGCAGAACCGGGACGTGTACCTGCAGGGTCCCATCTGGGCCAAG |
| | | ATTCCTCACACGGACGGCAACTTCCACCCGTCTCCGCTGATGGGCGGCTTTGGCCTG |
| | | AAACATCCTCCGCCTCAGATCCTGATCAAGAACACGCCTGTACCTGCGGATCCTCCG |
| | | ACCACCTTCAACCAGTCAAAGCTGAACTCTTTCATCACGCAATACAGCACCGGACAG |
| | | GTCAGCGTGGAAATTGAATGGGAGCTGCAGAAGGAAAACAGCAAGCGCTGGAACCC |
| | | CGAGATCCAGTACACCTCCAACTACTACAAATCTACAAGTGTGGACTTTGCTGTTAA |
| | | TACAGAAGGCGTGTACTCTGAACCCCGCCCCATTGGCACCCGTTACCTCACCCGTAA |
| | | TCTGTAA (SEQ ID NO: 20) |
| AAVrh10 | AAO88201.1 | MAADGYLPDWLEDNLSEGIREWWDLKPGAPKPKANQQKQDDGRGLVLPGYKYLGPFN |
| | | GLDKGEPVNAADAAALEHDKAYDQQLKAGDNPYLRYNHADAEFQERLQEDTSFGGNL |
| | | GRAVFQAKKRVLEPLGLVEEGAKTAPGKKRPVEPSPQRSPDSSTGIGKKGQQPAKKRLN |
| | | FGQTGDSESVPDPQPIGEPPAGPSGLGSGTMAAGGGAPMADNNEGADGVGSSSGNWHC |
| | | DSTWLGDRVITTSTRTWALPTYNNHLYKQISNGTSGGSTNDNTYFGYSTPWGYFDFNRF |
| | | HCHFSPRDWQRLINNNWGFRPKRLNFKLFNIQVKEVTQNEGTKTIANNLTSTIQVFTDSE |
| | | YQLPYVLGSAHQGCLPPFPADVFMIPQYGYLTLNNGSQAVGRSSFYCLEYFPSQMLRTG |
| | | NNFEFSYQFEDVPFHSSYAHSQSLDRLMNPLIDQYLYYLSRTQSTGGTAGTQQLLFSQAG |
| | | PNNMSAQAKNWLPGPCYRQQRVSTTLSQNNNSNFAWTGATKYHLNGRDSLVNPGVM |
| | | ATHKDDEERFFPSSGVLMFGKQGAGKDNVDYSSVMLTSEEEIKTTNPVATEQYGVVAD |
| | | NLQQQNAAPIVGAVNSQGALPGMVWQNRDVYLQGPIWAKIPHTDGNFHPSPLMGGFGL |
| | | KHPPPQILIKNTPVPADPPTTFSQAKLASFITQYSTGQVSVEIEWELQKENSKRWNPEIQY |
| | | TSNYYKSTNVDFAVNTDGTYSEPRPIGTRYLTRNL* (SEQ ID NO: 21) |
| AAVrh10 | AAO88201.1 | ATGGCTGCCGATGGTTATCTTCCAGATTGGCTCGAGGACAACCTCTCTGAGGGCATT |
| | | CGCGAGTGGTGGGACTTGAAACCTGGAGCCCCGAAACCCAAAGCCAACCAGCAAAA |
| | | GCAGGACGACGGCCGGGGTCTGGTGCTTCCTGGCTACAAGTACCTCGGACCCTTCAA |
| | | CGGACTCGACAAGGGGGAGCCCGTCAACGCGGCGGACGCAGCGGCCCTCGAGCACG |
| | | ACAAGGCCTACGACCAGCAGCTCAAAGCGGGTGACAATCCGTACCTGCGGTATAAC |
| | | CACGCCGACGCCGAGTTTCAGGAGCGTCTGCAAGAAGATACGTCTTTTGGGGGCAAC |
| | | CTCGGGCGAGCAGTCTTCCAGGCCAAGAAGCGGGTTCTCGAACCTCTCGGTCTGGTT |
| | | GAGGAAGGCGCTAAGACGGCTCCTGGAAAGAAGAGACCGGTAGAGCCATCACCCCA |
| | | GCGTTCTCCAGACTCCTCTACGGGCATCGGCAAGAAAGGCCAGCAGCCCGCGAAAA |
| | | AGAGACTCAACTTTGGGCAGACTGGCGACTCAGAGTCAGTGCCCGACCCTCAACCA |
| | | ATCGGAGAACCCCCCGCAGGCCCCTCTGGTCTGGGATCTGGTACAATGGCTGCAGGC |
| | | GGTGGCGCTCCAATGGCAGACAATAACGAAGGCGCCGACGGAGTGGGTAGTTCCTC |
| | | AGGAAATTGGCATTGCGATTCCACATGGCTGGGCGACAGAGTCATCACCACCAGCA |
| | | CCCGAACCTGGGCCCTCCCCACCTACAACAACCACCTCTACAAGCAAATCTCCAACG |
| | | GGACTTCGGGAGGAAGCACCAACGACAACACCTACTTCGGCTACAGCACCCCCTGG |
| | | GGGTATTTTGACTTTAACAGATTCCACTGCCACTTCTCACCACGTGACTGGCAGCGA |
| | | CTCATCAACAACAACTGGGGATTCCGGCCCAAGAGACTCAACTTCAAGCTCTTCAAC |
| | | ATCCAGGTCAAGGAGGTCACGCAGAATGAAGGCACCAAGACCATCGCCAATAACCT |
| | | TACCAGCACGATTCAGGTCTTTACGGACTCGGAATACCAGCTCCCGTACGTCCTCGG |
| | | CTCTGCGCACCAGGGCTGCCTGCCTCCGTTCCCGGCGGACGTCTTCATGATTCCTCAG |
| | | TACGGGTACCTGACTCTGAACAATGGCAGTCAGGCCGTGGGCCGTTCCTCCTTCTAC |
| | | TGCCTGGAGTACTTTCCTTCTCAAATGCTGAGAACGGGCAACAACTTTGAGTTCAGC |
| | | TACCAGTTTGAGGACGTGCCTTTTCACAGCAGCTACGCGCACAGCCAAAGCCTGGAC |
| | | CGGCTGATGAACCCCCTCATCGACCAGTACCTGTACTACCTGTCTCGGACTCAGTCC |
| | | ACGGGAGGTACCGCAGGAACTCAGCAGTTGCTATTTTCTCAGGCCGGGCCTAATAAC |
| | | ATGTCGGCTCAGGCCAAAAACTGGCTACCCGGGCCCTGCTACCGGCAGCAACGCGTC |
| | | TCCACGACACTGTCGCAAAATAACAACAGCAACTTTGCCTGGACCGGTGCCACCAAG |
| | | TATCATCTGAATGGCAGAGACTCTCTGGTAAATCCCGGTGTCGCTATGGCAACCCAC |
| | | AAGGACGACGAAGAGCGATTTTTTCCGTCCAGCGGAGTCTTAATGTTTGGGAAACAG |
| | | GGAGCTGGAAAAGACAACGTGGACTATAGCAGCGTTATGCTAACCAGTGAGGAAGA |
| | | AATTAAAACCACCAACCCAGTGGCCACAGAACAGTACGGCGTGGTGGCCGATAACC |
| | | TGCAACAGCAAAACGCCGCTCCTATTGTAGGGGCCGTCAACAGTCAAGGAGCCTTAC |
| | | CTGGCATGGTCTGGCAGAACCGGGACGTGTACCTGCAGGGTCCTATCTGGGCCAAGA |
| | | TTCCTCACACGGACGGAAACTTTCATCCCTCGCCGCTGATGGGAGGCTTTGGACTGA |
| | | AACACCCGCCTCCTCAGATCCTGATTAAGAATACACCTGTTCCCGCGGATCCTCCAA |
| | | CTACCTTCAGTCAAGCTAAGCTGGCGTCGTTCATCACGCAGTACAGCACCGGACAGG |
| | | TCAGCGTGGAAATTGAATGGGAGCTGCAGAAAGAAAACAGCAAACGCTGGAACCCA |
| | | GAGATTCAATACACTTCCAACTACTACAAATCTACAAATGTGGACTTTGCTGTTAAC |
| | | ACAGATGGCACTTATTCTGAGCCTCGCCCCATCGGCACCCGTTACCTCACCCGTAAT |
| | | CTGTAA (SEQ ID NO: 22) |
| AAVrh8 | AAO88183.1 | MAADGYLPDWLEDNLSEGIREWWDLKPGAPKPKANQQKQDDGRGLVLPGYKYLGPFN |
| | | GLDKGEPVNAADAAALEHDKAYDQQLKAGDNPYLRYNHADAEFQERLQEDTSFGGNL |
| | | GRAVFQAKKRVLEPLGLVEEGAKTAPGKKRPVEQSPQEPDSSSGIGKTGQQPAKKRLNF |
| | | GQTGDSESVPDPQPLGEPPAAPSGLGPNTMASGGGAPMADNNEGADGVGNSSGNWHC |
| | | DSTWLGDRVITTSTRTWALPTYNNHLYKQISNGTSGGSTNDNTYFGYSTPWGYFDFNRF |
| | | HCHFSPRDWQRLINNNWGFRPKRLNFKLFNIQVKEVTTNEGTKTIANNLTSTVQVFTDSE |
| | | YQLPYVLGSAHQGCLPPFPADVFMVPQYGYLTLNNGSQALGRSSFYCLEYFPSQMLRTG |
| | | NNFQFSYTFEDVPFHSSYAHSQSLDRLMNPLIDQYLYYLVRTQTTGTGGTQTLAFSQAGP |
| | | SSMANQARNWVPGPCYRQQRVSTTTNQNNNSNFAWTGAAKFKLNGRDSLMNPGVAM |
| | | ASHKDDDDRFFPSSGVLIFGKQGAGNDGVDYSQVLITDEEEIKATNPVATEEYGAVAINN |
| | | QAANTQAQTGLVHNQGVIPGMVWQNRDVYLQGPIWAKIPHTDGNFHPSPLMGGFGLK |
| | | HPPPQILIKNTPVPADPPLTFNQAKLNSFITQYSTGQVSVEIEWELQKENSKRWNPEIQYT |
| | | SNYYKSTNVDFAVNTEGVYSEPRPIGTRYLTRNL* (SEQ ID NO: 23) |
| AAVrh8 | AAO88183.1 | ATGGCTGCCGATGGTTATCTTCCAGATTGGCTCGAGGACAACCTCTCTGAGGGCATT |
| | | CGCGAGTGGTGGGACTTGAAACCTGGAGCCCCGAAACCCAAAGCCAACCAGCAAAA |
| | | GCAGGACGACGGCCGGGGTCTGGTGCTTCCTGGCTACAAGTACCTCGGACCCTTCAA |
| | | CGGACTCGACAAGGGGGAGCCCGTCAACGCGGCGGACGCAGCGGCCCTCGAGCACG |
| | | ACAAAGCCTACGACCAGCAGCTCAAAGCGGGTGACAATCCGTACCTGCGGTATAAT |
| | | CACGCCGACGCCGAGTTTCAGGAGCGTCTGCAAGAAGATACGTCTTTTGGGGGCAAC |
| | | CTCGGGCGAGCAGTCTTCCAGGCCAAGAAGCGGGTTCTCGAACCTCTCGGTCTGGTT |
| | | GAGGAAGGCGCTAAGACGGCTCCTGGAAAGAAGAGACCGGTAGAGCAGTCGCCAC |
| | | AAGAGCCAGACTCCTCCTCGGGCATCGGCAAGACAGGCCAGCAGCCCGCTAAAAAG |
| | | AGACTCAATTTTGGTCAGACTGGCGACTCAGAGTCAGTCCCCGACCCACAACCTCTC |
| | | GGAGAACCTCCAGCAGCCCCCTCAGGTCTGGGACCTAATACAATGGCTTCAGGCGGT |
| | | GGCGCTCCAATGGCAGACAATAACGAAGGCGCCGACGGAGTGGGTAATTCCTCGGG |
| | | AAATTGGCATTGCGATTCCACATGGCTGGGGGACAGAGTCATCACCACCAGCACCCG |
| | | AACCTGGGCCCTGCCCACCTACAACAACCACCTCTACAAGCAAATCTCCAACGGCAC |
| | | CTCGGGAGGAAGCACCAACGACAACACCTATTTTGGCTACAGCACCCCCTGGGGGT |
| | | ATTTTGACTTCAACAGATTCCACTGTCACTTTTCACCACGTGACTGGCAACGACTCAT |
| | | CAACAACAATTGGGGATTCCGGCCCAAAAGACTCAACTTCAAGCTGTTCAACATCCA |
| | | GGTCAAGGAAGTCACGACGAACGAAGGCACCAAGACCATCGCCAATAATCTCACCA |
| | | GCACCGTGCAGGTCTTTACGGACTCGGAGTACCAGTTACCGTACGTGCTAGGATCCG |
| | | CTCACCAGGGATGTCTGCCTCCGTTCCCGGCGGACGTCTTCATGGTTCCTCAGTACGG |
| | | CTATTTAACTTTAAACAATGGAAGCCAAGCCCTGGGACGTTCCTCCTTCTACTGTCT |
| | | GGAGTATTTCCCATCGCAGATGCTGAGAACCGGCAACAACTTTCAGTTCAGCTACAC |
| | | CTTCGAGGACGTGCCTTTCCACAGCAGCTACGCGCACAGCCAGAGCCTGGACAGGCT |
| | | GATGAATCCCCTCATCGACCAGTACCTGTACTACCTGGTCAGAACGCAAACGACTGG |
| | | AACTGGAGGGACGCAGACTCTGGCATTCAGCCAAGCGGGTCCTAGCTCAATGGCCA |
| | | ACCAGGCTAGAAATTGGGTGCCCGGACCTTGCTACCGGCAGCAGCGCGTCTCCACGA |
| | | CAACCAACCAGAACAACAACAGCAACTTTGCCTGGACGGGAGCTGCCAAGTTTAAG |
| | | CTGAACGGCCGAGACTCTCTAATGAATCCGGGCGTGGCAATGGCTTCCCACAAGGAT |
| | | GACGACGACCGCTTCTTCCCTTCGAGCGGGGTCCTGATTTTTGGCAAGCAAGGAGCC |
| | | GGGAACGATGGAGTGGATTACAGCCAAGTGCTGATTACAGATGAGGAAGAAATCAA |
| | | GGCTACCAACCCCGTGGCCACAGAAGAATATGGAGCAGTGGCCATCAACAACCAGG |
| | | CCGCCAATACGCAGGCGCAGACCGGACTCGTGCACAACCAGGGGGTGATTCCCGGC |
| | | ATGGTGTGGCAGAATAGAGACGTGTACCTGCAGGGTCCCATCTGGGCCAAAATTCCT |
| | | CACACGGACGGCAACTTTCACCCGTCTCCCCTGATGGGCGGCTTTGGACTGAAGCAC |
| | | CCGCCTCCTCAAATTCTCATCAAGAACACACCGGTTCCAGCGGACCCGCCGCTTACC |
| | | TTCAACCAGGCCAAGCTGAACTCTTTCATCACGCAGTACAGCACCGGACAGGTCAGC |
| | | GTGGAAATCGAGTGGGAGCTGCAGAAAGAAAACAGCAAACGCTGGAATCCAGAGA |
| | | TTCAATACACTTCCAACTACTACAAATCTACAAATGTGGACTTTGCTGTCAACACGG |
| | | AGGGGGTTTATAGCGAGCCTCGCCCCATTGGCACCCGTTACCTCACCCGCAACCTGT |
| | | AA (SEQ ID NO: 24) |

### Serotypes and Design Considerations for Thermal Stability

Stability of a viral capsid protein can include the thermal stability of the viral capsid protein. Thermal stability can be accessed via melting temperature (Tₘ). This metric is the temperature at which 50% of the viral capsid protein has unfolded, with an increase in melting temperature corresponding to an increase in viral capsid stability. A thermal shift (ΔTₘ) between a wild-type or reference viral capsid protein and a variant viral capsid protein can be used to identify increased thermal stability of an engineered AAV capsid. Exemplary approaches to measure thermal stability can use Differential Scanning Fluorimetry. Thermal stability of a viral capsid protein is critical to evolvability and may impact storage and processing. In general, the native structure loses stability as potentially beneficial mutations are introduced. For example, peptides may be inserted into the viral capsid protein to give the AAV unique properties, such as expanded, altered, or narrowed tropism. However, the insertion of the peptide may result in reduced thermal stability of the capsid. In example embodiments, the residues of viral capsid proteins are modified, including making amino acid substitutions at specified positions and described in further detail herein, resulting in increased thermal stability, even when further modified with small peptide insertions. In examples described herein, the thermal stability of modified viral capsid proteins increased by 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C. or more relative to the parental serotype or naturally-occurring capsid.

The increased thermal stability of the engineered viral capsid protein maintains or increases transduction efficiency. In one embodiment, a capsid incorporating the engineered viral capsid protein has an increased transduction of 1.1, 1.2, 1.3, 1.4, 1.5, 1.6, 1.7, 1.8, 1.9, 2.0-fold or more relative to the wild-type capsid. Methods for measuring transduction efficiency are known in the art. See e.g., Lang J F et al. Standard screening methods underreport AAV-mediated transduction and gene editing. Nat Commun. 2019 Jul. 30; 10(1):3415. Without being bound by theory, the one or more mutations described herein may increase transduction by improving the efficiency of one or more aspects of the transduction process, for example, binding, transcytosis, endosomal escape, nuclear translocation, or uncoating. Engineered variants can maintain VP1:VP2:VP3 stoichiometry, capsid assembly and cargo packaging. In embodiments, engineered capsid viral titers can be produced at titers comparable to the parental serotype, and can be measured, for example, by ddPCR.

It will be appreciated that while the different serotypes can provide some level of cell, tissue, and/or organ selectivity, each serotype still is multi-tropic and thus can result in tissue-toxicity for potential off target transduction. Furthermore, thermal stability among serotypes may vary significantly resulting in diminished vector efficacy. Thus, in addition to achieving some tissue targeting capacity and increased thermal stability via selecting an AAV of a particular serotype, it will be appreciated that thermal stability of the AAV serotype can be modified by an engineered AAV capsid described herein, and can be further modified with modifications, including peptide insertions, for example, to retarget tropism. As described elsewhere herein, variants of wild-type AAV of any serotype or other reference AAV strain can be generated via a method described herein and determined to have a particular cell-selective tropism, which can be the same or different as that of the reference wild-type AAV serotype or other reference AAV while having enhanced stability relative to the reference wild-type AAV serotype or other reference AAV strain.

The cell or tissue selectivity and/or thermal stability of the wild-type capsid or reference capsid can be enhanced (e.g., increased or diminished selectivity for a particular cell type that the parental wild-type serotype is biased towards and/or increased thermal stability of that serotype) in the engineered capsid. For example, wild-type AAV9 is capable of transducing muscle and cells in the human brain (see e.g., Srivastava. 2017. Curr. Opin. Virol. 21:75-80) and has a Tₘ of ˜77° C. (Bennett, A., et al. “Thermal Stability as a Determinant of AAV Serotype Identity.” Molecular Therapy: Methods & Clinical Development 2017, 6, 171-182). By including the engineered AAV capsid and/or capsid protein variant of wild-type AAV9 as described herein thermal stability of the viral capsid can be increased, thus enhancing the efficacy of the vector and/or the production yield or storage stability of the vector. Based on the improved thermal stability of the engineered capsid, further mutations and insertions that modify tropism or introduce resistance to neutralizing antibodies can be introduced that allow for improved stability relative to the same mutations introduced into a parental serotype capsid.

In one embodiment, the viral capsid protein may comprise one or more mutations relative to a wild type or reference capsid. In one embodiment, the one or more mutations are selected from the group consisting of N41D, G56F, V211M, Q233T, D349E, E361Q, D384N, E416T, N419D, K449R, Q456T, V465Q, Y478W, I479L, S483C, P504T, S507T, S508K, W509Y, A510H, E529D, G530D, H584L, Q597N, M640L, D665A, N668, and E712D in AAV9, or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid polypeptide.

In one embodiment, the mutations may comprise one or more mutations selected from the group consisting of N41D, G56F, V211M, Q233T, D384N, Y478W, I479L, S483C, P504T, S508K, W509Y, E529D, G530D, and M640L in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or mutations selected from the group consisting of N41D, G56F, V211M, Q233T, D384N, Y478W, I479L, S483C, P504T, S508K, W509Y, E529D, G530D, and M640L in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise N41D, G56F, V211M, Q233T, D384N, Y478W, I479L, S483C, P504T, S508K, W509Y, E529D, G530D, and M640L in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of D384N, S483C, P504T, S508K, W509Y, E529D, and G530D in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, P504T, S508K, W509Y, E529D, and G530D in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of D384N, S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations may comprise one or more mutations selected from the group consisting of D384N, S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of D384N, S483C, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of D384N, S483C, P504T, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, P504T, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations may comprise one or more mutations selected from the group consisting of D384N, S483C, P504T, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N, S483C, P504T, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise D384N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S483C in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise P504T in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S507T and/or S508K in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S507T, S508K, W509Y, A510H, or a combination thereof in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise S508K and/or W509Y in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the mutations comprise one or more mutations selected from the group consisting of E418D, R465Q, S467G, S507T, N510H, I517L, M541L, A568T, F577Y, F584L, H597N, M599Q, N642H, T665A, V699I, and P735N in AAV1 or in an analogous position in an AAV2, AAV3, AAV3B, AAV4, AAV5, AAV6, AAV7, AAV8, AAV9, AAV rh.74, AAV rh.8, AAV rh.10, or AAV-PHP.eB capsid polypeptide.

In one embodiment, the mutations comprise one or more mutations selected from the group consisting of S207G, M235L, V372I, N449Q, A467P, I470M, E499N, Y500F, S547A, A590P, V600A, S658P, and T713A in AAV2 or in an analogous position in an AAV1, AAV3, AAV3B, AAV4, AAV5, AAV6, AAV7, AAV8, AAV9, AAV rh.74, AAV rh.8, AAV rh.10, or AAV-PHP.eB capsid polypeptide.

In one embodiment, the mutations comprise one or more mutations selected from the group consisting of S205A, Q233T, K310R, V372I, L489V, A505G, S507T, H538S, N540V, E546Q, T548A, T549G, M648L, and T714A in AAV3B or in an analogous position in an AAV1, AAV2, AAV3, AAV3B, AAV4, AAV5, AAV6, AAV7, AAV8, AAV9, AAV rh.74, AAV rh.8, AAV rh.10, or AAV-PHP.eB capsid polypeptide.

In one embodiment, the mutations comprise one or more mutations selected from the group consisting of R465Q, S467G, S507T, N510H, I517L, K531E, M541L, A568T, F577Y, H597N, M599Q, T665A, V699I, and P735N in AAV6 or in an analogous position in an AAV1, AAV2, AAV3, AAV3B, AAV4, AAV5, AAV7, AAV8, AAV9, AAV rh.74, AAV rh.8, AAV rh.10, or AAV-PHP.eB capsid polypeptide.

In one embodiment, the mutations comprise one or more mutations selected from the group consisting of comprise V204M, T265S, S388A, S416T, Y466S, G468A, F486Y, K553N, L560M, P569T, F706Y, Q709S, and G711N in AAV7 or in an analogous position in an AAV1, AAV2, AAV3, AAV3B, AAV4, AAV5, AAV6, AAV8, AAV9, AAV rh.74, AAV rh.8, AAV rh.10, or AAV-PHP.eB capsid polypeptide.

In one embodiment, the mutations comprise one or more mutations selected from the group consisting of S224N, T415S, G468A, A507G, G508A, A520V, N540S, I542V, N549G, A551G, A555V, S667A, N670A, S712N in AAV8 or in an analogous position in an AAV1, AAV2, AAV3, AAV3B, AAV4, AAV5, AAV6, AAV7, AAV9, AAV rh.74, AAV rh.8, AAV rh.10, or AAV-PHP.eB capsid polypeptide.

In one embodiment, the mutations comprise one or more mutations selected from the group consisting of S224N, N263S, E330D, K333T, I343V, Q417T, T493K, S559N, A591T, V595T, D659N, L669F, and T722V in AAV rh.10 or in an analogous position in an AAV1, AAV2, AAV3, AAV3B, AAV4, AAV5, AAV6, AAV7, AAV8, AAV9, AAV rh.74, AAV rh.8, or AAV-PHP.eB capsid polypeptide.

In one embodiment, the mutations comprise one or more mutations selected from the group consisting of L389V, V479L, A507T, F509Y, K510H, D531E, G552N, L559M, A568T, N584L, H597N, I602L, and N668A in AAV rh.8 or in an analogous position in an AAV1, AAV2, AAV3, AAV3B, AAV4, AAV5, AAV6, AAV7, AAV8, AAV9, AAV rh.74, AAV rh.8, AAV rh.10, or AAV-PHP.eB capsid polypeptide.

In one embodiment, the viral capsid protein may consist of specific mutations relative to a wild type or reference capsid. In one embodiment, the specific mutations consist of N41D, G56F, V211M, Q233T, D384N, Y478W, I479L, S483C, P504T, S508K, W509Y, E529D, G530D, and M640L in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, P504T, S508K, W509Y, E529D, and G530D in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S483C, P504T, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S483C, P504T, S508K, W509Y, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, S508K, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, P504T, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N, S483C, P504T, W509Y, E529D, G530D, and Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of Q597N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of D384N in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S483C in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of P504T in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S507T and S508K in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S507T, S508K, W509Y, and A510H in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

In one embodiment, the specific mutations consist of S508K and W509Y in AAV9 or in an analogous position in an AAV1, AAV2, AAV3, AAV3B, AAV4, AAV5, AAV6, AAV7, AAV8, AAV rh.74, AAV rh.8, AAV rh.10, or AAV-PHP.eB capsid protein.

### Further Capsid Modifications

As described in more detail herein, two or more modifications, which individually may result in a minimal increase in efficiency or even a diminished efficiency, may result in a superior increase in efficiency when modified together. In one example embodiment, at least one modification to the AAV capsid selected from D384N, S483C, P504T, S508K, and W509Y with reference to AAV9 and at least one modification from E529D, G530D, Y478W, and I479L with reference to AAV9 result in increased efficiency. In one example embodiment, the modifications to the AAV capsid consisting of D384N, S483C, P504T, S508K, W509Y, E529D, and GG530D with reference to AAV9 result in increased efficiency. In one example embodiment, the modification to the AAV capsid protein is, at minimum, D384N, E529D and G530D with reference to AAV9 and results in increased efficiency. While the previously mentioned modifications are suggested for AAV9, analogous positions in AAV1, AAV2, AAV3, AAV3B, AAV4, AAV5, AAV6, AAV7, AAV8, AAV rh.74, AAV rh.8, AAV rh.10, and AAV-PHP.eB capsid polypeptide is also contemplated.

### Targeting Moieties

In example embodiments, the modified viral capsid protein comprises a targeting moiety with an enhanced tropism for one or more cell types. Advantageously, the engineered capsid proteins provide enhanced stability allowing for inclusion of targeting moieties without substantial loss to stability of the engineered AAV capsid, allowing an increase in tolerance to inclusion of the targeting moiety without compromising viral capsid protein folding. This targeting moiety may be coupled directly to a cargo to be delivered such as an oligonucleotide or polypeptide. Alternatively, the targeting molecule may be incorporated into a delivery particle to confer tropism for one or more cell type on the delivery particle. A non-limiting example of delivery particle is a viral capsid particle. In such embodiments, the targeting moiety may be incorporated into a viral capsid polypeptide such that the targeting moiety is incorporated into the assembled viral capsid. However, other particle delivery systems where the targeting moiety may be incorporated or attached, for example on exosomes or liposomes, are also envisioned and encompassed as alternative embodiments herein.

The targeting moiety can comprise a n-mer motif. As used herein, the term “n-mer motif” (used herein interchangeably with “n-mer peptide” and “n-mer”) refers to a peptide sequence consisting of a number of amino acid residues defined by the number “n.” For example, a 7-mer motif is a peptide sequence consisting of seven amino acid residues. In some embodiments, an n-mer motif may comprise 4, 5, 6, 7, 8, 9, 10, 11, 12, 13, 14, or 15 amino acid residues. In example embodiments, an n-mer motif consists of 7 amino acid residues. It should be understood that any reference to any amino acid is intended to encompass any natural amino acid as well as any amino acid mimetic having similar physical and chemical characteristics to naturally occurring amino acids.

In example embodiments, the targeting moiety can be used to increase transduction in target cells. The increase in transduction efficiency of the targeting moiety to a cell may be compared to a composition that does not contain the targeting moiety. For example, inclusion of one or more targeting moieties in a composition can result in an increase in transduction and or transduction efficiency by 10%, 20%, 30%, 40%, 50%, 60%, 70%, 80%, 90%, 100% or more. In an exemplary embodiment the increase in transduction and or transduction efficiency is one and a half fold, two-fold, three-fold, four-fold, five-fold, six-fold, seven-fold, eight-fold, nine-fold, ten-fold 20-fold 30-fold, 40-fold, 50-fold, 60-fold, 70-fold, 80-fold or more relative to a composition lacking the targeting moiety. In one embodiment the transduction and/or transduction efficiency is increased or enhanced in endothelial cells, in one embodiment increase in endothelial cells of the vasculature, for example, the central nervous system vasculature. In embodiments, the transduction and/or transduction efficiency is increased or enhanced in cells of the central nervous system. In embodiments, the transduction and/or transduction efficiency is increased or enhanced in hepatocytes or in endothelial cells of the kidney or of the muscle. In an embodiment, the composition comprising a targeting moiety is selective to a target cell as compared to other cell types and/or other virus particles. As used herein, ‘selective’ and ‘cell-selective’ refers to preferential targeting for cells as compared to other cell types. Preferably, the targeting moiety is selective for a desired target (e.g. cell, organ, system e.g. large diameter arteries and veins, brain, retina and spinal cord microvasculature, species) or set of targets by at least 2:1, 3:1, 4:1, 5:1, 6:1 7:1, 8:1, 9:1. 10:1 or more; or 10%, 20%, 30%, 40%, 50%, 60%, 70%, 75% 80%, 85%, 90% or more, relative to other targets or cells (e.g. microvasculature of peripheral organs such as the kidney). In an embodiment, the composition comprising a targeting moiety described herein can have an increased uptake, delivery rate, transduction rate, efficiency, amount, or a combination thereof in a target cell (e.g., endothelial cells across the arterio-venous axis in brain, retina, and spinal cord vasculature) as compared to other cells types (e.g., muscle cells) and/or other virus particles (e.g., AAVs not containing the targeting moiety) and other compositions that do not contain the cell-selective n-mer motif of the present invention.

In an example embodiment, targeting moieties disclosed herein can be inserted between two amino acids in the wild-type viral protein (VP) (or capsid protein). In some embodiments, the n-mer motif can be inserted between two amino acids in a variable amino acid region in a viral capsid protein.

The n-mer motif may be inserted between two amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parovirus capsids (see e.g., DiMattia et al. 2012. J. Virol. 86(12):6947-6958). Structural variable regions (VRs) occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In one example embodiment, one or more targeting moieties can be inserted between two amino acids in one or more of the 12 variable regions in the wild-type AVV capsid proteins. In one example embodiment, the one or more targeting motifs can be each be inserted between two amino acids in VR-I, VR-II, VR-III, VR-IV, VR-V, VR-VI, VR-VII, VR-III, VR-IX, VR-X, VR-XI, VR-XII, or a combination thereof. In one example embodiment, the engineered capsid is a modified AAV1 capsid and can have a n-mer motif inserted after or a neighbor of amino acid 590. In one example embodiment, the engineered capsid is a modified AAV3 capsid and can have a n-mer motif inserted after or a neighbor of amino acid 586. In one example embodiment, the engineered capsid is a modified AAV4 capsid and can have a n-mer motif inserted after or a neighbor of amino acid 586. In one example embodiment, the engineered capsid is a modified AAV5 capsid and can have a n-mer motif inserted after or a neighbor of amino acid 575. In one example embodiment, the engineered capsid is a modified AAV6 capsid and can have a n-mer motif inserted at or a neighbor of amino acid 585 and optionally Y705-731, T492V, K531E. In one example embodiment, the engineered capsid is a modified AAV8 capsid and can have a n-mer motif inserted after or a neighbor of amino acid 585 and 590. In one example embodiment, the engineered capsid is a modified AAV9 capsid and can have a n-mer motif inserted after or a neighbor of amino acid 588 and 589. (Büning, H.; Srivastava, A. Capsid Modifications for Targeting and Improving the Efficacy of AAV Vectors. Molecular Therapy—Methods & Clinical Development 2019, 12, 248-265). It will be appreciated that targeting moieties can be inserted in analogous positions in AAV viral proteins of other serotypes, such as but not limited to, AAV1, AAV2, AAV3, AAV4, AAV5, AAV6, AAV7, AAV8, AAV9, AAV rh.74, AAV rh.8, AAV rh.10, and AAV-PHP.eB capsid polypeptide. In some embodiments as previously discussed, the targeting moieties can be inserted between any two contiguous amino acids within the AAV viral protein and in some embodiments the insertion is made in a variable region.

In one example embodiment, the first 1, 2, 3, or 4 amino acids of an n-mer motif can replace 1, 2, 3, or 4 amino acids of a polypeptide into which it is inserted and preceding the insertion site. Using an AAV as another non-limiting example, one or more of the n-mer motifs can be inserted into e.g., an AAV9 capsid polypeptide between amino acids 588 and 589 and the insert can replace amino acids 586, 587, and 588 such that the amino acid immediately preceding the n-mer motif after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the targeting moiety is inserted are replaced by the targeting moiety.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

### Systems for Production of Modified Capsids

Also provided herein are expression systems that can contain one or more of the polynucleotides that can encode one or more of the engineered capsid proteins disclosed herein. As used in this context, engineered viral capsid polynucleotides refers to any one or more of the polynucleotides described herein capable of encoding an engineered viral capsid as described elsewhere herein and/or polynucleotide(s) capable of encoding one or more engineered viral capsid proteins described elsewhere herein. The engineered viral capsid polynucleotides may be comprised within one or more expression vectors. Further, where the expression vector includes an engineered viral capsid polynucleotide described herein, the vector can also be referred to and considered an engineered expression vector or system thereof although not specifically noted as such. In embodiments, the expression vector can contain one or more polynucleotides encoding one or more elements of an engineered viral capsid described herein. The expression vectors and systems thereof can be useful in producing bacterial, fungal, yeast, plant cells, animal cells, and transgenic animals that can express one or more components of the engineered viral capsid, particle, or other compositions described herein. Within the scope of this disclosure are expression vectors containing one or more of the polynucleotide sequences described herein. One or more of the polynucleotides that are part of the engineered viral capsid and system thereof described herein can be included in an expression vector or vector system.

The expression vector can include an engineered viral (e.g., AAV) capsid polynucleotide having a 3′ polyadenylation signal. In some embodiments, the 3′ polyadenylation is an SV40 polyadenylation signal. In some embodiments the expression vector does not have splice regulatory elements. In some embodiments, the expression vector includes one or more minimal splice regulatory elements. In some embodiments, the expression vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein. It some embodiments, the vector does not include one or more minimal splice regulatory elements, modified splice regulatory agent, splice acceptor, and/or splice donor.

The expression vectors and/or vector systems can be used, for example, to express one or more of the engineered viral (e.g., AAV) capsid and/or other polynucleotides in a cell, such as a producer cell, to produce engineered viral (e.g., AAV) particles and/or other compositions (e.g., polypeptides, particles, etc.) containing an engineered viral (e.g., AAV) capsid or other composition containing one or more modifications of the present invention described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. An expression vector can be a replicon, such as a plasmid, phage, or cosmid, into which another DNA segment may be inserted so as to bring about the replication of the inserted segment. Generally, an expression vector is capable of replication when associated with the proper control elements.

Expression vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of expression vector is a “plasmid,” which refers to a circular double stranded DNA loop into which additional DNA segments can be inserted, such as by standard molecular cloning techniques. Another type of an expression vector is a viral expression vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include adeno-associated viruses, and types of such vectors can also be selected for targeting particular types of cells, such as those engineered viral (e.g., AAV) vectors containing an engineered viral (e.g., AAV) capsid polynucleotide with a desired cell-selective tropism. These and other embodiments of the expression vectors and vector systems are described elsewhere herein.

In some embodiments, the expression vector can be a bicistronic vector. In some embodiments, a bicistronic expression vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter.

In addition to expression vectors that convey nucleic acid encoding the engineered capsid proteins and also rAAVs having an engineered capsid described herein and carry a payload transgene encoding, for example, a therapeutic polypeptide or nucleic acid or a detectable marker operably linked to a regularly sequence to promote expression in a target cell.

### Expression Vector Features

The expression vectors can include additional features that can confer one or more functionalities to the expression vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or expression vectors thereof described herein (including, but not limited to, the engineered AAV capsid polynucleotides of the present invention as well as transgenes encoding therapeutic polypeptides or polynucleotides or detectable markers) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, brain), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. For expression vectors encoding a transgene for packaging within the capsid, expression of the transgene may be under the control of the CAG promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the expression vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are discussed elsewhere herein and can be generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment).The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, the vector or system thereof can include one or more elements capable of translocating and/or expressing an engineered polynucleotide of the present invention (e.g., an engineered viral (e.g.,AAV) capsid polynucleotide or a transgene encoding a therapeutic polypeptide or polynucleotide or detectable marker) to/in a specific cell component or organelle. Such organelles can include, but are not limited to, nucleus, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

The expression systems for producing modified capsid proteins and capsid (e.g., an engineered viral (e.g., AAV) capsid polynucleotide) can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide of the present invention (e.g., an engineered viral (e.g., AAV) capsid polynucleotide) such that the selectable marker polypeptide, when translated, is inserted between two amino acids between the N- and C-terminus of an engineered polypeptide (e.g., the engineered AAV capsid polypeptide) or at the N- and/or C-terminus of the engineered polypeptide (e.g., an engineered AAV capsid polypeptide). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the expression system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system or other compositions and/or systems described herein via suitable linker, such as a glycine or glycine serine linkers as short as GS or GG up to (GGGGG)₃ (SEQ ID NO: 25) or (GGGGS)₃ (SEQ ID NO: 26). Other suitable linkers are described elsewhere herein.

The expression vector or expression vector system can include one or more polynucleotides encoding one or more targeting moieties. In some embodiments, the targeting moiety encoding polynucleotides can be included in the expression vector or expression vector system, such as a viral vector system, such that they are expressed within and/or on the virus particle(s) produced such that the virus particles can be targeted to selective cells, tissues, organs, etc. In some embodiments, the targeting moiety encoding polynucleotides can be included in the expression vector or expression vector system such that the engineered polynucleotide(s) of the present invention (e.g., an engineered viral (e.g., AAV) capsid polynucleotide(s)) and/or products expressed therefrom include the targeting moiety and can be targeted to selective cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered polynucleotide(s) of the present invention, the engineered polypeptides, or other compositions of the present invention described herein, to select cells, tissues, organs, etc. In some embodiments, the select cells are muscle cells.

### Cell-Free Vector and Polynucleotide Expression

In some embodiments, the polynucleotide encoding one or more features of the engineered AAV capsid system can be expressed from an expression vector or suitable polynucleotide in a cell-free in vitro system. In other words, the polynucleotide can be transcribed and optionally translated in vitro. In vitro transcription/translation systems and appropriate expression vectors are generally known in the art and commercially available. Generally, in vitro transcription and in vitro translation systems replicate the processes of RNA and protein synthesis, respectively, outside of the cellular environment. Expression vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenolpyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Polynucleotides Encoding the Capsid Proteins or Cargo Molecules

As described elsewhere herein, the polynucleotide encoding an engineered capsid of the present invention and/or other polynucleotides described herein can be codon optimized. In some embodiments, polynucleotides of the engineered AAV capsid system described herein can be codon optimized. In some embodiments, one or more polynucleotides contained in a vector (“vector polynucleotides”) described herein that are in addition to an optionally codon optimized polynucleotide encoding the engineered capsid proteins, including but not limited to, embodiments of the engineered AAV capsid system described herein, can be codon optimized. In addition, in embodiments, the transgene can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, and Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; and Selection on the codon bias of chloroplast and cyanelle genes in different plant and algal lineages, Morton B R, J Mol Evol. 1998 April; 46(4):449-59.

The expression vector polynucleotide can be codon optimized for expression in a select cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. In some embodiments, the polynucleotide is codon optimized for a specific cell type or types. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, an expression vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Modified Viral Delivery Capsid

As used herein a “delivery vector” or “viral vector” is in reference to the rAAV capsids comprising the engineered capsid proteins described herein and used to deliver a cargo such a transgene encoding a therapeutic polypeptide, nucleic acid, or detectable marker. The viral delivery vector can be part of a viral vector system involving multiple delivery vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral or adeno-associated viral (AAV) vector. In some embodiments, the adenoviral vector can include elements such that the virus particle produced using the vector or system thereof is an AAV and can be serotype AAV1, AAV2, AAV3, AAV3B, AAV4, AAV5, AAV6, AAV7, AAV8, AAV rh.74, AAV rh.8, AAV rh.10 capsid. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261. The engineered AAV capsids can be included in an adenoviral vector to produce adenoviral particles containing said engineered AAV capsids.

In one embodiment the vector can be a helper-dependent adenoviral vector or system thereof. These are also referred to in the field as “gutless” or “gutted” vectors and are a modified generation of adenoviral vectors (see e.g., Thrasher et al. 2006. Nature. 443:E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In one embodiment, the polynucleotide to be delivered via the viral particle produced from a helper-dependent adenoviral vector or system thereof can be up to about 38 kb. Thus, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In one embodiment, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered AAV capsid system of the present invention.

### Adeno Associated Viral Vectors

In an embodiment, the engineered vector or system thereof can be an adeno-associated viral vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific or preferred site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof. In some embodiments, the promoter can be a tissue specific promoter as previously discussed. In some embodiments, the tissue specific promoter can drive expression of a transgene or cargo polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-selective or non-selective tropism and have increased thermal stability relative to a reference capsid as described further herein.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce engineered AAV particles having a specific serotype. In some embodiments, the serotype can be AAV1, AAV2, AAV3, AAV3B, AAV4, AAV5, AAV6, AAV8, AAV9 or any combinations thereof. In some embodiments, the AAV can be AAV1, AAV2, AAV5, AAV9 or any combination thereof. One can select the AAV with regard to the cells to be targeted; e.g., one can select AAV serotypes 1, 2, 5, 9 or a hybrid capsid AAV1, AAV2, AAV5, AAV9 or any combination thereof for targeting brain and/or neuronal cells; and one can select AAV4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV1, AAV2, AAV5 or any combination thereof. In some embodiments, an AAV vector or system thereof capable of producing AAV particles capable of targeting cardiac tissue can be configured to generate an AAV particle having an AAV4 serotype. In some embodiments, an AAV vector or system thereof capable of producing AAV particles capable of targeting the liver can be configured to generate an AAV having an AAV8 serotype. See also Srivastava. 2017. Curr. Opin. Virol. 21:75-80. The AAV systems as described herein have one or more amino acid substitutions that increase the thermal stability relative to the wild type or reference capsid.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same selectivity issues as with the non-hybrid wild-type serotypes previously discussed (Castle, M. J., et al. Controlling AAV Tropism in the Nervous System with Natural and Engineered Capsids. In Gene Therapy for Neurological Disorders; Springer New York, 2016; pp 133-149), and can use engineered capsid proteins as detailed herein to improve stability.

Advantages achieved by the wild-type based hybrid AAV systems can be combined with the engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV9 serotype that is used to package a genome that contains components from an AAV2 serotype. Such components may include AAV2 inverted terminal repeats (ITRs) flanking the transgene and regulatory elements of the packaged genome. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82: 5887-5911 (2008) reproduced below as Table 2. Further tropism details can be found in Srivastava. 2017. Curr. Opin. Virol. 21:75-80 as previously discussed.

TABLE 2

| Cell Line | AAV1 | AAV2 | AAV3 | AAV4 | AAV5 | AAV6 | AAV8 | AAV9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In one example embodiment, the AAV vector or system thereof is AAV1, AAV2, AAV3, AAV3B, AAV4, AAV5, AAV6, AAV7, AAV8, AAV9, AAV rh.74, AAV rh.8, or AAV rh.10, AAV-PHP.eB and comprises the one or more amino acid substitutions which increase the thermal stability of the capsid relative to the wild type or reference capsid.

In another example embodiment, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., a transgene encoding a therapeutic polypeptide or polynucleotide or a detectable marker).

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of a engineered AAV capsid system described herein are as used in the foregoing documents, such as WO 2014/093622 (PCT/US2013/074667) and are discussed in greater detail herein

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., a transgene encoding a polypeptide or polynucleotide of interest, including for therapeutic use, or a detectable marker operably linked to a regulatory element that promotes transgene expression in a target cell) between 2 ITRs; (2) a vector that carries the AAV Rep-Cap encoding polynucleotides; including the polynucleotide encoding the engineered capsid proteins described herein; and (3) helper polynucleotides encoding the adenoviral factors necessary for AAV production. One of skill in the art will appreciate various methods and variations thereof that are both helper and -helper free and as well as the different advantages of each system.

The engineered AAV expression vectors and systems thereof described herein can be produced by any of these methods.

A delivery vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

The engineered AAV delivery vectors and systems can be used to deliver a polynucleotide comprising a transgene encoding a therapeutic polypeptide or polynucleotide or a detectable marker in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

### Engineered Virus Particles Including an Engineered Viral (e.g., AAV) Capsid

Also described herein are engineered virus particles (also referred to here and elsewhere herein as “engineered viral particles” that can contain an engineered viral capsid (e.g., AAV capsid, referred to as “engineered AAV” or “rAAV”) as described in detail elsewhere herein. It will be appreciated that the engineered AAV particles can be adenovirus-based particles, helper adenovirus-based particles, AAV-based particles, or hybrid adenovirus-based particles that contain at least one engineered AAV capsid proteins as previously described. An engineered AAV capsid is one that that contains one or more engineered AAV capsid proteins as are described elsewhere herein. The engineered AAV particles can thus include one or more targeting moieties previously described.

The engineered AAV particle can include one or more cargo molecules. Engineered AAV particles can be provided in formulations, detailed elsewhere herein. Methods of making the engineered AAV particles from vectors are also described.

### Cargo

Cargo that can be associated with or packaged within the engineered AAV particles can comprise a recombinant AAV genome which comprises a transgene encoding one or more polypeptides, polynucleotides, or ribonucleoprotein complex. In an embodiment, the polynucleotide encodes one or more polypeptides and/or a short or small hairpin RNA (shRNA) or a microRNA (miRNA). In an embodiment, the polynucleotide encodes one or more polypeptides. In an embodiment, the one or more polypeptides comprise enzymes, transport proteins or antibodies. In an embodiment, the polynucleotide encodes a CRISPR-Cas.

The one or more cargo polynucleotides are packaged into an engineered viral (e.g., AAV) particle, which can be delivered to, e.g., a cell.

In some embodiments, the cargo polynucleotide encodes a product that is capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

In one example embodiment, the cargo molecule is a polynucleotide that is or can encode a vaccine. In another example embodiment, the cargo molecule is a polynucleotide encoding an antibody.

In some embodiments, the cargo is a cargo polynucleotide that can be packaged into an engineered viral particle and subsequently delivered to a cell. In some embodiments, delivery is cell selective, e.g., endothelial cell of the central nervous system vasculature. In example embodiments, delivery is not cell selective, e.g., selectivity is expanded beyond the typical tropism for a serotype. The engineered viral (e.g., AAV) capsid polynucleotides, other viral (e.g., AAV) polynucleotide(s), and/or vector polynucleotides can contain one or more cargo polynucleotides. In some embodiments, the cargo polynucleotide encodes a polynucleotide that is capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

In some embodiments, the cargo molecule encodes a polynucleotide or polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc., are other non-limiting examples of gene modification systems whose one or more components can be delivered by the engineered viral (e.g., AAV) particles described herein.

In some embodiments, the cargo polynucleotide encodes a molecule which is a gene editing system or component thereof. In some embodiments, the cargo molecule is a CRISPR-Cas system molecule or a component thereof. In some embodiments, the cargo molecule is a polynucleotide that encodes one or more components of a gene modification system (such as a CRISPR-Cas system). In some embodiments the cargo molecule is a gRNA. CRISPR-Cas system as used herein is intended to encompass by Class 1 and Class 2 CRISPR-Cas systems and derivatives of CRISPR-Cas systems such as base editors, prime editors, and CRISPR-associated transposases (CAST) systems.

An embodiment of the invention encompasses methods of modifying a genomic locus of interest to change gene expression in a cell by introducing into the cell any of the compositions described herein.

An embodiment of the invention is that the above elements are comprised in a single composition or comprised in individual compositions. These compositions may advantageously be applied to a host to elicit a functional effect on the genomic level.

### Gene Modification by Cargo Polynucleotides and Products

In some embodiments, the cargo molecule can encode a polynucleotide or polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc. are other non-limiting examples of gene modification systems whose one or more components can be delivered by the engineered viral (e.g., AAV) particles described herein.

In some embodiments, the cargo molecule encodes a gene editing system or component thereof. In some embodiments, the cargo molecule encodes a CRISPR-Cas system molecule or a component thereof. In some embodiments, the cargo molecule encodes a polynucleotide that encodes one or more components of a gene modification system (such as a CRISPR-Cas system). In some embodiments the cargo molecule is a gRNA. CRISPR-Cas system as used herein is intended to encompass by Class 1 and Class 2 CRISPR-Cas systems and derivatives of CRISPR-Cas systems such as base editors, prime editors, and CRISPR-associated transposases (CAST) systems.

An embodiment of the invention encompasses methods of modifying a genomic locus of interest to change gene expression in a cell by introducing into the cell any of the compositions described herein.

An embodiment of the invention is that the above elements are comprised in a single composition or comprised in individual compositions. These compositions may advantageously be applied to a host to elicit a functional effect on the genomic level.

Engineered Cells and Organisms Expressing said Engineered AAV Capsids

Described herein are engineered cells that can include one or more of the engineered AAV capsid polynucleotides, polypeptides, vectors, and/or vector systems. In some embodiments, one or more of the engineered AAV capsid polynucleotides can be expressed in the engineered cells. In some embodiments, the engineered cells can be capable of producing engineered AAV capsid proteins and/or engineered AAV capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered AAV capsid polynucleotide as described elsewhere herein.

A wide variety of animals, plants, algae, fungi, yeast, etc. and animal, plant, algae, fungus, yeast cell or tissue systems may be engineered to express one or more nucleic acid constructs of the engineered AAV capsid system described herein using various transformation methods mentioned elsewhere herein. This can produce organisms that can produce engineered AAV capsid particles, such as for production purposes, engineered AAV capsid design and/or generation, and/or model organisms. In some embodiments, the polynucleotide(s) encoding one or more components of the engineered AAV capsid system described herein can be stably or transiently incorporated into one or more cells of a plant, animal, algae, fungus, and/or yeast or tissue system. In some embodiments, one or more of engineered AAV capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered AAV capsid system described herein are expressed in one or more cells of the plant, animal, algae, fungus, yeast, or tissue systems.

### Engineered Cells

Described herein are various embodiments of engineered cells that can include one or more of the engineered AAV capsid system polynucleotides, polypeptides, vectors, and/or vector systems described elsewhere herein. In some embodiments, the cells can express one or more of the engineered AAV capsid polynucleotides and can produce one or more engineered AAV capsid particles, which are described in greater detail herein. Such cells are also referred to herein as “producer cells”. It will be appreciated that these engineered cells are different from “modified cells” described elsewhere herein in that the modified cells are not necessarily producer cells (i.e., they do not make engineered GTA delivery particles) unless they include one or more of the engineered AAV capsid polynucleotides, engineered AAV capsid vectors or other vectors described herein that render the cells capable of producing an engineered AAV capsid particle. Modified cells can be recipient cells of an engineered AAV capsid particles and can, in some embodiments, be modified by the engineered AAV capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered AAV capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of AAV.

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue(DE3), BLR, C41(DE3), C43(DE3), Lemo21(DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered cell can be a fungal cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerervisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella 62sabelline).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific or selective regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when a engineered AAV capsid particle is produced it can package one or more cargo polynucleotides that can be related to the desired physiological and/or biological characteristic and/or capable of modifying the desired physiological and/or biological characteristic. Thus, the cargo polynucleotides of the produced engineered AAV capsid particle can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered viral (e.g., AAV) capsid polynucleotides, vectors, and/or particles. In some embodiments, the engineered viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more selective targeting moieties described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 pg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 pg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, L, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered AAV capsid particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-a, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatories (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for oral administration can be discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as foam, spray, or liquid solution. In some embodiments, the oral dosage form can contain about 1 ng to 1000 g of a pharmaceutical formulation containing a therapeutically effective amount or an appropriate fraction thereof of the targeted effector fusion protein and/or complex thereof or composition containing the one or more of the polypeptides, polynucleotides, vectors, cells, and combinations thereof described herein. The oral dosage form can be administered to a subject in need thereof.

Where appropriate, the dosage forms described herein can be microencapsulated.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for parenteral administration and/or adapted for any type of injection (e.g., intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### Kits

Also described herein are kits that contain one or more of the one or more of the compositions, polypeptides, polynucleotides, vectors, cells, or other components described herein and combinations thereof and pharmaceutical formulations described herein. In embodiments, the kits comprise the engineered viral particles as detailed herein, and can comprise additional instructions for use of the stabilized particles for further manipulations, including additional mutations. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered polynucleotides, such as those containing a selective targeting moiety, as described elsewhere herein and, optionally, a cargo molecule, which can optionally be operably linked to a regulatory element. The one or more engineered polynucleotides such as those containing a selective targeting moiety, as described elsewhere herein and, can be included on the same or different vectors as the cargo molecule in embodiments containing a cargo molecule within the kit.

### Methods of Use

The engineered AAV particles detailed herein can be utilized in methods of delivering cargos to cells. Methods can comprise administering an engineered AAV particle as detailed herein to a population of cells. The engineered particle is loaded with a cargo and the capsid comprises one or more n-mer peptides that alter or further refine a tropism of the engineered AAV particle. The compositions including engineered AAV capsid particles, which optionally comprise one or more of cell targeting moieties can be used generally to package and/or deliver one or more cargos to one or more cell types. In some embodiments, delivery is done in cell-indiscriminate manner based upon the promiscuity of the targeting moiety. In some embodiments this is conferred by the tropism of the engineered AAV capsid, which can be influenced at least in part by the inclusion of one or n-mer motifs described elsewhere herein. In some embodiments, compositions including engineered AAV capsid particles, optionally one or more cell-selective moieties, can be administered to a subject or a cell, tissue, and/or organ and facilitate the transfer of the cargo polypeptide to the recipient cell. In other embodiments, engineered cells capable of producing compositions, such as polypeptides and other particles (e.g., engineered AAV capsids and viral particles), containing one or more of the targeting moieties can be generated from the polynucleotides, vectors, and vector systems etc., described herein. This includes without limitation, the engineered AAV capsid system molecules (e.g., polynucleotides, vectors, and vector systems, etc.). In some embodiments, the polynucleotides, vectors, and vector systems etc., described herein capable of generating the compositions, such as particles (e.g., engineered AAV capsids and viral particles), optionally containing one or more of the targeting moieties can be delivered to a cell or tissue, in vivo, ex vivo, or in vitro. In some embodiments, when delivered to a subject, the composition can transform a subject's cell in vivo or ex vivo to produce an engineered cell that can be capable of making a composition described herein that contains one or more of the cell-selective targeting moieties described herein, including but not limited to the engineered AAV capsid particles, which can be released from the engineered cell and deliver cargo molecule(s) to a recipient cell in vivo or produce personalized engineered compositions (e.g., AAV capsid particles) for reintroduction into the subject from which the recipient cell was obtained.

In some embodiments, the compositions, such as polypeptides and other particles (e.g., engineered AAV capsids and viral particles), optionally containing one or more of the targeting moieties, can be delivered to cells or tissues.

The engineered AAV capsids may be used to deliver a variety of therapeutic payloads (cargos) in a variety disease contexts. To date, AAV vectors have been evaluated in delivering therapeutic payloads in 136 clinical trials. Au et al. Gene Therapy Advances: A Meta-Analysis of AAV Usage in Clinical Settings Front Med (Lausanne), 2022 Feb. 9 doi: 10.3389/fmed.2021.809118. The improved product, stability, and transfection efficiency of the engineered AAV capsids disclosed herein may be used in methods to deliver similar therapeutic cargos for similar therapeutic purposes. The engineered AAV capsid disclosed herein may be used in methods for gene replacement, gene addition, gene silencing, and gene editing. The improved AAV capsids disclosed herein may be used in methods of delivering therapeutic cargos for treatment of blood disorders, central nervous system disorders, eye disorder, lysosomal storage disorder, and neuromuscular disorders. The engineered AAV capsids may also be used in delivering therapeutic cargos for treatment of various forms of cancer. Therapeutic cargos may encompass transgenes that activate tumor suppressors (e.g., PTEN, TP53), silence oncogenes (e.g., MYCN, MYC, WNTs), induce cell death (e.g., TRAIL, FASL, miR26a, HSV1-TK), induce cell cycle arrest (e.g., CDKs, cyclins, miR-122, MIS), prevent angiogenesis (e.g., bevacizumab), or mount a target immune response against tumors. The engineered AAV capsids may also be used for development of adoptive cell therapies such as CAR T, CAR NK, and tumor infiltrating lymphocytes (TILs).

In some embodiments, the engineered AAV capsid polynucleotides, vectors, and systems thereof can be used to generate engineered AAV capsid variant libraries that can be mined for variants with a desired cell-selectivity. The description provided herein as supported by the various Examples can demonstrate that one having a desired cell-selectivity in mind could utilize the present invention as described herein to obtain a capsid with the desired cell-selectivity while retaining transduction efficiency, viral titers, increased stability, and other advantages detailed herein.

Further embodiments are illustrated in the following Examples which are given for illustrative purposes only and are not intended to limit the scope of the invention.

### EXAMPLES

### Example 1

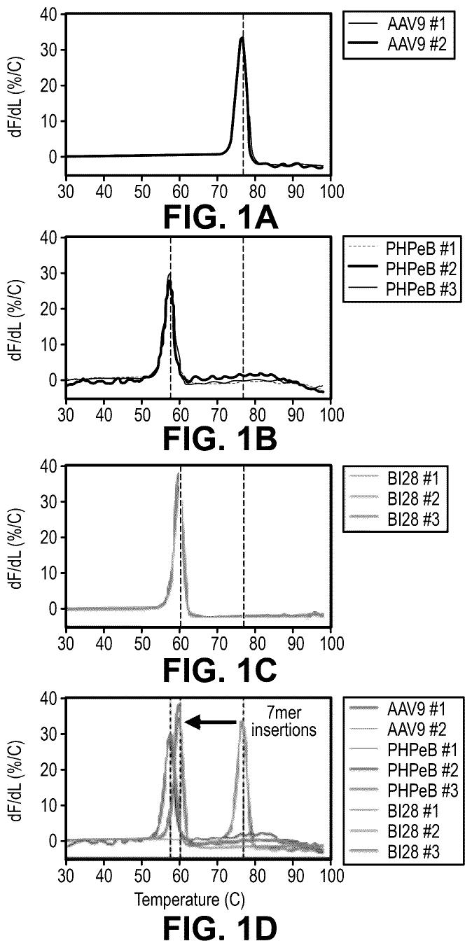

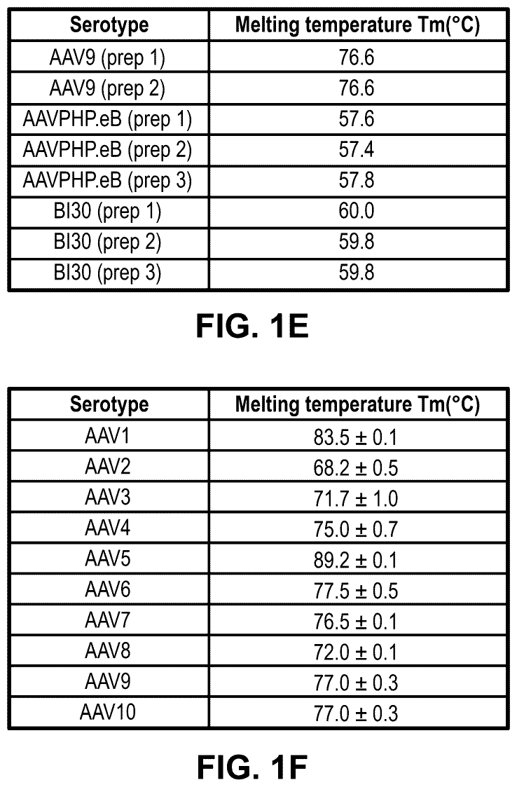

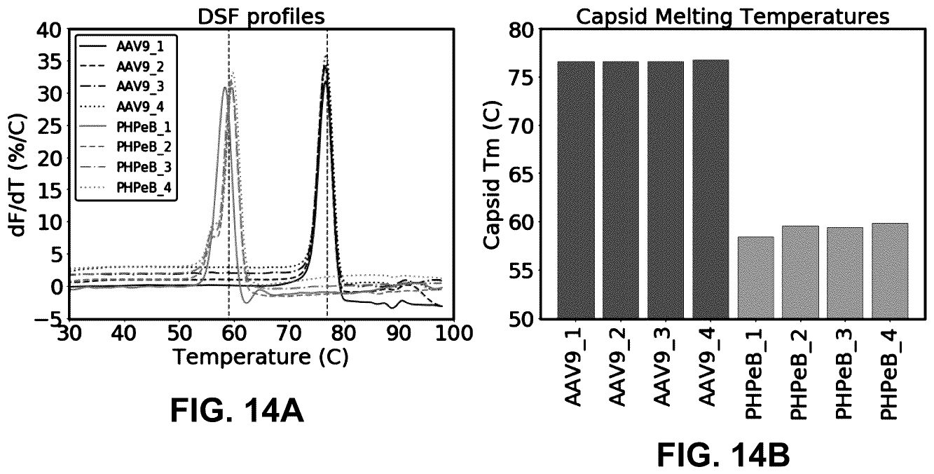

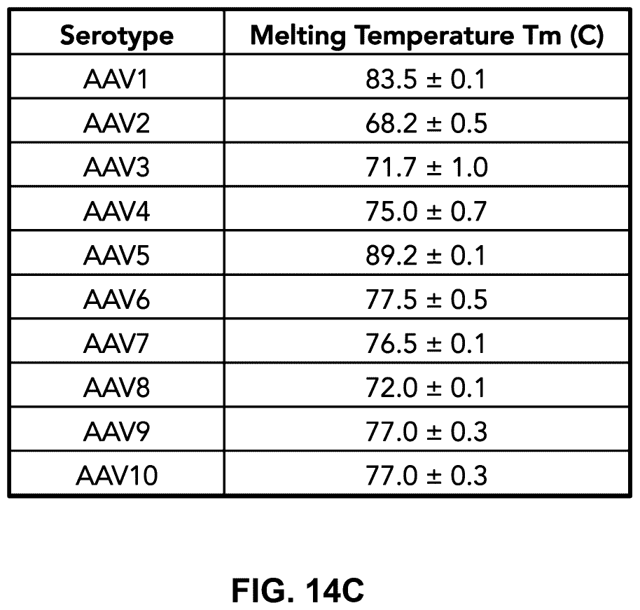

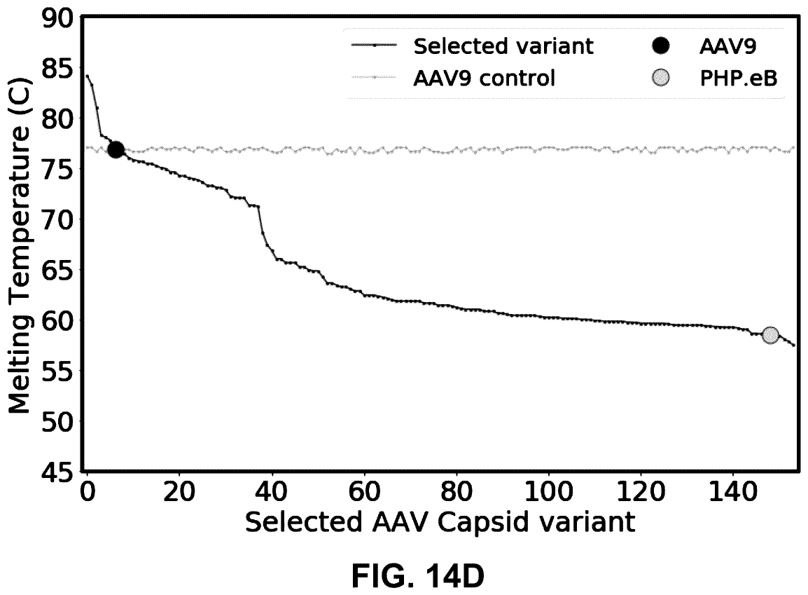

Modifying AAV capsids through targeted 7-mer peptide insertions is a proven strategy for engineering AAVs with unique properties (Dalkara et al. In vivo-directed evolution of a new adeno-associated viruses for therapeutic outer retinal gene delivery from the vitreous Sci Transl Med. 2013 12:5(189); Deverman et al. CRE-dependent selection yields AAV variants for widespread gene transfer to the adult brain Nat Biotechnol. 2016 34(2):204-9; Chan et al. Engineered AAVs for efficient noninvasive gene delivery to the central and peripheral nervous systems Nat Neurosci. 2017 20(8):1172-1179; Tervo et al. A designer AAV variant permits efficient retrograde access to projection neurons Neuron 2016, 19:92(2):372-382; Hanlon et al. Selection of an efficient AAV vector for robust CNS transgene expression. Mol Ther Methods Clin Dev. 2019 15:320-332; Nonnenmacher et al. Rapid evolution of blood-brain-barrier-penetrating AAV capsids by RNA-driven biopanning Mol Ther Methods Clin Dev. 2020 20:366-378). However, Applicants found that 7-mer insertions can dramatically reduce the thermal stability of the AAV9 capsid. For example, AAV-PHP.eB and BI28, two synthetic variants generated through insertion of a 7-mer peptide in the loop VIII of AAV9 VP3 protein, were subjected to differential scanning fluorimetry (DSF) (FIGS. 1A-1E and 14A). AAV-PHP.eB denatured at a temperature of 59.3° C., 17.35° C. lower than its parental capsid AAV9 (FIGS. 1A-1E and 14A-14B), and below the denaturation temperature of any naturally occurring serotype subjected to DSF to date (Rayaprolu et al 2013, Pacouret et al 2016, Bennett et al 2017) (FIGS. 1F and 14C). In addition, the Applicants subjected a total of 153 AAV9-derived variants, engineered via substitutions and/or 7-mer peptide insertion, to DSF, and found that 147 of them had a lower capsid Tm than AAV9 (FIG. 14D). This indicated that AAV engineering via this proven approach can destabilize the AAV capsid. Modifications strongly destabilizing to the AAV capsid, such as 7-mer peptide insertion in loop VIII, could be too destabilizing and poorly tolerated. Furthermore, the destabilization caused by 7-mer insertions may limit the introduction of additional beneficial substitutions, insertions, or deletions at other sites within the capsid. Therefore, the Applicants reasoned that stabilizing the AAV9 capsid could increase the tolerance to insertions, deletions, and substitutions, without compromising VP protein folding, capsid assembly, and DNA packaging.

Consensus mutagenesis is a sequence-based approach that has been used by several groups to improve the thermodynamic stability of proteins (Godoy-Ruiz et al 2006, Bershtein et al 2008). Singleton mutations tend to be destabilizing. One consequence of this observation is that, mutating the singleton residues of a protein to the consensus amino acids, determined through a multiple sequence alignment of homologous proteins, often lead to an increase in thermal stability. This phenomenon was actually postulated to be responsible for the increased thermal stability observed for putative ancestral proteins predicted using ASR, as maximum likelihood methods tend to eliminate rare mutations, such as destabilizing ones (Williams, Pollock et al, 2006).

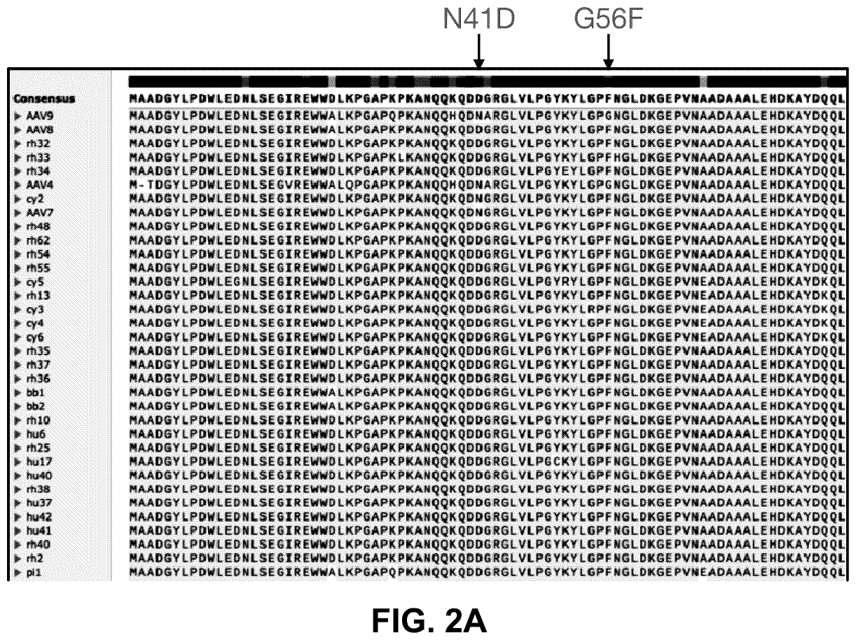

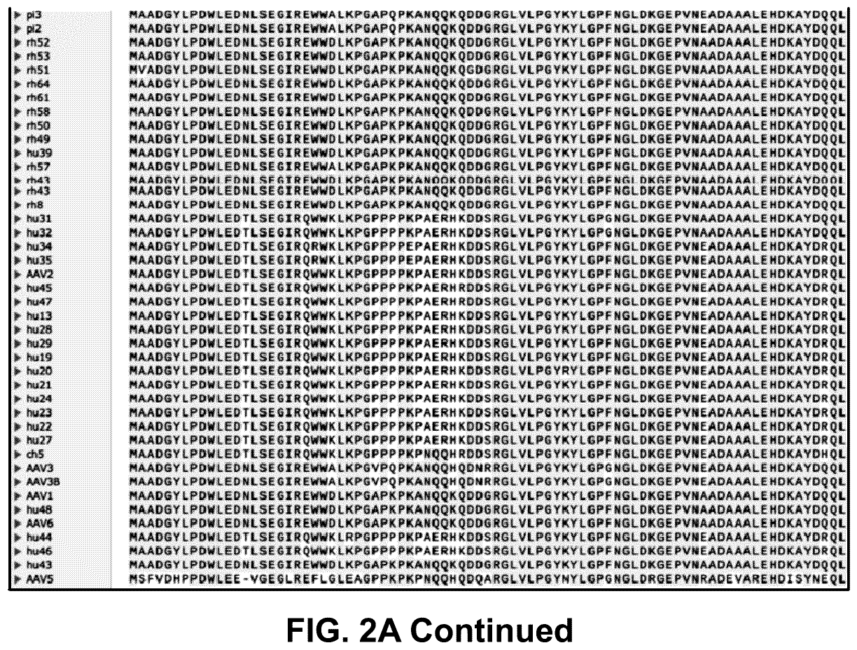

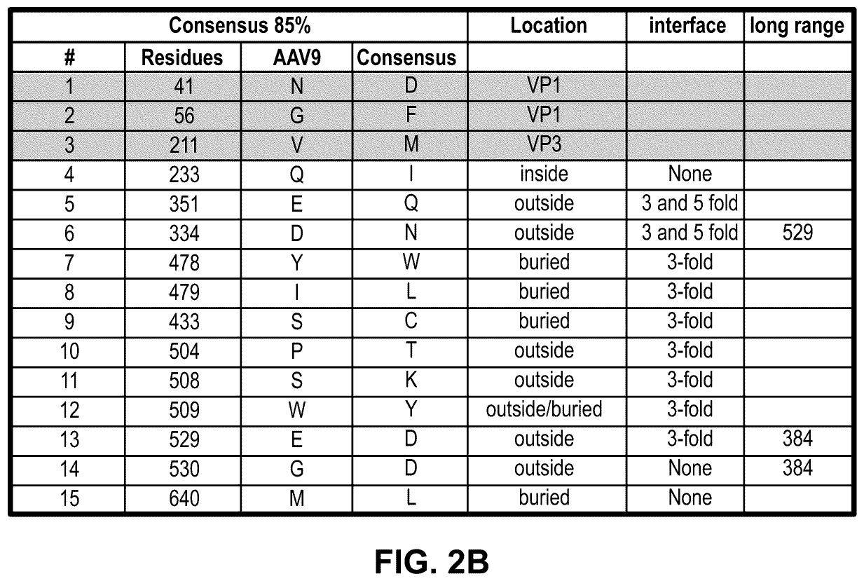

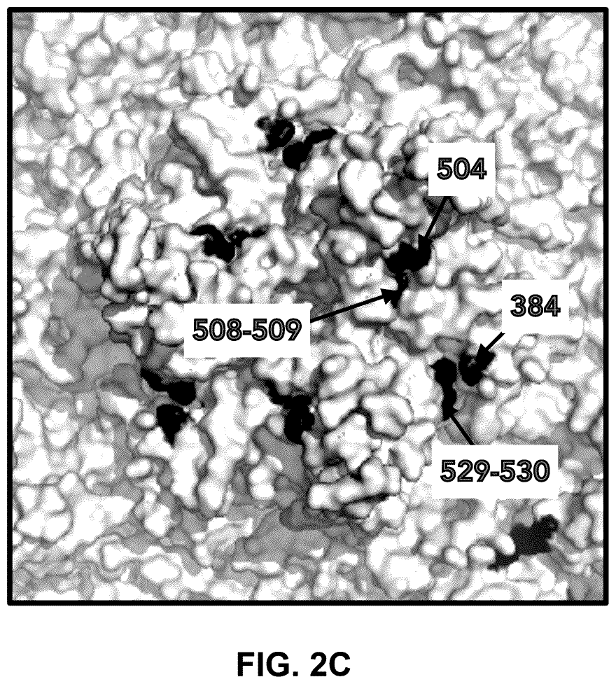

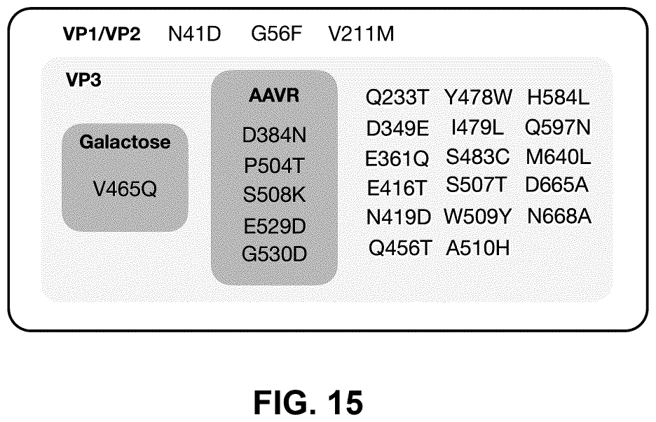

Consensus mutagenesis was applied to AAV9. A consensus sequence was generated from the multiple sequence alignment of the VP1 capsid proteins of 75 naturally occurring serotypes (Zinn et al 2015), using a conservation threshold of 75% (FIG. 2A). 26 divergent residues were further identified between AAV9 and the consensus sequence (FIGS. 2B and 15). Three residues appeared to be within VP1 unique N-terminal domain, whereas other residues were scattered across the VP3 protein chain. In addition, residue 465 was found to be part of the galactose binding footprint, whereas residues 384, 504, 508, 529, and 530 were part of the AAVR binding footprint of the AAV9 capsid (FIGS. 2B and 15). Interestingly, residues 529 and 384 were shown to be in close proximity in VP3 folded structure (FIG. 2C).

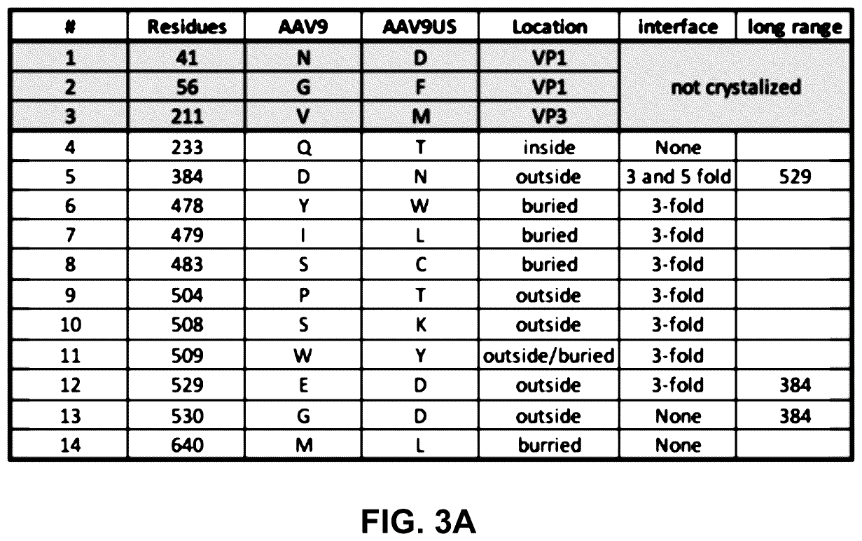

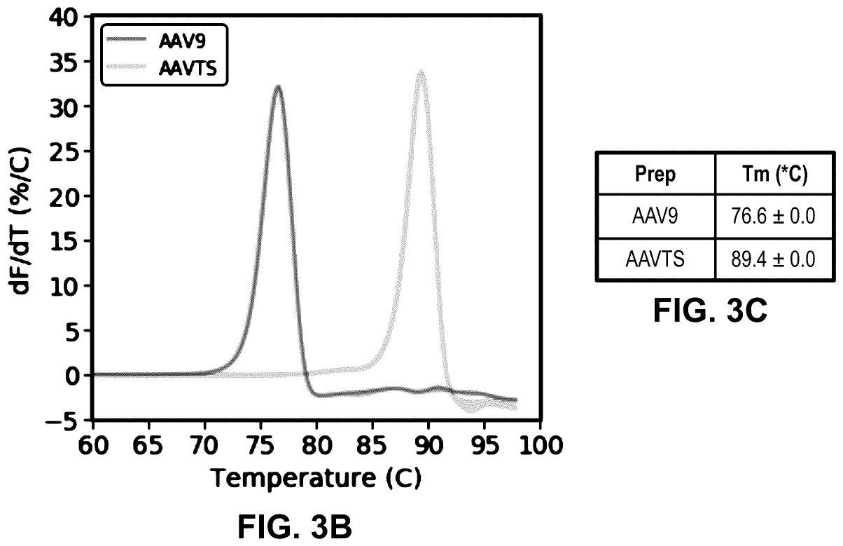

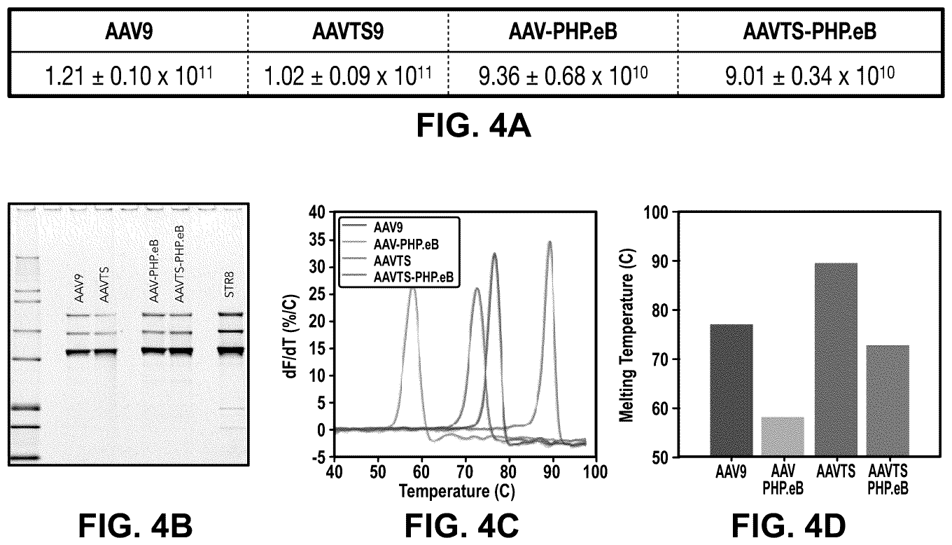

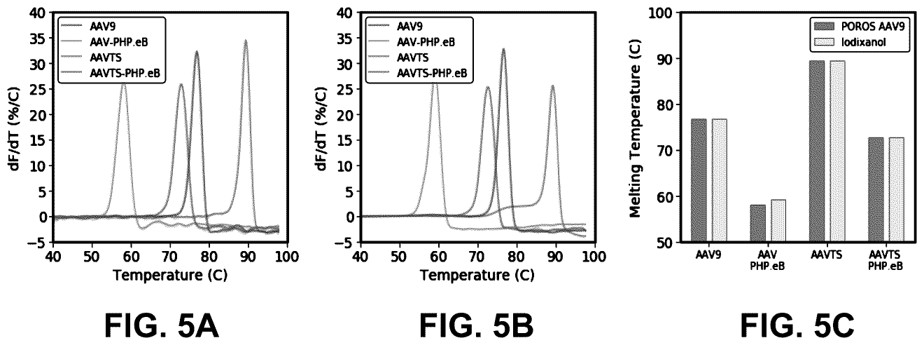

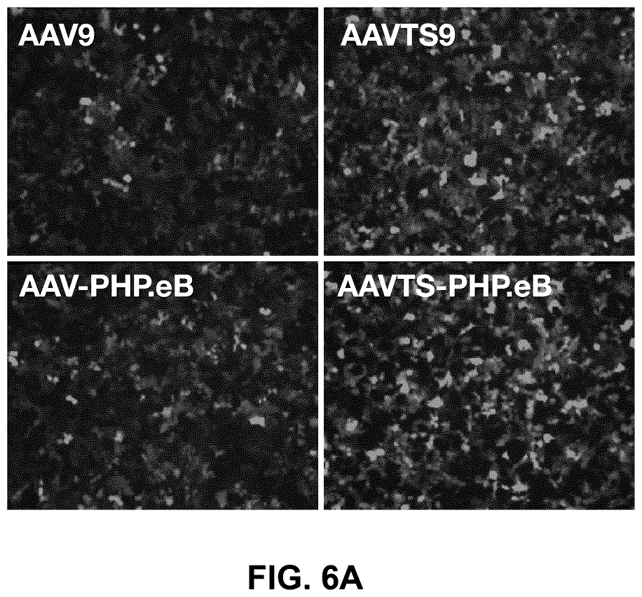

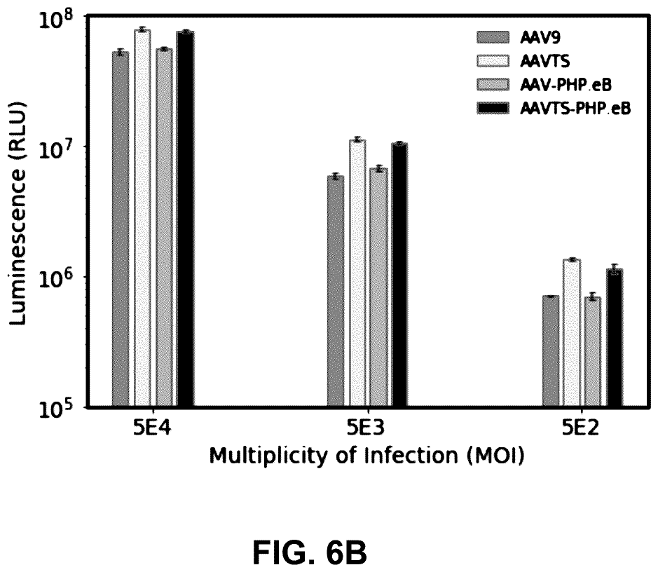

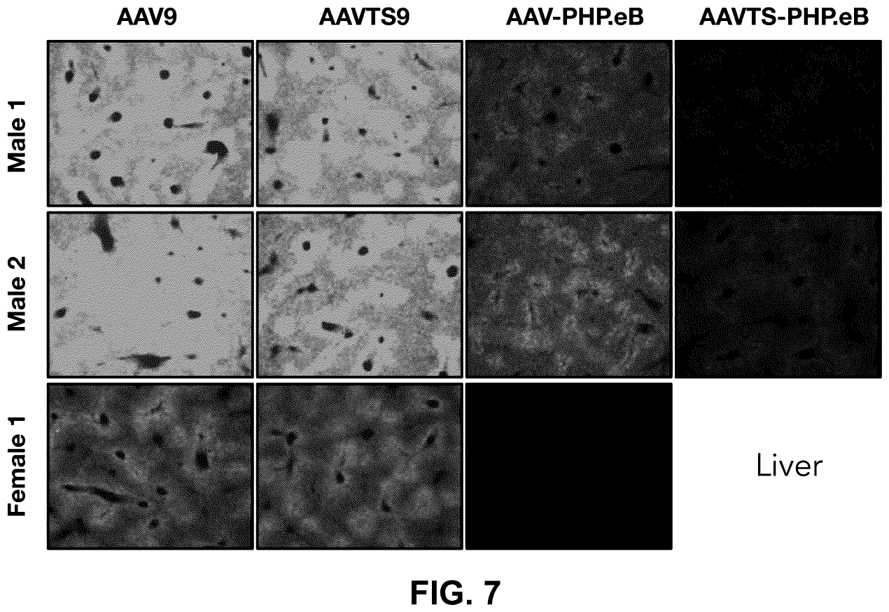

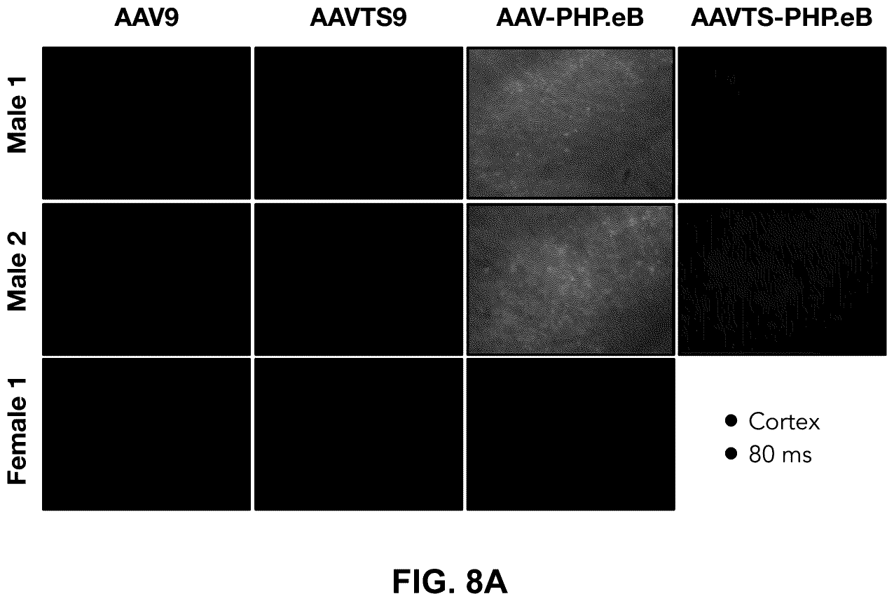

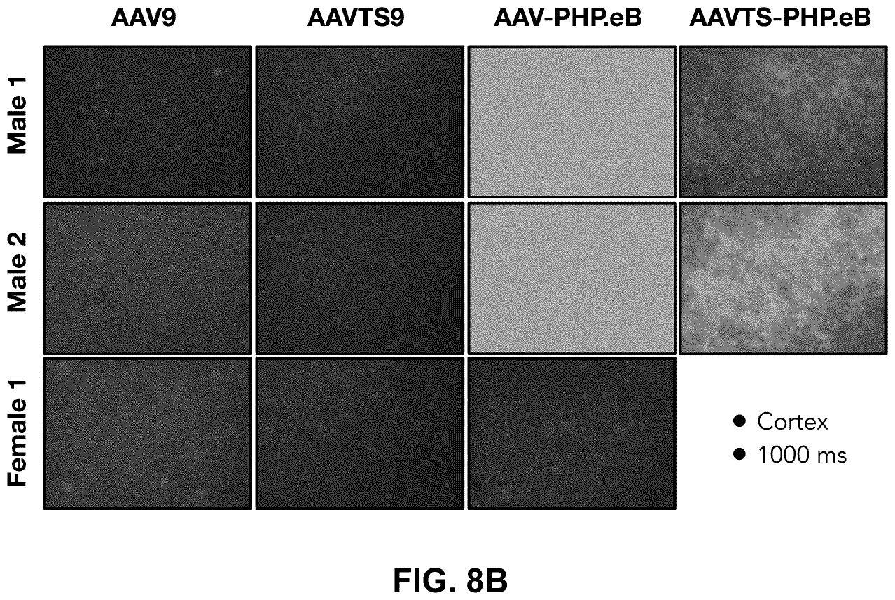

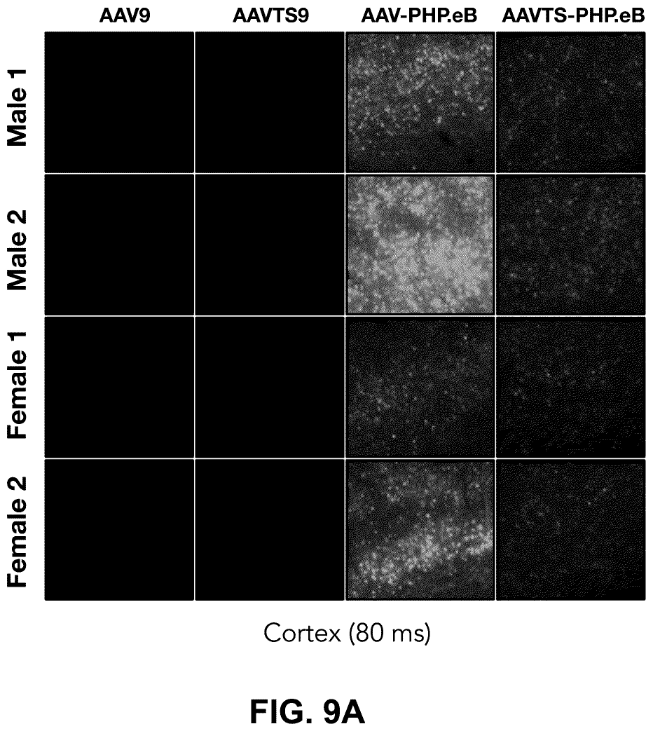

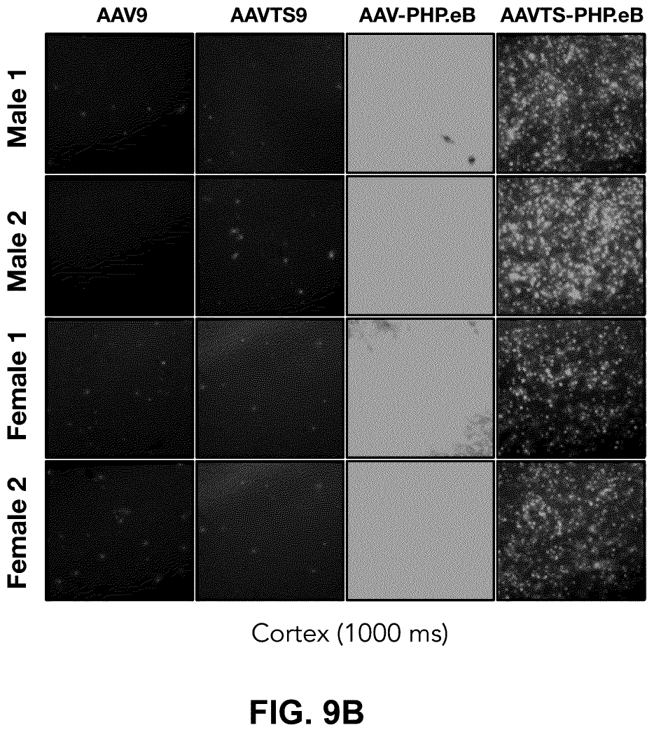

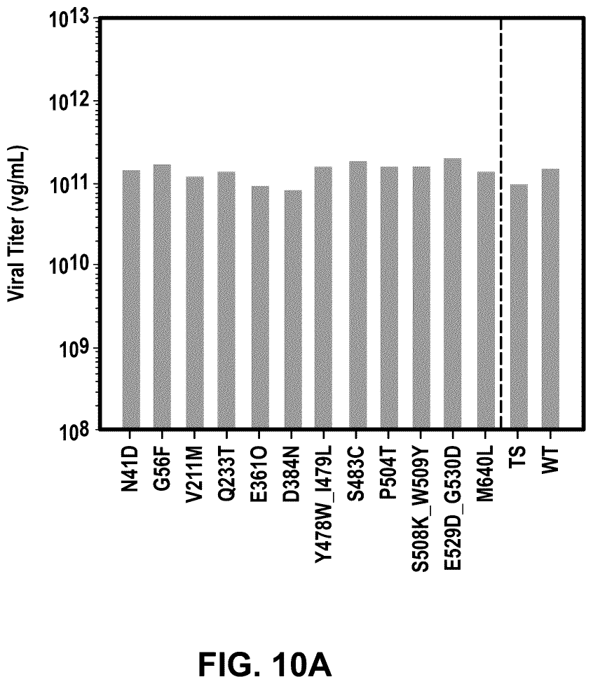

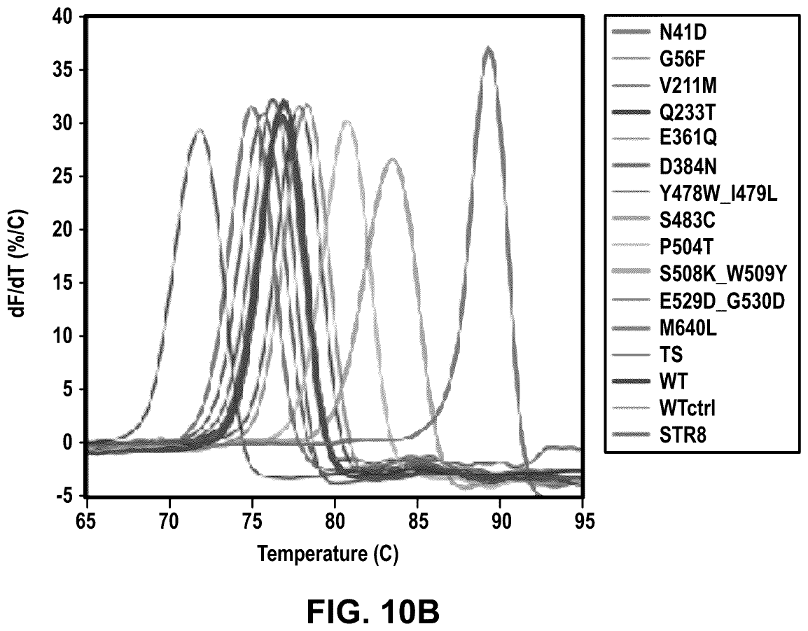

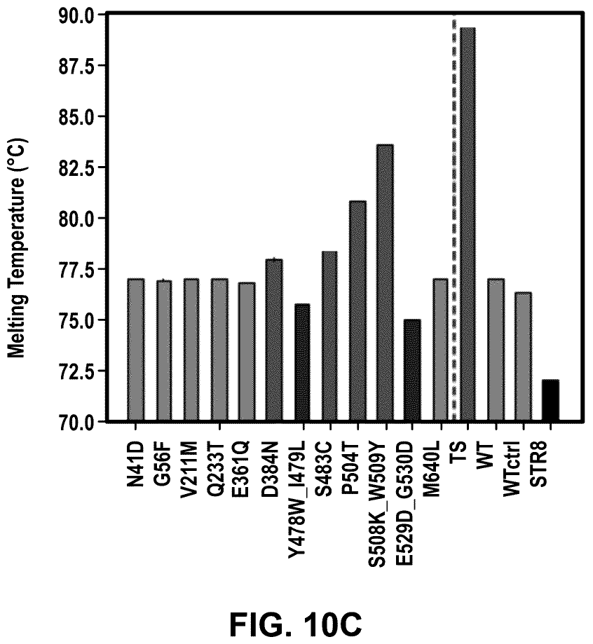

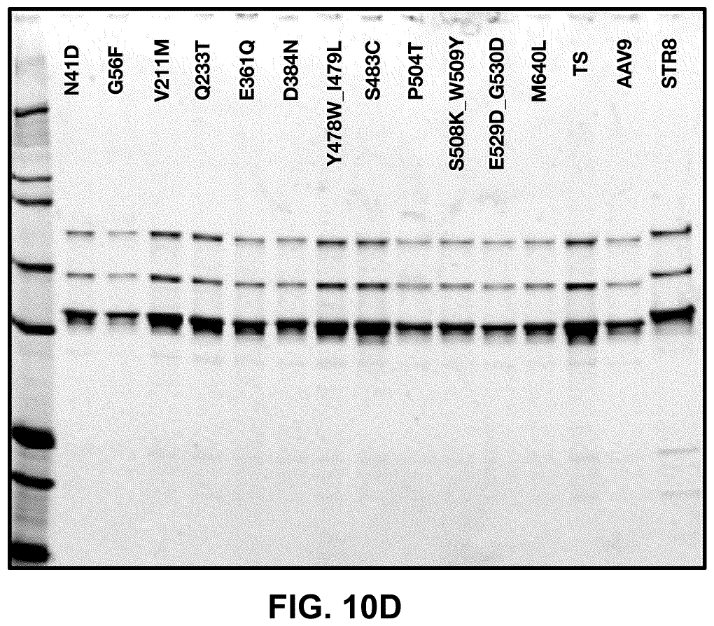

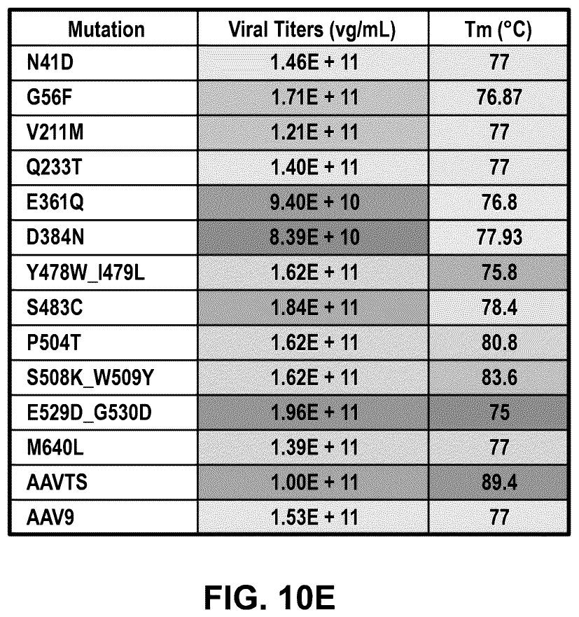

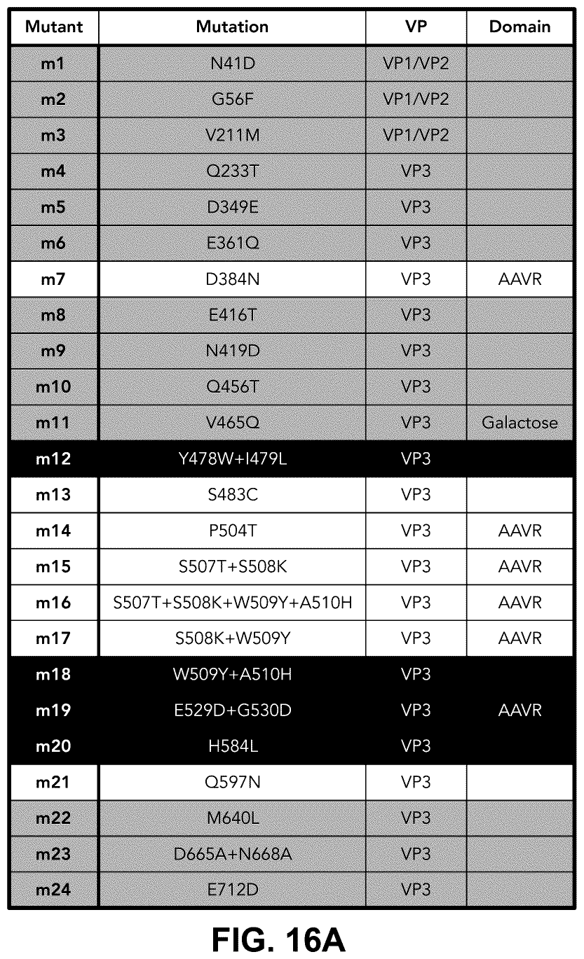

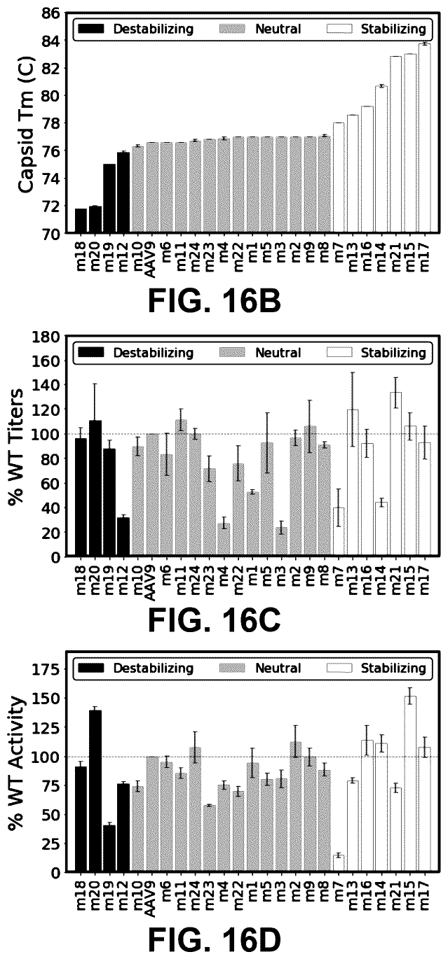

To see whether mutating the target residues “back-to-consensus” could increase the thermal stability of AAV9, AAVTS9 was cloned, produced and purified, an AAV9 mutant harboring 14 of the 15 previously identified mutations (FIG. 3A). Interestingly, DSF analysis of AAVTS9 revealed a 12.8° C. increase in thermal stability for this serotype (Tm=89.4° C.), relatively to AAV9 (Tm=76.6° C.) (FIG. 3B-3C) A total of 24 capsid mutants were cloned, produced, purified, and subjected to DSF (FIG. 16A). The Applicants found that 4 mutants were destabilized by 0.73-4.8° C., 7 mutants were stabilized by 1.4-7.13° C., and 13 mutants were not stabilized nor destabilized relatively to AAV9 (FIG. 16B). Analysis of the mutant amino acid composition suggested that 7 of the 26 consensus mutations were stabilizing to the AAV9 capsid: D384N, S483C, P504T, S507T, S508K, W509Y, and Q597N. In addition, analysis of production titers by ddPCR, in producing cell crude lysates, indicated that all mutants produced within 4.2-fold of AAV9 levels (FIG. 16C). Last, all 24 mutants were shown to retain their ability to transduce human cells, as measured by in vitro transduction assays in HEK293T cells (FIG. 16D).

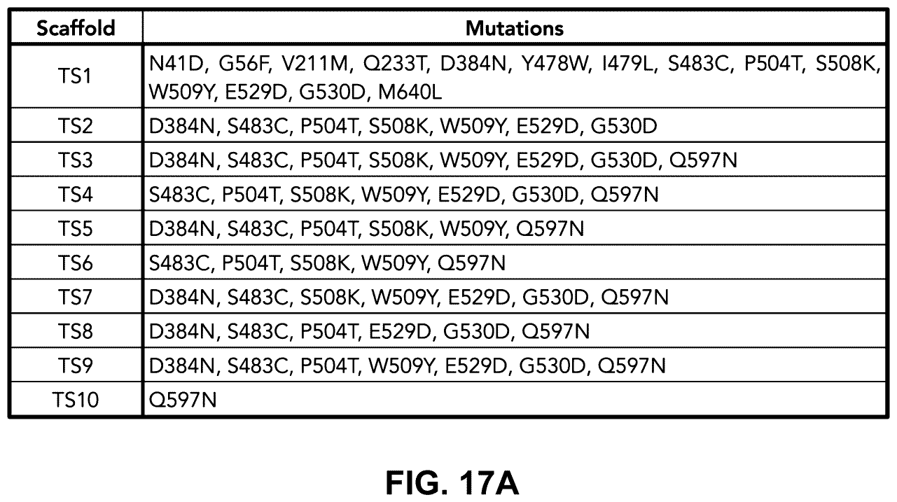

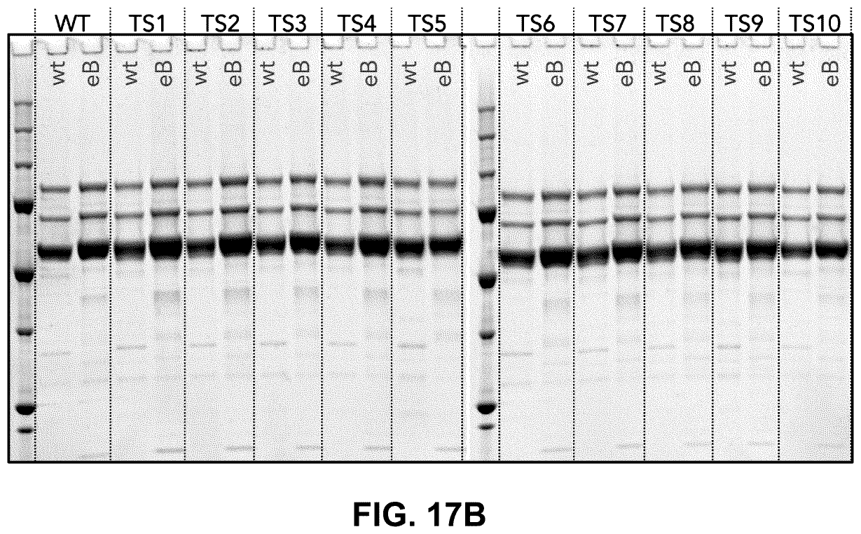

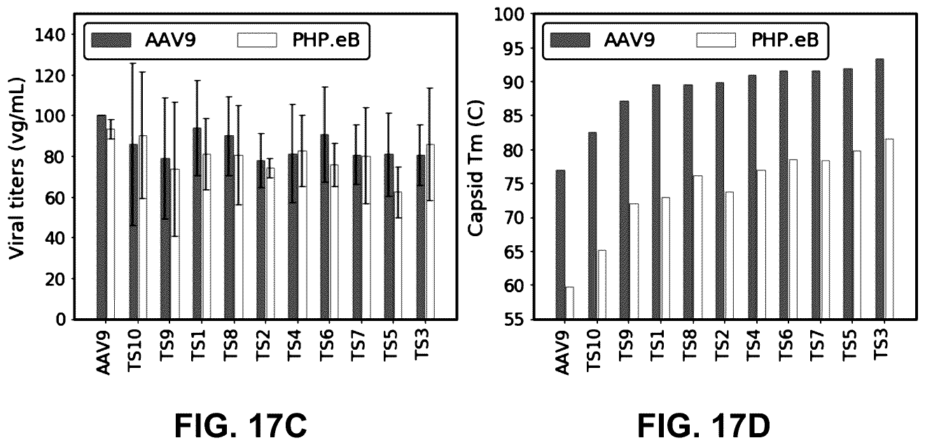

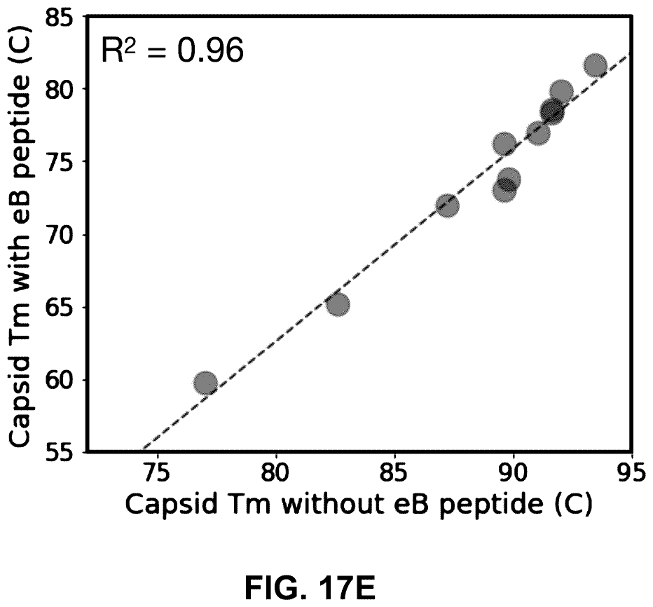

Next, Applicants sought to combine multiple consensus mutations, to increase AAV9 capsid Tm, as well as to investigate the possibility of stabilizing engineered AAV variants with low Tm, such as AAV-PHP.eB. Ten AAV9 mutants, referred to as thermal stable (TS1-TS10) (FIG. 17A), were produced individually and used to package an AAV dual reporter genome (AAV-CAG-GFP-P2A-Luciferase-WPRE-SV40), in the presence or absence of the PHP.eB peptide in VR VIII. Following purification with POROS AAV9, the 22 resulting AAV preparations were subjected to SDS-PAGE, ddPCR and DSF. All preparations had comparable purity profiles, VP1:VP2:VP3 stoichiometry, and VP/VG levels (FIG. 17B). In addition, the viral titers measured in crude cell lysates (n=3 transfection per variant) were within 2-fold of those measured for AAV9 and AAV-PHP.eB, suggesting that the selected combination of mutations were not detrimental to the production of AAV9 and AAV-PHP.eB (FIG. 17C). The selected combinations of mutations were also shown to stabilize the AAV9 capsid, with a Tm increase ranging from 5.6° C. to 16.4° C. (FIG. 17D). The DSF analysis of AAV-PHP.eB derived mutants also indicated that the selected combinations of mutations could stabilize AAV-PHP.eB to a greater extent, with a Tm increase ranging from 5.4° C. to 21.8° C. (FIG. 17E). A high correlation could be observed between the AAV9 and AAV-PHP.eB mutant capsid melting temperatures, suggesting that the consensus mutations and PHP.eB peptide insertion impacted the AAV9 capsid Tm in an independent fashion.

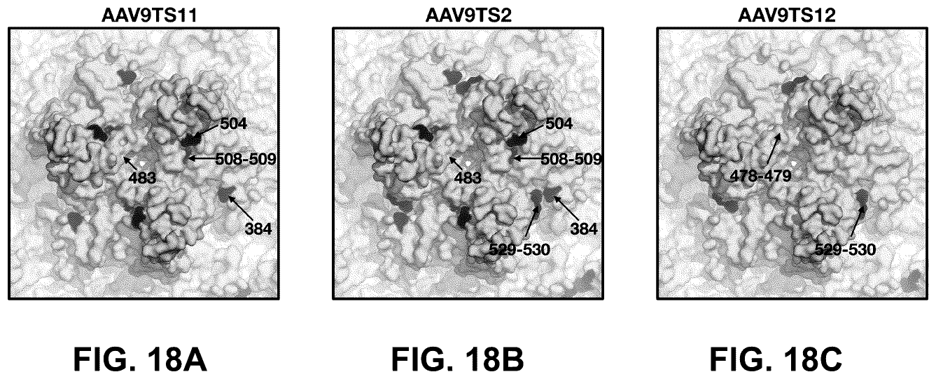

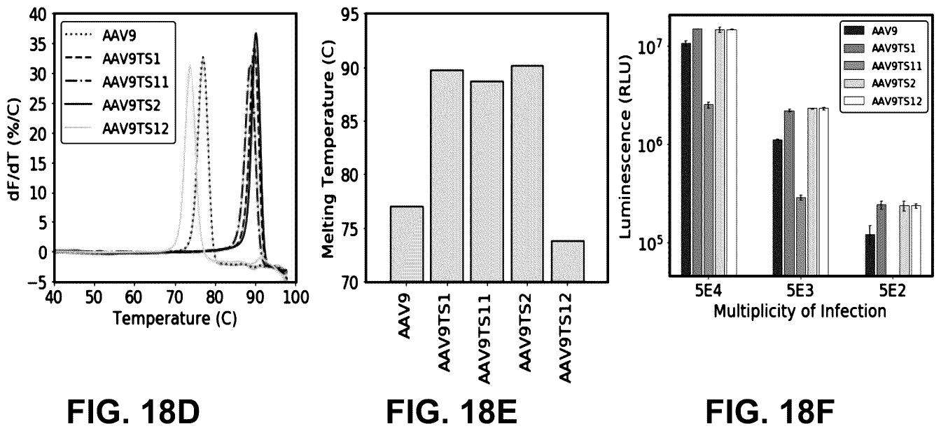

Applicants then investigated the epistatic interactions between stabilizing and destabilizing residues. Analysis of AAV9 crystal structure revealed that the stabilizing residue D384E was in close proximity of the destabilizing residues E529 and G530. To study the impact of this interaction on the thermal stability and functions of AAV9, the Applicants produced, purified, and characterized three AAV9 mutants, packaging an AAV-CAG-GFP-P2A-Luciferase-WPRE-SV40 genome. The first variant, AAV9TS1, was generated via introduction of the stabilizing mutations D384N, S483C, P504T, S508K and W509Y into AAV9 (FIG. 18A). The second mutant, AAV9TS2, was generated by adding the destabilizing mutations E529D and G530D to the first mutant (FIG. 18B). The third and last mutant, AAV9TS12, was generated via introduction of the destabilizing mutations Y478W, I479L, E529D and G530D into AAV9 (FIG. 18C). As expected, AAV9TS11 was stabilized relatively to AAV9, with a Tm reaching 88.7° C. (FIG. 18D-18E). This change in capsid thermal stability correlated with a 10-fold decrease in in vitro transduction compared to AAV9 (FIG. 18F). Surprisingly, adding destabilizing mutations E529D and G530D to AAV9TS11 further stabilized the viral capsid (Tm=89.8° C.) (FIG. 18D-18E, AAV9TS2) and restored in vitro transduction to AAV9 levels (FIG. 18F, AAV9TS2). These data suggest that residues 384, 529 and 530 are under coevolutionary pressure, and demonstrate that in some cases, the stabilizing effect of mutations may only be revealed when the consensus mutations are co-occurring. Last, AAV9TS12 was destabilized relatively to AAV9 (DeltaTm=3.1° C.) (FIG. 18D-18E) yet remained as effective as AAV9 at transducing HEK293T cells (FIG. 18F). Analysis of the AAV9TS12 capsid also showed that destabilizing mutations identified via consensus mutagenesis could be combined to generate functional, destabilized AAV scaffolds.

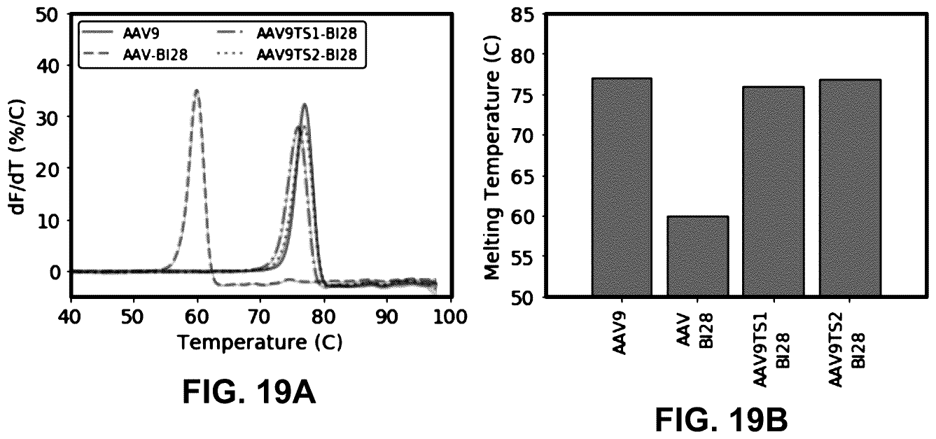

To see whether other engineered AAV variants with low Tm could be stabilized by consensus mutagenesis, the Applicants produced and purified AAV9, AAV-BI28, AAV9TS1-BI28 and AAV9TS2-BI28, packaging the dual reporter genome AAV-CAG-NLS-mScarlet-P2A-Luciferase-WPRE-SV40. The preparations were then subjected to DSF. As expected, insertion of the BI28 7-mer peptide in VR VIII of AAV9 strongly destabilized the viral capsid, by 17° C. (FIG. 19A-19B). In line with previous experiments, the TS1 and TS2 mutations (FIG. 18A) increased the capsid Tm of AAV-BI28 by 16° C. and 16.9° C., respectively (FIG. 19A-19B). This indicated that the Applicants' consensus mutagenesis approach could be used to stabilize multiple engineered AAV variants with low Tm.

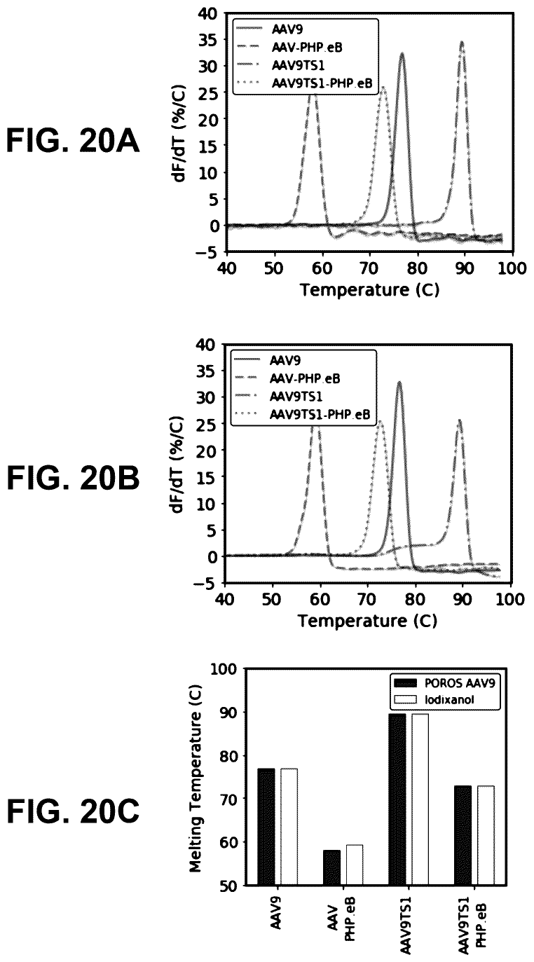

To investigate the impact of the packaged transgene and purification method on capsid stabilization via consensus mutagenesis, Applicants generated AAV9, AAV9TS1, AAV-PHP.eB and AAV9TS1-PHP.eB preparations, using two different transgene cassettes and two different purification methods. Four AAV preparations packaging the single reporter transgene CAG-NLS-GFP-WPRE were purified by iodixanol gradient ultracentrifugation (IDX), whereas four AAV preparations packaging the dual reporter CAG-GFP-P2A-Luciferase-WPRE-SV40 were purified by capture affinity followed by IDX. Results suggested that the stabilization of AAV9 and AAV-PHP.eB capsids, observed upon introduction of the TS1 mutations, was independent of the packaged transgene and purification method (FIG. 20A-20C).

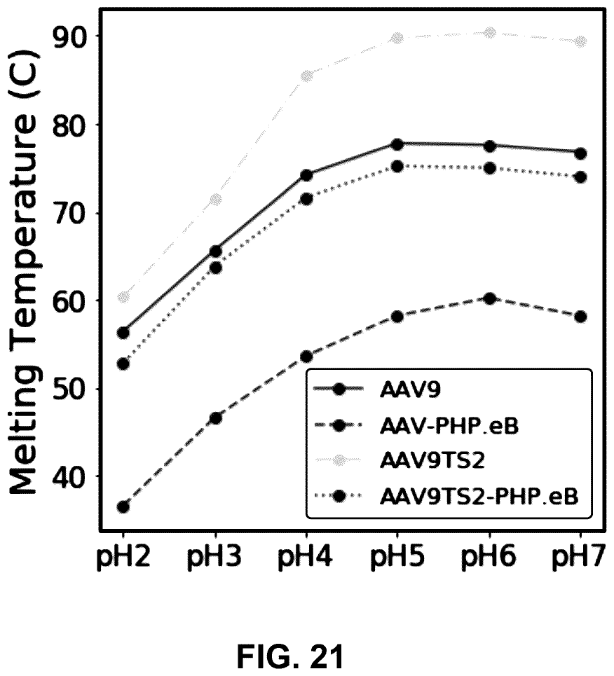

Applicants then sought to investigate whether the selected consensus mutations could stabilize the AAV9 capsid at pH 2-7. Assessing stability at pHs down to 2.5-3 is important to insure that capsids are stable under conditions similar to those used during AAV elution off of affinity columns commonly used for purification (Mietzsch, Smith et al, 2020). AAV9, AAV-PHP.eB, AAV9TS2 and AAV9TS2-PHP.eB, carrying a dual reporter transgene (CAG-GFP-P2A-Luciferase-WPRE-SV40) were diluted 1:10 in 0.1M sodium acetate, at pH 2-7, and subjected to DSF (FIG. 21). In agreement with previous studies (Pacouret et al, 2017), AAV9 Tm remained constant at pH7, pH6 and pH5, and decreased between pH4, pH3 and pH2. Interestingly, the delta in capsid Tm observed between AAV9 and AAV-PHP.eB remained consistent at all tested pH. In addition, the TS2 mutations were more stabilizing to AAV9 at pH 4-7 (DeltaTm=11.4-12.6° C.) than at pH 2-3 (DeltaTm=4.0-6.0° C.). Last, TS2 mutations stabilized AAV-PHP.eB in a pH-independent manner, with a difference in capsid Tm of 15.8-18° C. at pH2-7.

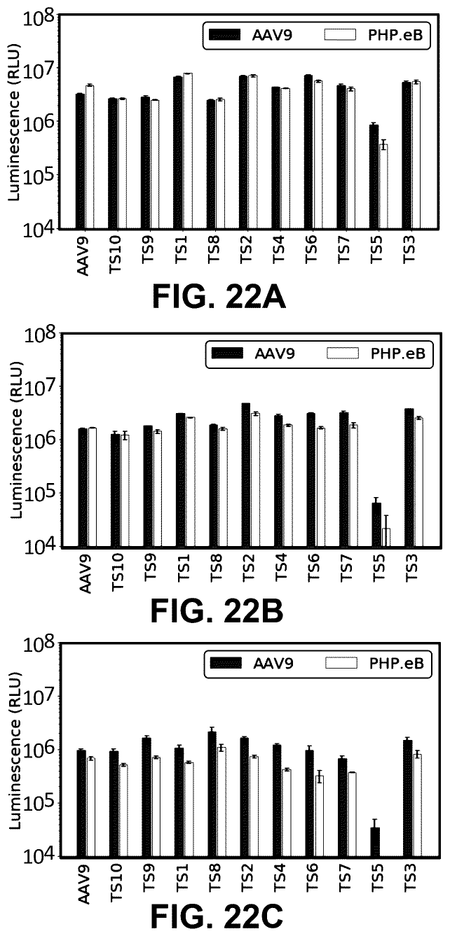

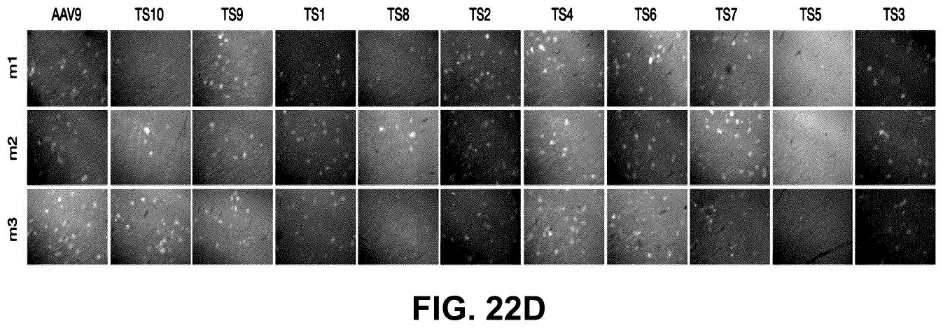

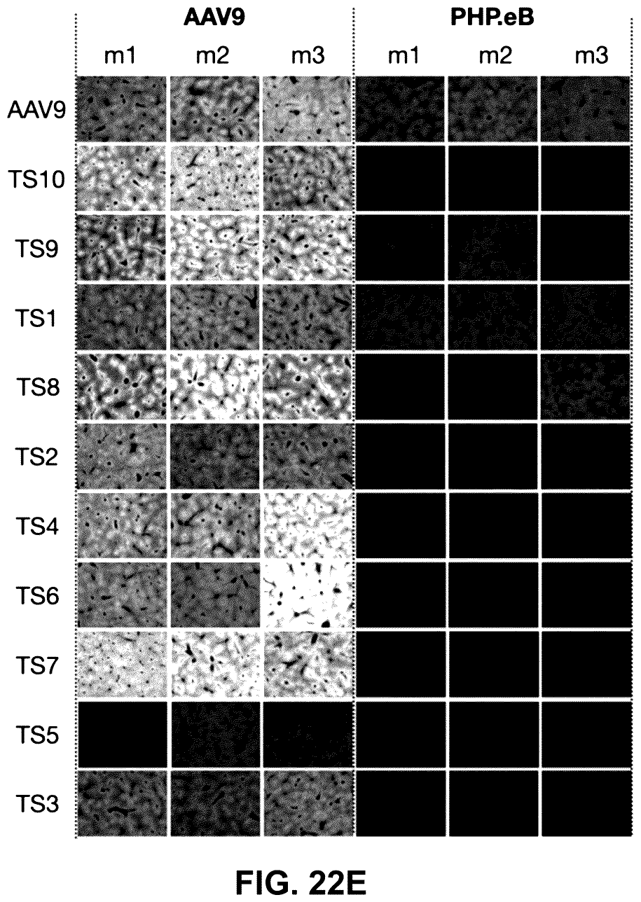

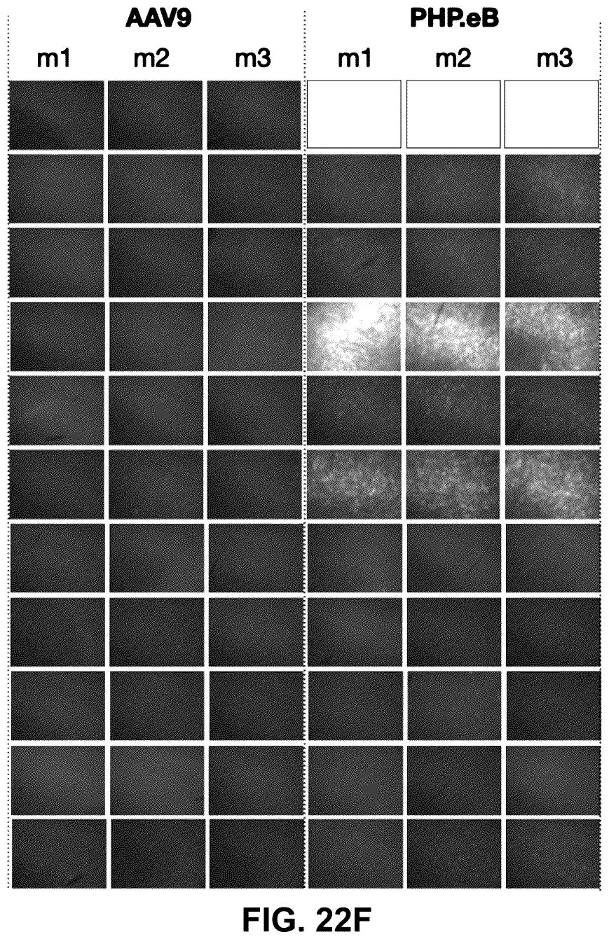

After these encouraging results, Applicants investigated the function of stabilized AAV capsids, through in vitro and in vivo transduction assays. HEK293T, hCMEC, and CHO cells were transduced with AAV9, AAV9TS1 to AAV9TS10, AAV-PHP.eB and AAV9TS1-PHP.eB to AAV9TS10-PHP.eB vectors, packaging a dual reporter transgene (GFP-P2A-luciferase), at 5E4, 6.7E4 and 5E4 vector genomes per cell (vg/cell), respectively. 48 h later, luminescence levels of lysed producing cells were measured using a spectrophotometer (FIG. 22A-22C). Results suggested that the combinations of consensus mutations tested were not detrimental to the in vitro transduction efficiency of AAV9 and AAV-PHP.eB. With the exception of AAV9TS5, every stabilized capsid was shown to transduce all three cell lines within 2-fold of the parental capsids AAV9 and AAV-PHP.eB levels (FIG. 22A-22C).

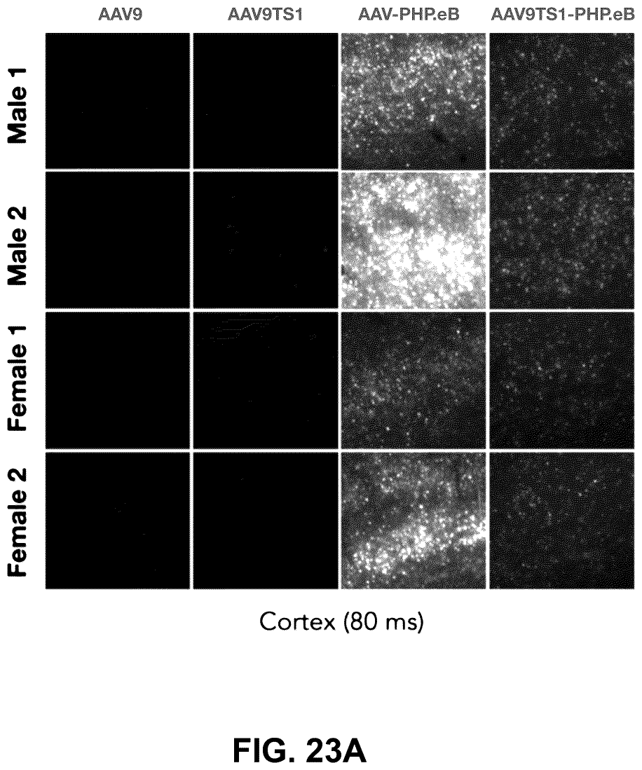

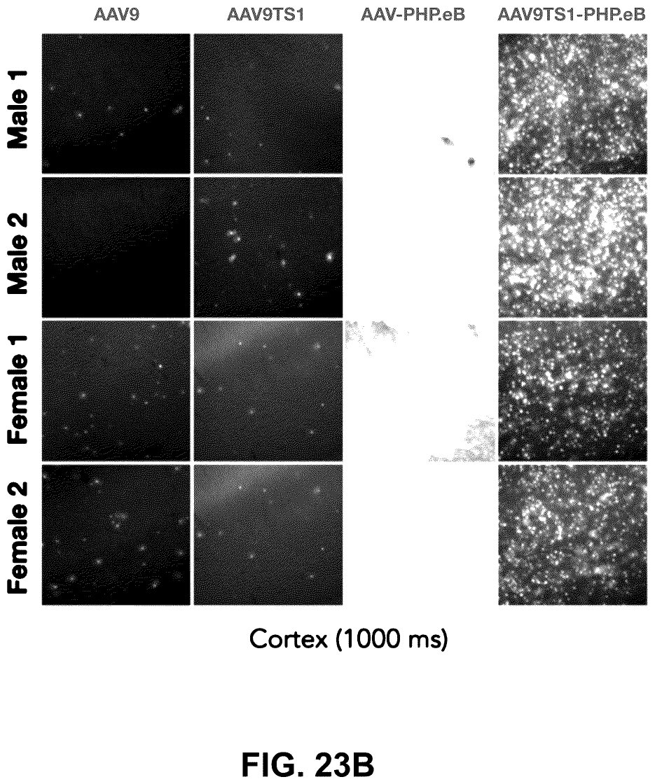

To evaluate the in vivo transduction profiles of stabilized capsid variants, Applicants first administered AAV9, AAV9TS1, AAV-PHP.eB and AAV9TS1-PHP.eB packaging the AAV-CAG-NLS-GFP-WPRE genome into mice, at a dose of 1e11 vg per animal (n=4). Four weeks post-administration, brain tissues were harvested, sliced, and imaged by fluorescence microscopy. Analysis of the brain sections of injected animals revealed that AAV9TS1 maintained a BBB-crossing phenotype upon systemic delivery, transducing cells morphologically identifiable as astrocytes and neurons at levels comparable to AAV9 (FIG. 23A-23B). AAV9TS1-PHP.eB was also capable of crossing the BBB, with transduction levels exceeding those of AAV9 and AAV9TS1. However, AAV9TS1-PHP.eB provided less efficient CNS transduction than AAV-PHP.eB.

These results were confirmed by injecting a second cohort of mice, at the same dose, with AAV9, AAV9TS1 to AAV9TS10, AAV-PHP.eB and AAV9TS1-PHP.eB to AAV9TS10-PHP.eB, packaging the dual reporter CAG-GFP-P2A-Luciferase-WPRE-SV40. Three weeks post-administration, liver and brain tissues were harvested, sliced, and imaged by fluorescence microscopy. All stabilized AAVs transduced the brain and liver in vivo, to the exception of AAV9-TS5 and AAV9TS5-PHP.eB (FIG. 22D-22F), in line with the in vitro data (FIG. 22A-22C). In the absence of the PHP.eB peptide, all other stabilized mutants transduced the mouse liver with various efficiency, with AAV9TS3 and AAV9TS7 being the least and most efficient, respectively (FIG. 22E). In the presence of the PHP.eB peptide, all stabilized AAVs appeared to be detargeted from the liver relative to the unmodified AAV-PHP.eB capsid (FIG. 22E). Analysis of sagittal brain sections suggested that AAV9TS1-4 and AAV9TS6-10 all crossed the BBB at levels comparable to AAV9 (FIG. 22D). AAV9TS1-PHP.eB to AAV9TS4-PHP.eB and AAV9TS6-PHP.eB to AAV9TS10-PHP.eB also provided CNS transduction, with AAV9TS1-PHP.eB being the most efficient, followed by AAV9TS2-PHP.eB, AAV9TS8-PHP.eB, AAV9TS10-PHP.eB and AAV9TS9-PHP.eB. Nevertheless, these stabilized variants transduced the brain at lower levels than their parental capsid AAV-PHP.eB (FIG. 22F), confirming that the PHP.eB peptide was not optimized for these new stabilized capsid scaffolds.

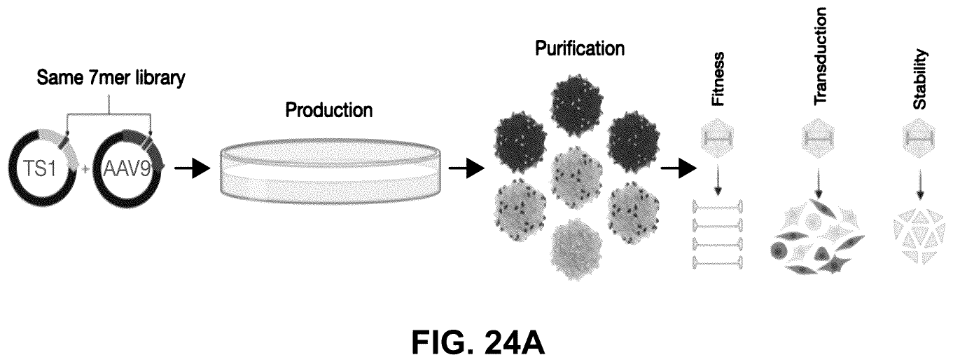

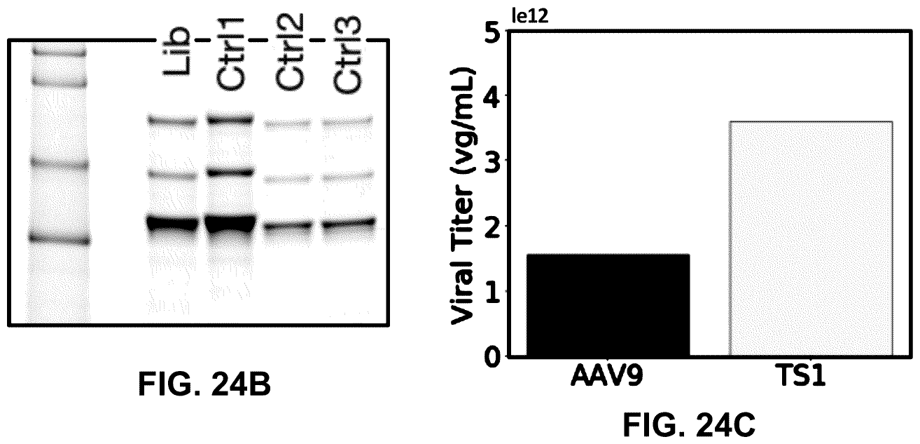

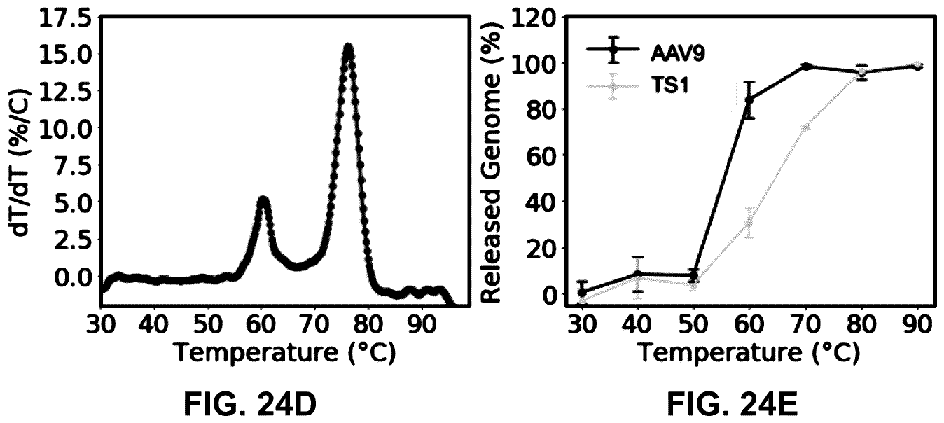

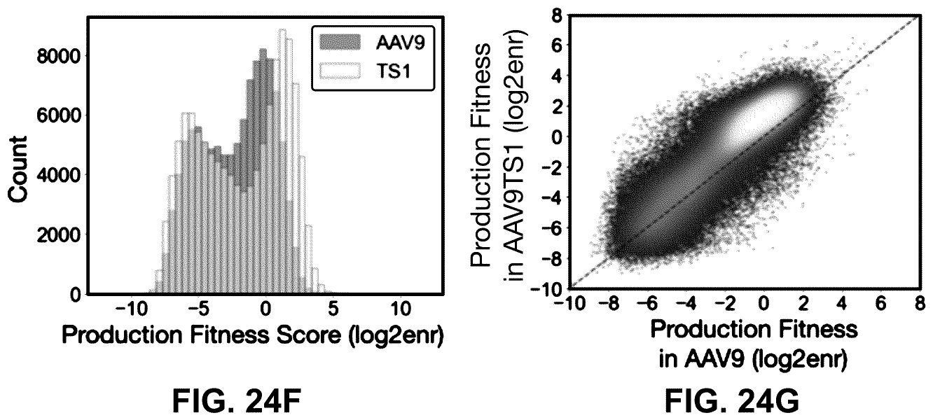

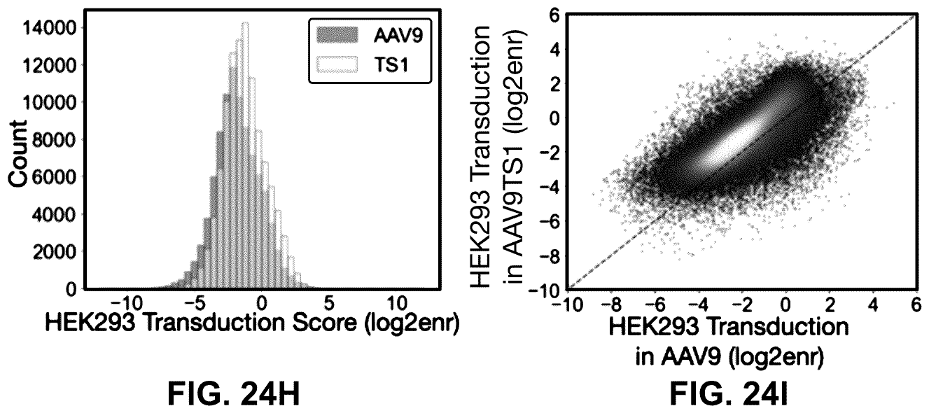

Finally, AAV9TS1 tolerance to 7-mer library peptide insertion was compared to that of AAV9, using a previously characterized 7-mer synthetic oligo pool library that randomly samples the 7-mer amino acid sequence space (Eid et al., Systematic multi-trait AAV capsid engineering for efficient gene delivery, bioRxiv, 2022, doi: 10.1101/2022.12.22.521680) (FIG. 24A). This library was cloned into AAV9 and AAV9TS1 expression plasmids (AAV-RNA Express), at amino acid position 588 (VP1 numbering). Both plasmid libraries were pooled at a 1:1 ratio, prior to transfection into HEK293T cells for AAV library production and purification by iodixanol gradient ultracentrifugation. SDS-PAGE analysis showed that the resulting library was pure, with the expected 1:1:10 VP stoichiometry (FIG. 24B). Using a cap-specific duplex ddPCR assay, both AAV9TS1 and AAV9 library variant genomes could be detected, at a ratio of 3:1 (FIG. 24C), indicating that AAV9TS1 variants produced at higher levels than their AAV9 counterparts. Analysis of the library by DSF also suggested the presence of AAV9 and AAV9TS1 library capsid variants, with mean melting temperatures of 60.1° C. and 76.2° C., respectively (FIG. 24D). This profile indicated that, like the AAV-PHP.eB and BI28 7-mers, 7-mer variant capsids, regardless of sequence are generally stabilized by the TS mutations. To measure whether the TS1 library is also more resistant to heat mediated genome release, the capsid library was incubated at various temperatures and subjected to digestion with Turbonuclease followed by cap-specific duplex ddPCR to measure the Turbonuclease-resistant genomes (FIG. 24E). In line with the DSF data, the results suggested that AAV9TS1 variants released their genomes at a higher temperature than AAV9 variants. For instance, following incubation of the capsid library for 5 min at 60° C., 84% of AAV9 variants had released their genomes, as compared to only 31% for AAV9TS1 variants (FIG. 24E).

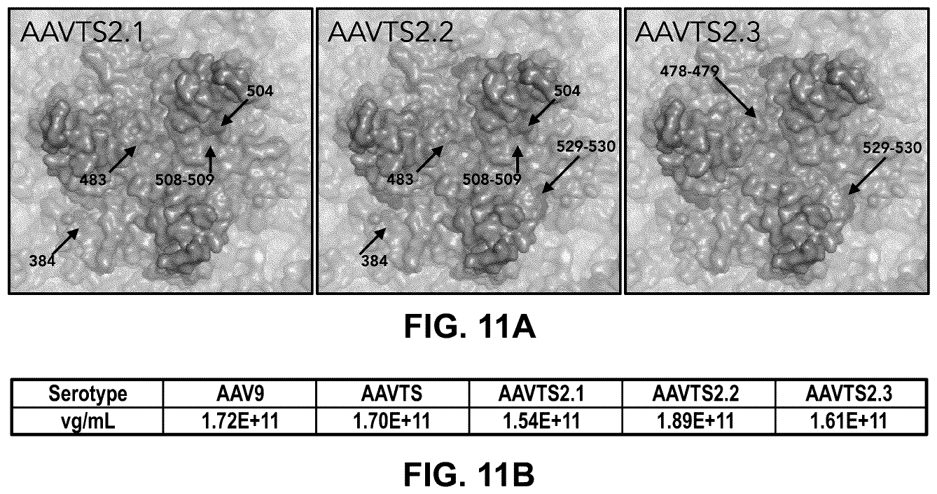

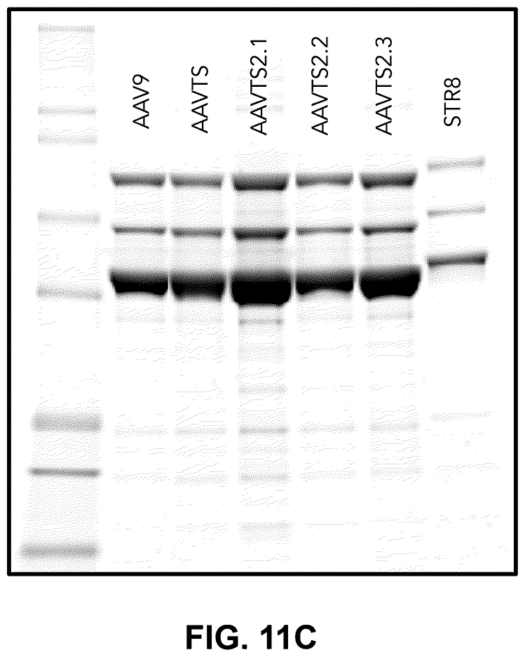

In addition, NGS data showed that the distributions of variant log 2 enrichment production fitness scores were right-shifted and narrower for AAVTS9 in comparison to AAV9, indicating an increase in fitness for a fraction of variants in the stabilized library (FIG. 24F). In support of this conclusion, a larger fraction of the input variants in the AAV9TS91 scaffold was detected than in the AAV9 scaffold following purification (79% vs 72%). The variant log 2 enrichment production fitness scores measured in the AAV9 and AAV9TS1 scaffolds showed a high degree of correlation, indicating that the TS1 stabilizing mutations are not detrimental to the fitness of a broad range of AAV9-derived variants with 7-mer peptides inserted in VR VIII (FIG. 11G).

Last, the Applicants subjected the library to a high throughput in vitro transduction screen in HEK293T cells (FIG. 24H-24I). The distribution of variant log 2 enrichment transduction scores was right shifted for AAV9TS1 relatively to AAV9, suggesting an increase in transduction efficiency for a fraction of variants in the stabilized library (FIG. 24H). In support of this conclusion, a larger fraction of the input variants in the AAV9TS1 scaffold was detected than in the AAV9 scaffold following purification (71% vs 61%). The variant log 2 enrichment transduction scores measured in the AAV9 and AAV9TS1 scaffolds also showed a high degree of correlation, indicating that the TS1 stabilizing mutations are not detrimental to the function of a broad range of 7-mer peptide variants (FIG. 24I).

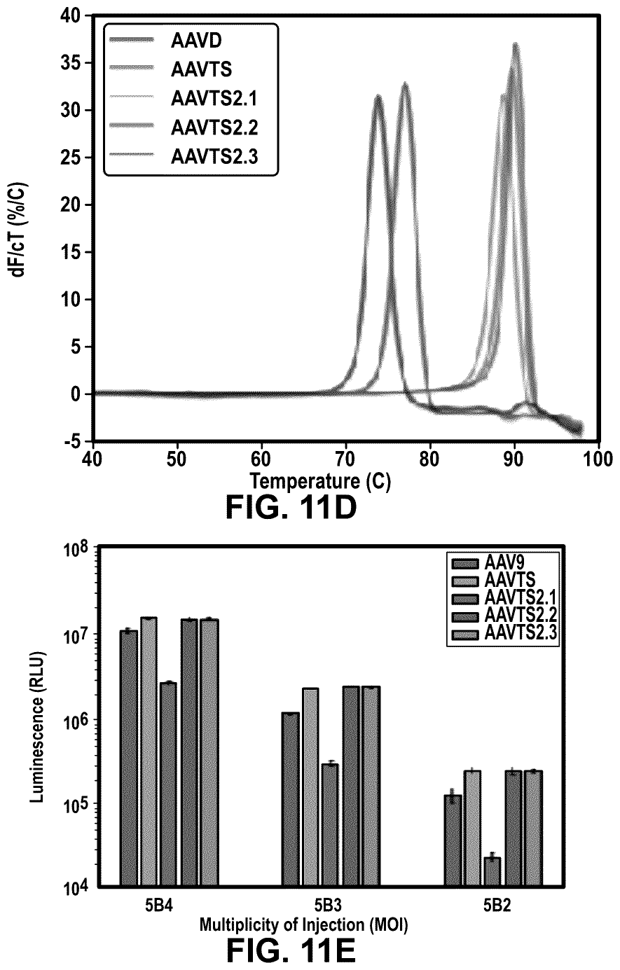

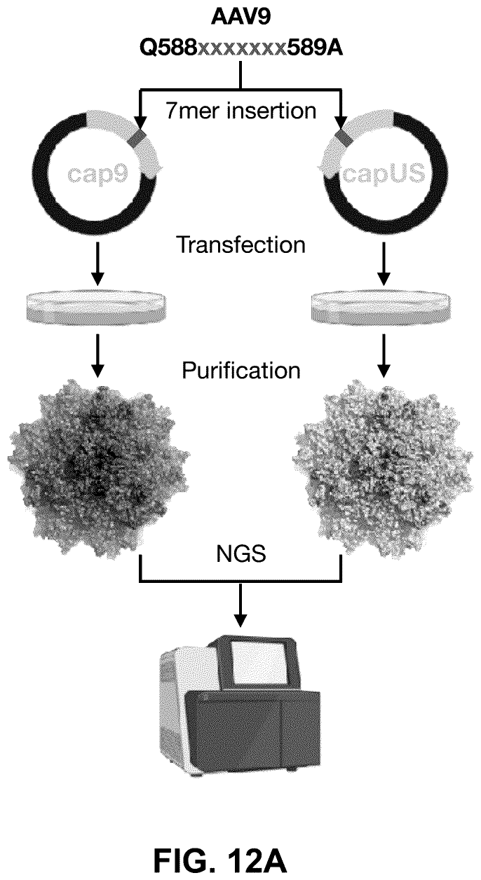

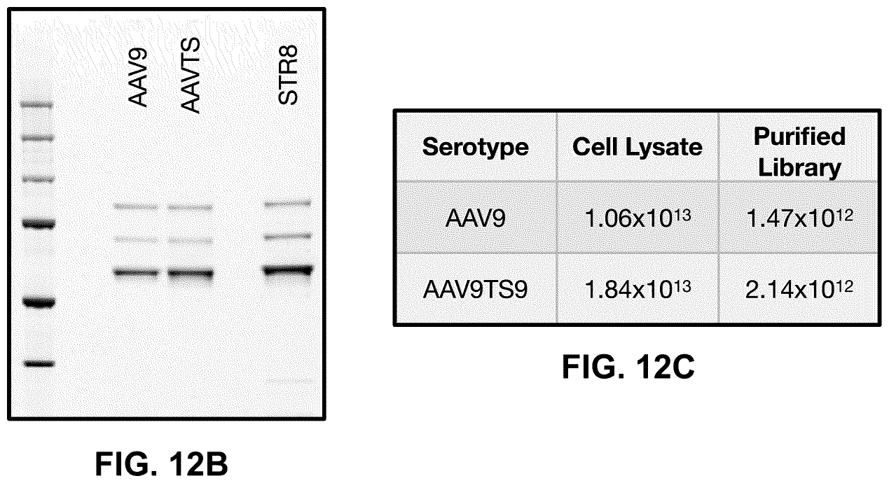

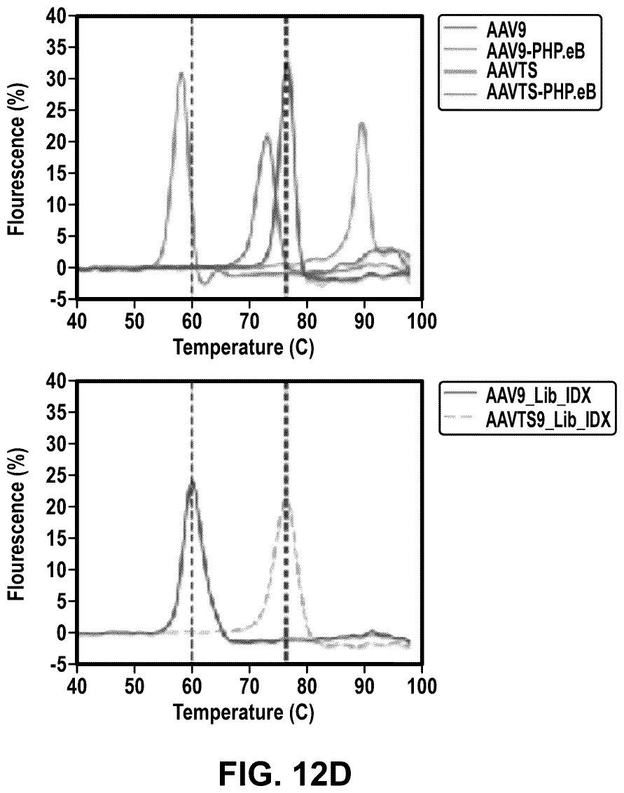

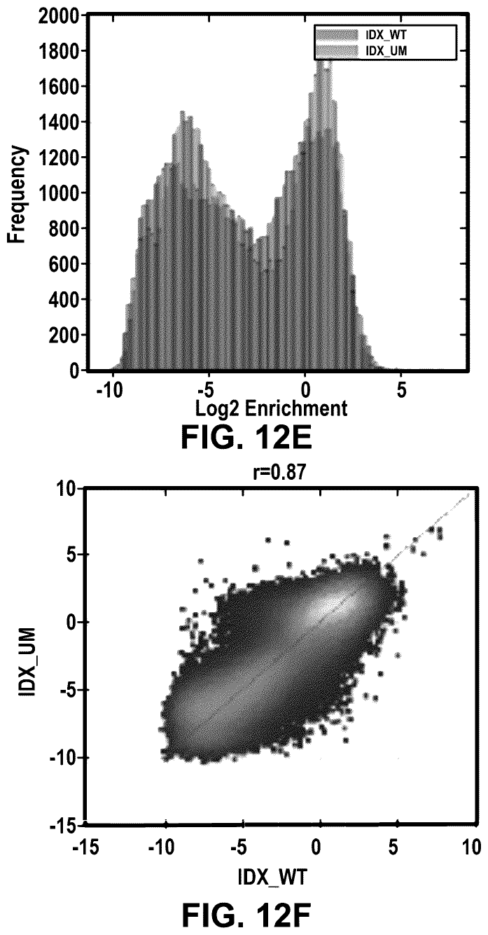

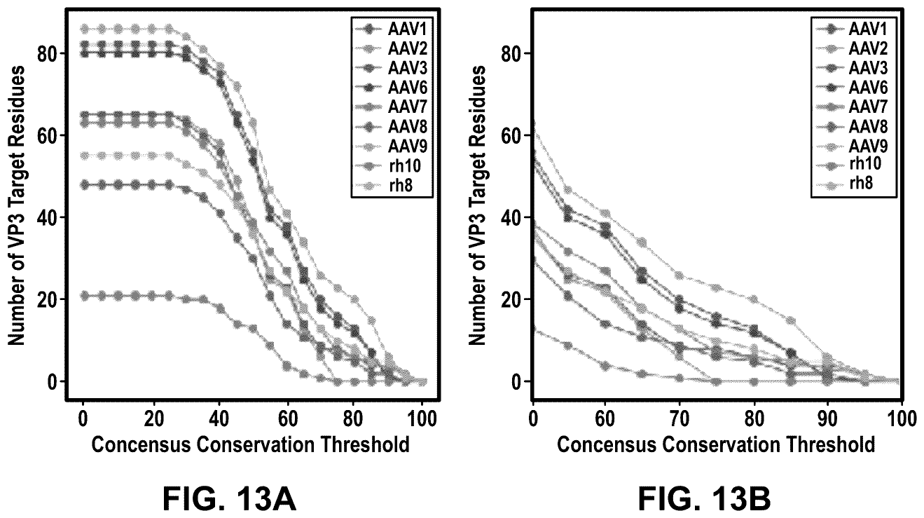

Last, the study was extended to other naturally occurring serotypes. Using the multiple sequence alignment presented in FIG. 2A, singletons residues in the VP3 sequences of AAV1, AAV2, AAV3B, AAV6, AAV7, AAV8, AAVrh8 and AAVrh10 (FIG. 12A-12B) were identified.

### Discussion

The measure of the capsid Tm of 153 variants engineered from AAV9 revealed that mutations introduced during directed evolution tended to destabilize the AAV capsid. Based on this observation, the Applicants hypothesized that (1) directed evolution of the AAV capsid may be constrained by its thermal stability, and that (2) stabilizing mutations may allow the AAV capsid to tolerate further destabilizing mutations, leading to enhanced functions.

Analysis of the VP1 sequences of 75 AAV homologs led to the discovery of 7 stabilizing mutations and 7 destabilizing mutations. The Applicants further demonstrated, through the characterization of AAV9TS1-10, that these mutations could be combined to modulate the AAV capsid Tm (DeltaTm=5.6° C. to 16.4°), without compromising AAV9 production, in vitro transduction, and BBB-crossing phenotypes. The Applicants later showed that these mutations could also stabilize engineered capsid variants with low Tm, such as AAV-PHP.eB and AAV-BI28, and were compatible with 7-mer peptide library insertion in VR-VIII for directed evolution studies.

In line with Bloom et al., the extra stability provided by TS mutations may confer additional mutational robustness to the AAV9 capsid, enabling the Applicants to combine a higher number of capsid modifications beneficial to manufacturability, cross-species BBB penetration and immune evasion. These results show that it can be beneficial to use a highly stabilized capsid scaffold, e.g., AAVTS2, as a starting point for directed evolution. In this case, the extra 13° C. in thermal stability, relatively to AAV9, may counterbalance the decrease in capsid Tm due to 7-mer peptide insertion in VR VIII, allowing for the introduction of additional destabilizing mutations increasing tissue specificity and antibody resistance. However, in some use cases, a high increase in capsid thermal stability may also correlate with an increase in conformational rigidity, which may ultimately lower the mutational tolerance of the capsid structure (Strobel et al., 2022) or reduce specific functional attributes (e.g., CNS transduction by AAV-PHP.eB after intravenous administration). Therefore, other strategies could be envisioned such as spiking AAV capsid libraries, e.g., 7-mer peptide libraries, with individual stabilizing mutations (Tokoriki et al, 2009), hence providing additional conformations associated with marginal increases in thermal stability. The identification of a panel of individual and combinatorial (TS1-10) mutations facilitates this approach. This could result in an increase in capsid tolerance to the highly destabilizing peptides linked to novel functions, without highly rigidifying the capsid structure.

Alternatively, in other use cases, the discovery of destabilizing residues may also be useful for the development of capsids with novel functions, such as broad CNS transduction. These mutations may indeed provide additional flexibility to the AAV capsid, allowing surface exposed loops to adopt novel conformations favoring the interactions with non-native attachment factors and receptors (Strobel et al, 2022).

### Methods

### Identification of Target Residues for Consensus Mutagenesis

A multiple sequence alignment of the VP1 capsid protein sequences of 75 AAV serotypes (Zinn et al., In silico reconstruction of the viral evolutionary lineage yields a potent gene therapy vector, Cell Reports, 2015 12(6) 1056-1068) was generated using the T-coffee multiple alignment program (Notredame et al., T-Coffee: A novel method for fast and accurate multiple sequence alignment, Journal of Molecular Biology, 2000, 302(1) 205-217). Residues with a conservation score above 75%, non-conserved between AAV9 and the consensus sequence, were selected for further analysis.

### AAV Library Cloning

The RNA expression system for the selection of functional AAV capsids was used as previously described (Krolak et al., A high-efficiency AAV for endothelial cell transduction throughout the central nervous system, Nature Cardiovascular Research, 2022, 1(4) 389-400) with a modification to include a Woodchuck Hepatitis Virus (WHV) Posttranscriptional Regulatory Element (WPRE) between the restriction enzyme site SalI and HindIII. The wild type AAV9 and AAV9TS1 capsid gene sequences were synthesized (GenScript) with nucleotide changes at S448 (TCA to TCT, silent mutation), K449R (AAG to AGA), and G594 (GGC to GGT, silent mutation) to introduce XbaI and AgeI restriction enzyme recognition sites for library fragment cloning.

To assemble the AAV plasmid library, pUC57-wtAAV9-X/A and pUC57-AAV9TS1-X/A plasmids were amplified for 10 cycles with a 150 k synthetic oligo pool randomly sampling the 7-mer amino acid sequence space (Supreprint, Agilent):

(SEQ ID NO: 27)
GTATTCCTTGGTTTTGAACCCAACCGGTCTGCGCCTGTGCTNNNNNNNNN
NNNNNNNNNNNNTGGGCACTCTGGTGGTTTG

and the Assembly-XbaI-F oligo (CACTCATCGACCAATACTTGTACTATCTCT (SEQ ID NO: 28)) forward primer, using Q5® High-Fidelity 2× Master Mix (NEB #M0492S) following the manufacturer's protocol. Then, the reaction was spiked with 0.5 μM of primer Assembly AgeI-R (GTATTCCTTGGTTTTGAACCCAACCG (SEQ ID NO: 29)) and amplified for an additional 25 cycles. The PCR product was purified using a Zymoclean DNA Gel Recovery kit (Zymo Research #D4007) following the manufacturer's protocol. The 7-mer NNK or oligo pool PCR products were assembled into the RNA expression plasmid as previously described (Deverman et al., Cre-dependent selection yields AAV variants for widespread gene transfer to the adult brain, Nature Biotechnology, 2016, 34(2) 204-209).

### AAV Production and Purification

Vector preparations from FIGS. 3A-3C and 16A-16D were produced by triple transfection in HEK293T/17 cells (ATCC, CRL-11268), using the transfection reagent Polyethylenimine (PEI) (Polysciences, 26008-50). Prior to transfection, HEK293 cells were seeded in 6-well plates at a density of 2 million cells per well. Cells were transfected with 4 ug plasmid, using a pHelper:pRepCap:pTransgene ratio of 2:1:1, and a PEI:DNA ratio of 1.375:1, in serum-free DMEM. 72 hours post-transfection, the cells and supernatant from each well (2.2 mL total) were collected in 5 mL Eppendorf, incubated with 0.1% Triton x100, 2 mM MgCl2 and 50 U/mL benzonase (Sigma, E1014-25KU), for 90 min at 37C, and centrifuged for 10 min at 10,000 rpm for clarification. The clarified lysates were incubated with 50 uL POROS AAV9 resin (Thermofisher, A27353), and rocked at 37C for 90 min. The mixes were loaded into detergent removal spin columns (Thermofisher, 87778), and supernatants were discarded using a vacuum manifold. POROS AAV9 beads were rinse 3 times with 5 mL PBS, using a vacuum manifold. AAVs were eluted in 15 mL Falcon tubes, with 2 mL elution buffer (0.1M Glycine, pH2.5) and neutralized with 500 uL 1M Tris, 0.5M NaCl, pH8. AAVs were buffer exchanged and concentrated in PBS (with calcium and magnesium) supplemented with 0,001% pluronic F68, using Amicon filters (Millipore), with a molecular weight cutoff (MWCO) of 100 kDa.

Vector preparations from FIGS. 17A-17E, 19A-19B, and 20A-20C were produced by triple transfection in HEK293T/17 cells (ATCC, CRL-11268), using Polyethylenimine (PEI). Prior to transfection, HEK293 cells were seeded in 15-cm dishes at a density of 20 million cells per dish. A total of three dishes were used for each vector preparation. Cells were transfected with 40 ug plasmid per 15-cm dish, using a pHelper:pRepCap:pTransgene ratio of 2:1:1, and a PEI:DNA ratio of 1.375:1, in serum-free DMEM. 72 hours post-transfection, the cells and supernatant from each prep (60 mL total) were collected in 125 mL shake flasks, incubated with 0.1% Triton x100, 2 mM MgCl2 and 50 U/mL benzonase (Sigma, E1014-25KU), for 90 min at 37C, and centrifuged for 10 min at 4000 rpm for clarification. The clarified lysates were incubated with 150 uL POROS AAV9 resin (Thermofisher, A27353), at 37C for 90 min, under agitation (200 rpm). The mixes were loaded into detergent removal spin columns (Thermofisher, 87778), and supernatants were discarded using a vacuum manifold. POROS AAV9 beads were rinse 3 times with 5 mL PBS, using a vacuum manifold. AAVs were eluted in 15 mL Falcon tubes, with 2 mL elution buffer (0.1M Glycine, pH2.5) and neutralized with 500 uL 1M Tris, 0.5M NaCl, pH8. AAVs were buffer exchanged and concentrated in PBS (with calcium and magnesium) supplemented with 0,001% pluronic F68, using Amicon filters (Millipore), with a molecular weight cutoff (MWCO) of 100 kDa.

The AAV libraries from FIG. 24A-24I were generated by triple transfection of HEK293T/17 cells (ATCC, CRL-11268) using polyethylenimine (PEI), purified by ultracentrifugation over iodixanol gradients, and titered as previously described (Deverman et al., 2016; Krolak et al., 2022).

### AAV Titration

Vector preparations or crude lysates was incubated with 1000 U/mL Turbonuclease (Sigma T4330-50KU) with 1×DNase I reaction buffer (NEB B0303S) at 37° C. for one hour. The endonuclease solution was inactivated with 0.5M, pH 8.0 EDTA at room temperature for 5 minutes and then at 70° C. for 10 minutes. AAV genomes were released by incubation with 100 g/mL Proteinase K (Qiagen, 19131) in 1M NaCl, 1% N-lauroylsarcosine, and in UltraPure DNase/RNase-Free water at 56° C. for 2 to 16 hours before heat inactivation at 95° C. for 10 minutes. The nuclease-resistant AAV genomes were diluted between 460-460,000× and 2 μL of the diluted samples were used as input in a ddPCR supermix for probes (Bio-Rad, 1863023) with 900 nM CAG-Forward primer, 900 nM CAG-Reverse primer, 900 nM ITR-Forward primer, 900 nM ITR-Reverse primer, 250 nM CAG-Probe-FAM and 250 nM ITR-Probe-HEX. Droplets were generated using a QX100 Droplet Generator, transferred to thermocycler, and cycled according to the manufacturer's protocol with an annealing/extension of 58° C. for 1 minute. Finally, droplets were read on a QX100 Droplet Digital System to determine titers. AAV library preparation viral genome levels were quantified using the same method, with AAV9-cap and TS1-cap specific primers and probes. The primers and probes used for titration are detailed below in Table 3 and Table 4, respectively:

TABLE 3

| | | Final |
|---|---|---|
| | | concen- |
| Primer | Sequence (5′→3′) | tration |
| CAG-FORWARD | TGTTCCCATAGTAACGCCAATAG | 900 nM |
| | (SEQ ID NO: 30) | |
| CAG-REVERSE | GTACTTGGCATATGATACACTTGATG | 900 nM |
| | (SEQ ID NO: 31) | |
| ITR-FORWARD | GGAACCCCTAGTGATGGAGTT | 900 nM |
| | (SEQ ID NO: 32) | |
| ITR-REVERSE | CGGCCTCAGTGAGCGA | 900 nM |
| | (SEQ ID NO: 33) | |
| Cap-AAV9- | CGGACCCAGCAACATGG | 900 nM |
| FORWARD | (SEQ ID NO: 34) | |
| Cap-TS1- | CGGACCCAGCAACATGG | 900 nM |
| FORWARD | (SEQ ID NO: 35) | |
| Cap-AAV9- | TGGTTGAGACACGTTGTTGTC | 900 nM |
| REVERSE | (SEQ ID NO: 36) | |
| Cap-TS1- | TGGTTGAGACACGTTGTTGTC | 900 nM |
| REVERSE | (SEQ ID NO: 37) | |

TABLE 4

| | | | Final |
|---|---|---|---|
| | 5′Dye/3′ | | concen- |
| Probe | Quencher | Sequence (5′→3′) | tration |
| CAG | FAM/Iowa | TTACGGTAAACTGCCCACTTG | 250 nM |
| | Black FQ | GCA | |
| | Zen | (SEQ ID NO: 38) | |
| ITR | HEX/Iowa | CACTCCCTCTCTGCGCGCTCG | 250 nM |
| | Black FQ | (SEQ ID NO: 39) | |
| | Zen | | |
| Cap- | FAM/Iowa | AGGGAAGAAACTACATACCTG | 250 nM |
| AAV9 | Black FQ | GACCCA | |
| | Zen | (SEQ ID NO: 40) | |
| Cap- | HEX/Iowa | AAGAAACTGGCTACCTGGACC | 250 nM |
| TS1 | Black FQ | CTG | |
| | Zen | (SEQ ID NO: 41) | |

### In Vitro Transduction Assays

Vector preparations were diluted down to 6.67E9 vg/mL in PBS, in low binding Eppendorf tubes. 15 μL of diluted AAV vector preparations were added in triplicate in 96-well plates to 35 uL cell culture media. 50 μL of HEK293, CHO of hCMEC cell suspensions (4E5, 4E5 and 3E5 cells/mL, respectively) were added to each well. The final MOI for HEK293, CHO and hCMEC cells were 5000, 5000 and 6667, respectively. Plates were incubated at 37C, 5% CO2 for 48 h. Luciferase assays were performed with Britelite plus Reporter Gene Assay System (PerkinElmer, 6066766).

For the library in vitro transduction assay, HEK293T/17 were seeded at a density of 2 million cells per well, in 6-well plates (n=11 replicates). 24 h post seeding, the AAV9/TS purified library was added to each well at a MOI of 10000. 48 h post transduction, mRNA was extracted from transduced cells using the RNAeasy kit RNA extraction kit (Qiagen). 2.5 ug of mRNA was converted to cDNA using Maxima H Minus Reverse Transcriptase (ThermoFisher, EP0751) according to manufacturer instructions and the resulting cDNA was used for NGS library preparation.

### Differential Scanning Fluorimetry (DSF) Assays

The DSF assays were run in line with the method described by Pacouret et al., AAV-ID: A Rapid and Robust Assay for Batch-to-Batch Consistency Evaluation of AAV Preparations, Molecular Therapy, 2017, 25(6) 1375-1386. For each experiment, a 50× working solution of SYPRO Orange was prepared by mixing 495 uL PBS with 5 uL SYPRO Orange 5000×. For each AAV preparation, 25 uL mixes were prepared in a 96-well plate, by mixing 5E10-2.5E12 viral genomes (vg) with 2.5 uL 50×SYPRO Orange (final concentration: 5×) and PBS. The plate was sealed, spun down, and loaded into a Bio-Rad CFX96 qPCR instrument. Samples were incubated for 2 min at 25C, and then subjected to a temperature gradient (25C-99C, 0.8 C/min). SYPRO Orange fluorescence was measured after every temperature increment, using the FRET filter cube of the qPCR instrument. Fluorescence signals F were normalized between 0% and 100% and melting temperatures were defined as the temperature for which the numerical derivative dF/dT reached its maximum.

### Animals

All procedures were performed as approved by the Broad Institute IACUC (0213-06-08). C57BL/6J mice (000664) were purchased from the Jackson Laboratory (JAX). Intravenous administration of rAAV vectors was performed by injecting the virus into the retro-orbital sinus.

### Tissue Processing and Imaging

Tissues were processed as previously described (Huang et al., Delivering genes across the blood-brain barrier: LY6A, a novel cellular receptor for AAV-PHP.B capsids, PLoS ONE, 2019, 14(11) 1-17). Briefly, mice were anesthetized with Euthasol and transcardially perfused with PBS followed by 4% PFA in PBS. Sagittal brain sections were prepared with a vibratome (Leica). Images were taken with a Keyence BZ-X800 fluorescence microscope. All images were taken at the same magnification and exposure.

### Library Viral Genome Extraction

1E11 vg library was incubated with 1000 U/mL Turbonuclease (Sigma T4330-50KU) with 1×DNase I reaction buffer (NEB B0303S) at 37° C. for one hour. The endonuclease solution was inactivated with 0.5M, pH 8.0 EDTA at room temperature for 5 minutes and then at 70° C. for 10 minutes. AAV genomes were released by incubation with 100 g/mL Proteinase K (Qiagen, 19131) in 1M NaCl, 1% N-lauroylsarcosine, and in UltraPure DNase/RNase-Free water at 56° C. for 2 to 16 hours before heat inactivation at 95° C. for 10 minutes. Viral genomes were then purified and concentrated using a Zymo DNA Clean and concentrator (5 ug) kit.

### NGS Sample Preparation

qPCR was performed on extracted AAV genomes, cDNA from transduction assays, and library plasmids to determine the cycle thresholds for each sample type to prevent overamplification. Once cycle thresholds were determined, a first round PCR amplification using equal primer pairs (seq1-seq8 as shown in Table 5, below) (PCR1 Primers) were used to attach Illumina Read 1 and Read 2 sequences using Q5 Hot Start High-Fidelity 2× Master Mix with an annealing temperature of 65° C. for 20 seconds and an extension time of 1 minute. Round 1 PCR products were purified using AMPure XP beads following the manufacturer's protocol and eluted in 25 μL UltraPure Water (ThermoScientific); then, 2 μL was used as input in a second round PCR amplification to attach Illumina adaptors and dual index primers (NEB, E7600S) for five PCR cycles using Q5 HotStart-High-Fidelity 2× Master Mix with an annealing temperature of 65° C. for 20 seconds and an extension time of 1 minute. The second round PCR products were purified using AMPure XP beads following the manufacturer's protocol and eluted in 25 μL UltraPure DNase/RNase-Free distilled water (ThermoScientific). To quantify the amount of second round PCR product for NGS, an Agilent High Sensitivity DNA Kit (Agilent, 5067-4626) was used with an Agilent 2100 Bioanalyzer system. PCR products were then pooled and diluted to 2-4 nM in 10 mM Tris-HCl, pH 8.5 and sequenced on an Illumina NextSeq 550 following the manufacturer's instructions using NextSeq P2 v3 kits (Illumina, 20046813). Reads were allocated as follows: I1: 8, I2: 8, R1: 150, R2: 150.

TABLE 5

| | | |
|---|---|---|
| seq1_ | 5′ | ACGACGCTCTTCCGATCTGACAGAATCAA |
| FOR | handle | caaacgctaaaattcagtgtggc |
| | | (SEQ ID NO: 42) |
| seq2_ | Read 1 | ACGACGCTCTTCCGATCTnGACAGAATCA |
| FOR | | Acaaacgctaaaattcagtgtggc |
| | | (SEQ ID NO: 43) |
| seq3_ | Read 1 | ACGACGCTCTTCCGATCTnnGACAGAATC |
| FOR | | AAcaaacgctaaaattcagtgtggc |
| | | (SEQ ID NO: 44) |
| seq4_ | Read 1 | ACGACGCTCTTCCGATCTnnnGACAGAAT |
| FOR | | CAAcaaacgctaaaattcagtgtggc |
| | | (SEQ ID NO: 45) |
| seq5_ | Read 1 | ACGACGCTCTTCCGATCTnnnnGACAGAA |
| FOR | | TCAAcaaacgctaaaattcagtgtggc |
| | | (SEQ ID NO: 46) |
| seq6_ | Read 1 | ACGACGCTCTTCCGATCTnnnnnGACAGA |
| FOR | | ATCAAcaaacgctaaaattcagtgtggc |
| | | (SEQ ID NO: 47) |
| seq7_ | Read 1 | ACGACGCTCTTCCGATCTnnnnnnGACAG |
| FOR | | AATCAAcaaacgctaaaattcagtgtggc |
| | | (SEQ ID NO: 48) |
| seq8_ | Read 1 | ACGACGCTCTTCCGATCTnnnnnnnGACA |
| FOR | | GAATCAAcaaacgctaaaattcagtgtgg |
| | | c |
| | | (SEQ ID NO: 49) |
| seq1_ | Read 2 | GGAGTTCAGACGTGTGCTCTTCCGATCTC |
| REV | | ATCTCTGTCCTGCCAAACCATACC |
| | | (SEQ ID NO: 50) |
| seq2_ | Read 2 | GGAGTTCAGACGTGTGCTCTTCCGATCTN |
| REV | | CATCTCTGTCCTGCCAAACCATACC |
| | | (SEQ ID NO: 51) |
| seq3_ | Read 2 | GGAGTTCAGACGTGTGCTCTTCCGATCTN |
| REV | | NCATCTCTGTCCTGCCAAACCATACC |
| | | (SEQ ID NO: 52) |
| seq4_ | Read 2 | GGAGTTCAGACGTGTGCTCTTCCGATCTN |
| REV | | NNCATCTCTGTCCTGCCAAACCATACC |
| | | (SEQ ID NO: 53) |
| seq5_ | Read 2 | GGAGTTCAGACGTGTGCTCTTCCGATCTN |
| REV | | NNNCATCTCTGTCCTGCCAAACCATACC |
| | | (SEQ ID NO: 54) |
| seq6_ | Read 2 | GGAGTTCAGACGTGTGCTCTTCCGATCTN |
| REV | | NNNNCATCTCTGTCCTGCCAAACCATACC |
| | | (SEQ ID NO: 55) |
| seq7_ | Read 2 | GGAGTTCAGACGTGTGCTCTTCCGATCTN |
| REV | | NNNNNCATCTCTGTCCTGCCAAACCATAC |
| | | C |
| | | (SEQ ID NO: 56) |
| seq8_ | Read 2 | GGAGTTCAGACGTGTGCTCTTCCGATCTN |
| REV | | NNNNNNCATCTCTGTCCTGCCAAACCATA |
| | | CC |
| | | (SEQ ID NO: 57) |

### NGS Data Processing

Sequencing data was demultiplexed with bcl2fastq (version v2.20.0.422) using the default parameters. The Read 1 sequence (excluding Illumina barcodes) was aligned to a short reference sequence of AAV9:

(SEQ ID NO: 58)
GACAGAATCAACAAACGCTAAAATTCAGTGTGGCCGGACCCAGCAACATG
GCTGTCCAGGGAAGAAACNNNNNNCCTGGACCCNNNTACCGACAACAACG
TGTCTCAACCACTGTGACTCAAAACAACAACAGCGAATTTGCTTGGCCTG
GAGCTTCTTCTTGGGCTCTCAATGGACGTAATAGCTTGATGAATCCTGGA
CCTGCTATGGCCAGCCACAAAGAAGGAGAGGACCGTTTCTTTCCTTTGTC
TGGATCTTTAATTTTTGGCAAACAAGGAACTGGAAGAGACAACGTGGATG
CGGACAAAGTCATGATAACCAACGAAGAAGAAATTAAAACTACTAACCCG
GTAGCAACGGAGTCCTATGGACAAGTGGCCACAAACCACCAGAGTGCCCA
ANNNNNNNNNNNNNNNNNNNNNGCACAGGCGCAGACCGGTTGGGTTCAAA
ACCAAGGAATACTTCCGGGTATGGTTTGGCAGGACAGAGATG

Alignment was performed with bowtie2 (version 2.4.1) (Langmead and Salzberg, Fast gapped-read alignment with Bowtie 2, Nature Methods, 2012, 9(4) 357-359) with the following parameters:
--end-to-end --very-sensitive --np 0 --n-ceil L,21,0.5 --xeq -N 1 --reorder --score-min L,−0.6,−0.6 −5 8 −3 8

The resulting sam files from bowtie2 were sorted by read and compressed to bam files with samtools (version 1.11-2-g26d7c73, htslib version 1.11-9-g2264113) (Danecek et al., Twelve years of SAMtools and BCFtools, GigaScience, 2021, 10(2) 1-4; Li et al., 2009). Python (version 3.8.3) scripts and pysam (version 0.15.4) were used to flexibly extract the 21 nucleotide from the 7mer insertion (read 2) and the 9 nucleotides from residues 478, 479 and 483 diverging between AAV9 and AAVTS1 (read 1). Each read was assigned to one of the following bins: Failed, Invalid, or Valid. Failed reads were defined as reads that did not align to the reference sequence, or that had an in/del in the insertion region (i.e., 29 bases instead of 30 bases). Invalid reads were defined as reads whose 30 bases were successfully extracted but matched any of the following conditions: 1) Any one base of the 30 bases had a quality score (AKA Phred score, QScore) below 20, i.e., error probability >1/100, 2) Any one base was undetermined, i.e., “N”, or 3) The 30 base sequence was not from the synthetic library. Valid reads were defined as reads that did not fit into either the Failed or Invalid bins. The Failed and Invalid reads were collected and analyzed for quality control purposes, and all subsequent analyses were performed on the Valid reads. Count data for valid reads was aggregated per sequence, per sample, and was stored in a pivot table format, with nucleotide sequences on the rows, and samples (Illumina barcodes) on the columns. Sequences not detected in samples were assigned a count of 0.

### Data Normalization

Count data was read-per-million (RPM) normalized to the sequencing depth of each sample j (Illumina barcode) with:

$${r}_{i , j} = \frac{{k}_{i , j}}{{\sum }_{l = 1}^{n} {k}_{l , j}} \times 1000000$$

Where r is the RPM-normalized count, k is the raw count, i=1, 2, . . . , n sequences, and j=1, 2, m samples.

As each biological sample was run in triplicate, we aggregated data for each sample by taking the mean of the RPMs across p replicates of sample s:

$${μ}_{i , s} = \frac{{\sum }_{l = 1}^{p} {r}_{i , l}}{p}$$

Log 2 enrichment for each sequence was defined as:

$${e}_{i , s} = log 2 ( \frac{{μ}_{i , s}}{{μ}_{i , t}} )$$

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered AAV capsid scaffold comprising one or more modified capsid proteins that improve capsid stability to accept further functional modifications, increase viral titer, and/or increase production fitness relative to a reference AAV capsid.

2. The engineered AAV capsid of claim 1, wherein the one or more modifications increase thermal stability, increase resistance to neutralizing antibodies, expand tropism, and or confer greater mechanical stability relative to a reference AAV capsid.

3. The engineered AAV capsid of claim 2, wherein the one or more modifications increase a thermal stability of the AAV capsid by 1, 2, 3, 4, 5, 6, 7, 8, 9, 10, 11, 12, 13, 14, 15, 16, 17, 18, 19, or 20° C. relative to an AAV capsid that does not comprise the one or more modified proteins.

4. The engineered AAV capsid of anyone of the preceding claims, wherein the AAV capsid is a AAV9 capsid and the one or more modifications are selected from the group consisting of N41D, G56F, V211M, Q233T, D349E, E361Q, D384N, E416T, N419D, K449R, Q456T, V465Q, Y478W, I479L, S483C, P504T, S507T, S508K, W509Y, A510H, E529D, G530D, H584L, Q597N, M640L, D665A, N668A, and E712D.

5. The engineered AAV capsid of claim 4, wherein at least one modification is selected from the group consisting of D384N, S483C, P504T, S508K, and W509Y, and at least one modification is selected from the group consisting of E529D, G530D, Y478W, and I479L.

6. The engineered AAV capsid of claim 4, wherein the one or more modifications comprise D384N, S483C, P504T, S508K, and W509Y.

7. The engineered AAV capsid of claim 4, wherein the one or more modifications comprise of D384N, S483C, P504T, S508K, W509Y, E529D, and G530D.

8. The engineered AAV capsid of claim 4, wherein the one or more modifications comprise D384N, E529D, and G530D.

9. The engineered AAV capsid of claim 4, wherein the one or more modifications comprise N41D, G56F, V211M, Q233T, D384N, Y478W, I479L, S483C, P504T, S508K, W509Y, E529D, G530D, and M640L.

10. The engineered AAV capsid of claim 4, wherein the one or more modifications comprise D384N, S483C, P504T, S508K, W509Y, E529D, and G530D.

11. The engineered AAV capsid of claim 4, wherein the one or more modifications comprise D384N, S483C, P504T, S508K, W509Y, E529D, G530D, and Q597N.

12. The engineered AAV capsid of claim 4, wherein the one or more modifications comprise S483C, P504T, S508K, W509Y, E529D, G530D, and Q597N.

13. The engineered AAV capsid of claim 4, wherein the one or more modifications comprise D384N, S483C, P504T, S508K, W509Y, and Q597N.

14. The engineered AAV capsid of claim 4, wherein the one or more modifications comprise S483C, P504T, S508K, W509Y, and Q597N.

15. The engineered AAV capsid of claim 4, wherein the one or more modifications comprise D384N, S483C, S508K, W509Y, E529D, G530D, and Q597N.

16. The engineered AAV capsid of claim 4, wherein the one or more modifications comprise D384N, S483C, P504T, E529D, G530D, and Q597N.

17. The engineered AAV capsid of claim 4, wherein the one or more modifications comprise D384N, S483C, P504T, W509Y, E529D, G530D, and Q597N.

18. The engineered AAV capsid of claim 4, wherein the one or more modifications comprise Q597N.

19. The engineered AAV capsid of claim 4, wherein the one or more modifications comprise D384N.

20. The engineered AAV capsid of claim 4, wherein the one or more modifications comprise S483C.

21. The engineered AAV capsid of claim 4, wherein the one or more modifications comprise P504T.

22. The engineered AAV capsid of claim 4, wherein the one or more modifications comprise S507T and S508K.

23. The engineered AAV capsid of claim 4, wherein the one or more modifications comprise S507T, S508K, W509Y, and A510H.

24. The engineered AAV capsid of claim 4, wherein the one or more modifications comprise S508K and W509Y.

25. The engineered AAV capsid of anyone of claims 1 to 4, wherein the AAV capsid is a AAV1 capsid and the one or more modifications are selected from the group consisting of E418D, R465Q, S467G, S507T, N510H, I517L, M541L, A568T, F577Y, F584L, H597N, M599Q, N642H, T665A, V699I, and P735N.

26. The engineered AAV capsid of anyone of claims 1 to 4, wherein the AAV capsid is a AAV2 capsid and the one or more modifications are selected from the group consisting of S207G, M235L, V372I, N449Q, A467P, I470M, E499N, Y500F, S547A, A590P, V600A, S658P, T713A.

27. The engineered AAV capsid of any one of claims 1 to 4, wherein the AAV capsid is a AAV3B capsid and the one or more modifications are selected from the group consisting of; S205A, Q233T, K310R, V372I, L489V, A505G, S507T, H538S, N540V, E546Q, T548A, T549G, M648L, and T714A.

28. The engineered AAV capsid of any one of claims 1 to 4, wherein the AAV capsid is a AAV6 capsid and the one or more modifications are selected from the group consisting of; R465Q, S467G, S507T, N510H, I517L, K531E, M541L, A568T, F577Y, H597N, M599Q, T665A, V699I, P735N.

29. The engineered AAV capsid of any one of claims 1 to 4, wherein the AAV capsid is a AAV7 capsid and the one or more modifications are selected from the group consisting of; V204M, T265S, S388A, S416T, Y466S, G468A, F486Y, K553N, L560M, P569T, F706Y, Q709S, and G711N.

30. The engineered AAV capsid of any one of claims 1 to 4, wherein the AAV capsid is a AAV8 capsid and the one or more modifications are selected from the group consisting of; S224N, T415S, G468A, A507G, G508A, A520V, N540S, I542V, N549G, A551G, A555V, S667A, N670A, S712N.

31. The engineered AAV capsid of any one of claims 1 to 4, wherein the AAV capsid is a AAVrh10 capsid and the one or more modifications are selected from the group consisting of, S224N, N263S, E330D, K333T, I343V, Q417T, T493K, S559N, A591T, V595T, D659N, L669F, and T722V.

32. The engineered AAV capsid of anyone of claims 1 to 4, wherein the AAV capsid is a AAVrh8 capsid and the one or more modifications are selected from the group consisting of; L389V, V479L, A507T, F509Y, K510H, D531E, G552N, L559M, A568T, N584L, H597N, I602L, and N668A

33. The engineered AAV capsid of any one of the preceding claims further comprising at least one n-mer insertion that re-targets or improves transducibility of the engineered AAV capsid.

34. The engineered AAV capsid of claim 33, wherein the n-mer consists of a 7-mer.

35. The engineered AAV capsid of claim 34, wherein the n-mer is inserted at amino acid 588 of AAV9 VP1 or an analogous position of AAV1, AAV2, AAV3B, AAV6, AAV7, AAV8, AAVrh10, AAVrh8, or AAV-PHP.eB.

36. A vector system comprising one or more vectors encoding the engineered AAV capsid of any one of the preceding claims.

37. A composition comprising the engineered AAV capsid of any one of claims 1 to 35.

38. A method of delivering cargos to cells comprising;
administering an engineered AVV particle comprising the engineered capsid of any one of claims 1 to 35 to a population of cells, the engineered AAV particle further comprising a cargo.

39. The method of claim 38, wherein the cargo is a recombinant genome polynucleotide comprising a transgene operably linked to a regulatory sequence and flanked by AAV ITR sequences
